(19) United States
(12) Patent Application Publication
(10) Pub. No.: US 2024/0158348 A1
(43) Pub. Date: May 16, 2024

(54) HERBICIDAL CYCLIC AMIDES N-SUBSTITUTED WITH A HALOALKYLSULFONYLANILIDE GROUP

(71) Applicant: FMC Corporation, Philadelphia, PA (US)

(72) Inventors: Thomas Paul SELBY, Hockessin, DE (US); Wandi ZHANG, Shanghai (CN); Alison Mary LEVENS, Wilmington, DE (US)

(21) Appl. No.: 18/277,104

(22) PCT Filed: Feb. 15, 2022
(86) PCT No.: PCT/US2022/016430

§ 371 Date: Aug. 14, 2023

Related U.S. Application Data
(60) Provisional application No. 63/149,711, filed on Feb. 16, 2021.

(51) Int. Cl. C07D 207/12; A01N 25/32; A01N 47/02; A01P 13/00; C07D 211/40

(52) U.S. Cl. CPC C07D 207/12; A01N 25/32; A01N 47/02; A01P 13/00; C07D 211/40

(57) ABSTRACT

Disclosed are compounds of Formula 1, all stereoisomers, N-oxides, and salts thereof,

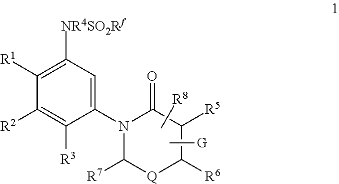

wherein R¹ through R⁸, R^{f}, Q and G are as defined in the Disclosure.

Also disclosed are compositions containing the compounds of Formula 1 and methods for controlling undesired vegetation comprising contacting the undesired vegetation or its environment with an effective amount of a compound or a composition of the invention.

### FIELD OF THE DISCLOSURE

This invention relates to certain haloalkyl sulfonanilides, their N-oxides, salts and compositions, and methods of their use for controlling undesirable vegetation.

### BACKGROUND OF THE DISCLOSURE

The control of undesired vegetation is extremely important in achieving high crop efficiency. Achievement of selective control of the growth of weeds especially in such useful crops as rice, soybean, sugar beet, maize, potato, wheat, barley, tomato and plantation crops, among others, is very desirable. Unchecked weed growth in such useful crops can cause significant reduction in productivity and thereby result in increased costs to the consumer. The control of undesired vegetation in noncrop areas is also important. Many products are commercially available for these purposes, but the need continues for new compounds that are more effective, less costly, less toxic, environmentally safer or have different sites of action.

### SUMMARY OF THE DISCLOSURE

This invention is directed to compounds of Formula 1, all stereoisomers, N-oxides, and salts thereof, agricultural compositions containing them and their use as herbicides:

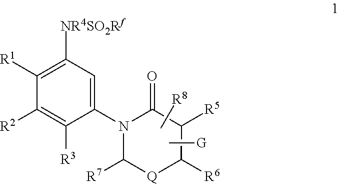

wherein
R¹ is H, C₁-C₇ alkyl, halogen, CN, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₃-C₇ haloalkynyl, C₂-C₇ alkoxyalkyl, C₁-C₇ alkoxy, C₁-C₅ alkylthio, C₂-C₃ alkoxycarbonyl or C₂-C₇ haloalkoxyalkyl;
R² is H, C₁-C₇ alkyl, halogen, CN, C₁-C₇ haloalkyl, C₁-C₇ alkoxy or C₁-C₅ alkylthio;
R³ is H, C₁-C₇ alkyl, halogen, CN, C₂-C₆ alkenyl, C₂-C₇ alkynyl, C₃-C₇ cycloalkyl, C₂-C₄ cyanoalkyl, C₁-C₇ haloalkyl, C₂-C₇ haloalkenyl, C₃-C₇ haloalkynyl, C₂-C₇ alkoxyalkyl, C₁-C₇ alkoxy, C₁-C₅ alkylthio, C₂-C₃ alkoxycarbonyl or C₂-C₇ haloalkoxyalkyl;
R⁴ is H, C(═O)R¹⁴, —C(═S)R¹⁴, —CO₂R¹⁴, —C(═O)SR¹⁴, —S(O)₂R¹⁴, C(═O)NR¹³R¹⁴, —S(O)₂NR¹³R¹⁴, CH₂OC(═O)OR¹⁴, CH₂OC(═O)NR¹³R¹⁴ or CH₂OC(═O)R¹⁴; or propargyl, allyl or benzyl.
R⁵ is H, C₂-C₆ alkenyl, C₂-C₇ haloalkenyl, C₂-C₆ alkynyl, C₃-C₇ cycloalkyl, C₄-C₇ cycloalkylalkyl, C₂-C₄ cyanoalkyl, C₁-C₇ haloalkyl, C₃-C₇ alkylthioalkyl, C₁-C₇ haloalkoxy, C₂-C₇ alkoxyalkyl or C₄-C₇ alkylcycloalkyl;
R⁶ is H, C₁-C₇ alkyl, halogen, CN, C₁-C₅ alkylthio, C₂-C₃ alkoxycarbonyl, C₂-C₆ alkenyl, C₂-C₆ alkynyl, C₃-C₇ cycloalkyl, C₄-C₇ cycloalkylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ haloalkoxyalkyl or C₄-C₇ alkylcycloalkyl;
R⁷ is H, C₁-C₇ alkyl, halogen, CN, C₁-C₅ alkylthio, C₂-C₃ alkoxycarbonyl, C₂-C₆ alkenyl, C₂-C₆ alkynyl, C₃-C₇ cycloalkyl, C₄-C₇ cycloalkylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ haloalkoxyalkyl or C₄-C₇ alkylcycloalkyl;
R⁸ is H, C₁-C₇ alkyl, C₂-C₆ alkenyl, C₂-C₆ alkynyl, C₃-C₇ cycloalkyl, C₄-C₇ cycloalkylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ alkoxyalkyl or C₄-C₇ alkylcycloalkyl;
Q is CHR⁹, O or a direct bond;
R⁹ is H, C₁-C₇ alkyl, halogen, CN, C₁-C₅ alkylthio, C₂-C₃ alkoxycarbonyl, C₂-C₆ alkenyl, C₂-C₆ alkynyl, C₃-C₇ cycloalkyl, C₄-C₇ cycloalkylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ alkoxyalkyl, C₂-C₇ haloalkoxyalkyl or C₄-C₇ alkylcycloalkyl;
G is OR¹⁰, SR¹⁰, SOR¹⁰ or SO₂R¹⁰; or
G and R⁵ are taken together to form N—OR¹⁵; R¹⁰ is H, C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₁-C₆ nitroalkyl, C₃-C₆ alkylcarboalkyl, C₃-C₆ alkoxycarboalkyl, C₂-C₇ haloalkoxyalkyl, benzyl or C₃-C₆ alkylcarboalkoxy; or
R¹⁰ is selected from the group consisting of

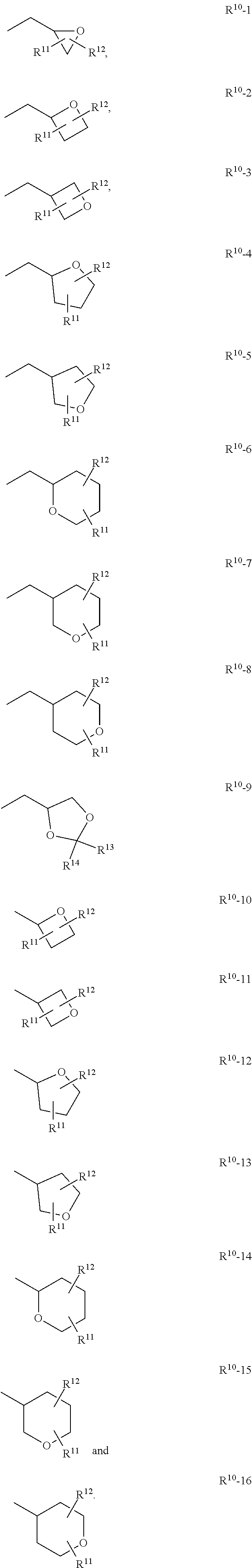

R¹¹ is H, C₁-C₇ alkyl, C₂-C₆ alkenyl, C₂-C₆ alkynyl, C₃-C₇ cycloalkyl, C₄-C₇ cycloalkylalkyl, C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ alkoxyalkyl or C₄-C₇ alkylcycloalkyl;
R¹² is H, C₁-C₇ alkyl, C₂-C₆ alkenyl, C₂-C₆ alkynyl, C₃-C₇ cycloalkyl, C₄-C₇ cycloalkylalkyl or C₇ haloalkyl;
each R¹³ and R¹⁴ is independently H, C₁-C₇ alkyl, C₂-C₆ alkenyl, C₂-C₆ alkynyl, C₃-C₇ cycloalkyl, C₄-C₇ cycloalkylalkyl, C₂-C₃ cyanoalkyl, C₁-C₇ haloalkyl, C₂-C₇ haloalkenyl, C₂-C₇ alkylalkoxyalkyl, C₃-C₇ alkylthioalkyl, C₁-C₇ alkoxy; C₂-C₇ alkoxyalkyl, C₄-C₇ alkylcycloalkyl, Ph or benzyl;
R^{f} is C₁-C₇ haloalkyl;
G and R⁸ can be attached to any ring carbon(s) with available valency, said ring is the cyclic amide ring shown in Formula 1;
each R¹¹ or R¹² can be attached to any ring carbon(s) with available valency, said ring is illustrated in R¹⁰-1 through R¹⁰-16 as above; and
R¹⁵ is H, C₁-C₆ alkyl, C₁-C₆ haloalkyl, C₂-C₆ alkenyl, C₂-C₆ alkynyl or C₄-C₇ cycloalkylalkyl.

More particularly, this invention pertains to a compound of Formula 1, all stereoisomers, an N-oxide or a salt thereof. This invention also relates to a herbicidal composition comprising a compound of the disclosure (i.e. in a herbicidally effective amount) and at least one component selected from the group consisting of surfactants, solid diluents and liquid diluents. This invention further relates to a method for controlling the growth of undesired vegetation comprising contacting the vegetation or its environment with a herbicidally effective amount of a compound of the disclosure (e.g., as a composition described herein).

This invention also includes a herbicidal mixture comprising (a) a compound selected from Formula 1, all stereoisomers, N-oxides, and salts thereof, and (b) at least one additional active ingredient selected from (b1) through (b16), and salts of compounds of (b1) through (b16), as described below.

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process, method, article or apparatus that comprises a list of elements is not necessarily limited to only those elements but may include other elements not expressly listed or inherent to such composition, mixture, process, method, article or apparatus.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, method or apparatus that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to herein, the term “seedling”, used either alone or in a combination of words means a young plant developing from the embryo of a seed.

As referred to herein, the term “broadleaf” used either alone or in words such as “broadleaf weed” means dicot or dicotyledon, a term used to describe a group of angiosperms characterized by embryos having two cotyledons.

In the above recitations, the term “alkyl”, used either alone or in compound words such as “alkylthio” or “haloalkyl” includes straight-chain or branched alkyl, such as, methyl, ethyl, n-propyl, i-propyl or the different butyl, pentyl or hexyl isomers. “Alkenyl” includes straight-chain or branched alkenes such as ethenyl, 1-propenyl, 2-propenyl, 1-butenyl, 2-butenyl and the different butenyl, pentenyl and hexenyl isomers. “Alkenyl” also includes polyenes such as 1,2-propadienyl and 2,4-hexadienyl. “Alkenylalkyl” denotes alkenyl substitution on alkyl. Examples of “alkenylalkyl” include CH₂═CHCH₂, CH₃CH═CHCH₂, CH₂═CHCH₂CH₂, CH₂═CHCH(CH₃)CH₂ and the different alkenylalkyl isomers. “Alkenylalkyl” is a subset of “alkenyl”. “Alkynyl” includes straight-chain or branched alkynes such as ethynyl, 1-propynyl, 2-propynyl, CH═CCH₂CH₂, CH₃C==CCH₂ and the different butynyl, pentynyl and hexynyl isomers. “Alkynyl” can also include moieties comprised of multiple triple bonds such as 2,5-hexadiynyl. “Alkynylalkyl” denotes alkynyl substitution on alkyl. Examples of “alkynylalkyl” include CH═CCH₂, CH₃C==CCH₂, CH═CCH₂CH₂, CH═CCH(CH₃)CH₂ and the different alkynylalkyl isomers. “Alkynylalkyl” is a subset of “alkynyl”. “Alkylene” denotes a straight-chain or branched alkanediyl. Examples of “alkylene” include CH₂, CH₂CH₂, CH(CH₃), CH₂CH₂CH₂, CH₂CH(CH₃) and the different butylene isomers. “Alkenylene” denotes a straight-chain or branched alkenediyl containing one olefinic bond. Examples of “alkenylene” include CH═CH, CH₂CH═CH, CH═C(CH₃) and the different butenylene isomers. “Alkynylene” denotes a straight-chain or branched alkynediyl containing one triple bond. Examples of “alkynylene” include C≡C, CH₂C≡C, C≡CCH₂ and the different butynylene isomers.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkoxyalkoxy” denotes alkoxy substitution on alkoxy. “Alkenyloxy” includes straight-chain or branched alkenyloxy moieties. Examples of “alkenyloxy” include H₂C═CHCH₂O, (CH₃)₂C═CHCH₂O, (CH₃)CH═CHCH₂O, (CH₃)CH═C(CH₃)CH₂O and CH₂═CHCH₂CH₂O. “Alkynyloxy” includes straight-chain or branched alkynyloxy moieties. Examples of “alkynyloxy” include HC═CCH₂O, CH₃C═CCH₂O and CH₃C═CCH₂CH₂O. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂. “Alkylthioalkoxy” denotes alkylthio substitution on alkoxy. “Alkyldithio” denotes branched or straight-chain alkyldithio moieties. Examples of “alkyldithio” include CH₃SS—, CH₃CH₂SS—, CH₃CH₂CH₂SS—, (CH₃)₂CHSS— and the different butyldithio and pentyldithio isomers. “Cyanoalkyl” denotes an alkyl group substituted with one cyano group. Examples of “cyanoalkyl” include NCCH₂, NCCH₂CH₂ and CH₃CH(CN)CH₂. “Alkylamino”, “dialkylamino”, “alkenylthio”, “alkenylsulfinyl”, “alkenylsulfonyl”, “alkynylthio”, “alkynylsulfinyl”, “alkynylsulfonyl”, and the like, are defined analogously to the above examples.

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. Examples of “alkylcycloalkylalkyl” include 2-methylcyclopropylmethyl, methylcyclopentylethyl, and other alkylcycloalkyl moieties bonded to straight-chain or branched alkyl groups. The term “cycloalkoxy” denotes cycloalkyl linked through an oxygen atom such as cyclopentyloxy and cyclohexyloxy. “Cycloalkylalkoxy” denotes cycloalkylalkyl linked through an oxygen atom attached to the alkyl chain. Examples of “cycloalkylalkoxy” include cyclopropylmethoxy, cyclopentylethoxy, and other cycloalkyl moieties bonded to straight-chain or branched alkoxy groups. “Cyanocycloalkyl” denotes a cycloalkyl group substituted with one cyano group. Examples of “cyanocycloalkyl” include 4-cyanocyclohexyl and 3-cyanocyclopentyl. “Cycloalkenyl” includes groups such as cyclopentenyl and cyclohexenyl as well as groups with more than one double bond such as 1,3- and 1,4-cyclohexadienyl.

The term “halogen”, either alone or in compound words such as “haloalkyl” or when used in descriptions such as “alkyl substituted with halogen” includes fluorine, chlorine, bromine or iodine. Further, when used in compound words such as “haloalkyl” or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “halocycloalkyl”, “haloalkoxy”, “haloalkylthio”, “haloalkenyl”, “haloalkynyl”, and the like, are defined analogously to the term “haloalkyl”. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—. Examples of “haloalkenyl” include (C₁)₂C═CHCH₂— and CF₃CH₂CH═CHCH₂—. Examples of “haloalkynyl” include HC═CCHCl—, CF₃C═C—, CCl₃C═C— and FCH₂C═CCH₂—. Examples of “haloalkoxyalkoxy” include CF₃OCH₂O—, C₁CH₂CH₂OCH₂CH₂O—, Cl₃CCH₂OCH₂O— as well as branched alkyl derivatives. Examples of “haloalkoxyalkyl” include CF₃OCH₂—, ClCH₂CH₂OCH₂CH₂, Cl₃CCH₂OCH₂CH₂— as well as branched alkyl derivatives.

“Alkylcarbonyl” denotes a straight-chain or branched alkyl moieties bonded to a C(═O) moiety. Examples of “alkylcarbonyl” include CH₃C(═O)—, CH₃CH₂CH₂C(═O)— and (CH₃)₂CHC(═O)—. “Alkylcarboalkoxy” denotes a straight-chain or branched alkoxy substituted with alkylcarbonyl group. Examples of “Alkylcarboalkoxy” include CH₃C(═O) CH₂O—, CH₃CH₂CH₂C(═O)CH₂O— and (CH₃)₂CHC(═O)CH₂CH₂O—. Examples of “alkoxycarbonyl” include CH₃OC(═O)—, CH₃CH₂OC(═O)—, CH₃CH₂CH₂OC(═O)—, (CH₃)₂CHOC(═O)— and the different butoxy- or pentoxycarbonyl isomers.

“Alkoxycarboalkyl” denotes a straight-chain or branched alkyl substituted with alkoxycarbonyl group. Examples of “alkoxycarboalkyl” include CH₃OC(═O)CH₂—, CH₃CH₂OC(═O)CH₂CH₂—, CH₃CH₂CH₂OC(═O)CH₂—, (CH₃)₂CHOC(═O)CH(CH₃)CH₂— and the different butoxy- or pentoxycarbonylalkyl isomers.

The total number of carbon atoms in a substituent group is indicated by the “Cᵢ-Cⱼ” prefix where i and j are numbers from 1 to 7. In other words, i and j indicate the total number of carbon atoms in this group, and i through j indicates the range of the possible total number of the carbon atoms in the group. For example, C₁-C₄ alkylsulfonyl designates methylsulfonyl through butylsulfonyl; C₂-C₆ alkenyl designates ethenyl through hexenyl, and the different propenyl, butenyl, pentenyl and hexenyl isomers. C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

When a group contains a substituent which can be hydrogen, for example R², then when this substituent is taken as hydrogen, it is recognized that this is equivalent to said group being unsubstituted at this position. When one or more positions on a group are said to be “not substituted” or “unsubstituted”, then hydrogen atoms are attached to take up any free valency.

For substituents G, R⁸, R¹¹ or R¹², the attachment point of these substituents is illustrated as floating, which means each of these substituents can be attached to any of the available carbons on the ring, to which they are attached, by replacement of a hydrogen atom. For example, G or R⁸ can be attached to any ring carbon(s) with available valency by replacement of a hydrogen atom, said ring is the cyclic amide ring as shown in Formula 1. For example, when Q is CHR⁹, G can be attached to the said carbon by replacement of the H of CHR⁹ to form a moiety of C(G)R⁹. R¹¹ or R¹² can be attached to any ring carbon(s) with available valency by replacement of a hydrogen atom, said ring is illustrated in R¹⁰-1 through R¹⁰-16 in the Summary of The Disclosure. In this disclosure, the cyclic amide ring always has the substituent G.

Unless otherwise indicated, a “ring” as a component of Formula 1 is carbocyclic or heterocyclic. For example, a cyclic amide ring is a ring containing a N—CO group, it can optionally contain more heteroatom(s) as the ring member(s). The term “ring member” refers to an atom or other moiety (e.g., C(═O), C(═S), S(O) or S(O)₂) forming the backbone of a ring or ring system.

Some non-limiting examples of cyclic amide rings in this disclosure are illustrated in Exhibit 1 wherein each structure is associated with a L-# and the # is a number. When the substituent on the cyclic amide ring is G, but not specified for other substituents on the same carbon to which G is bonded (e.g., L-2, L-4, L-6, L-8, L-10, L-12, L-14, L-16 and L-18) then H or R⁸ can take up the remaining valance on said carbon. G and R⁵ can also be taken together to form N—OR¹⁵, wherein the N is attached to the carbon ring member through a double bond to form an oxime moiety, such as in L-19.

### Exhibit 1

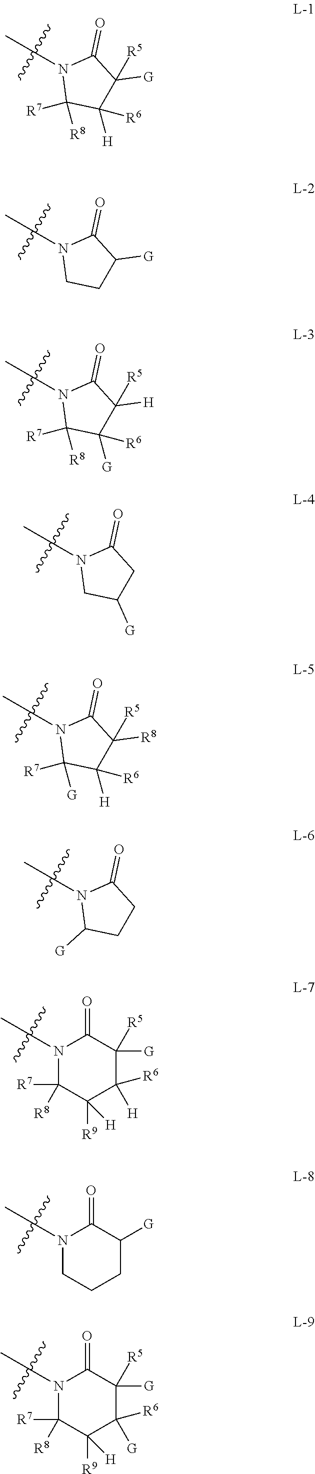

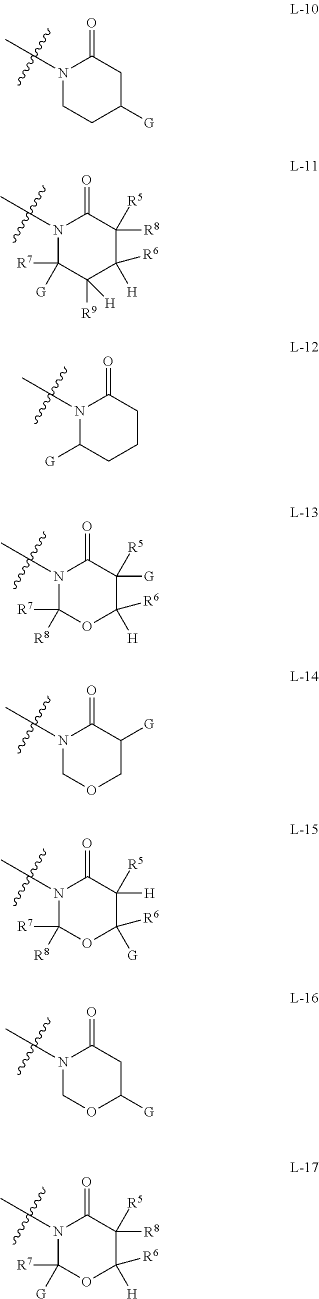

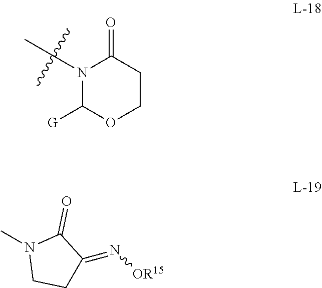

In one specific embodiment, G and R⁵ can be taken together to form N—OR¹⁵, wherein the N is attached to the carbon ring member through a double bond to form an oxime moirty, as shown below.

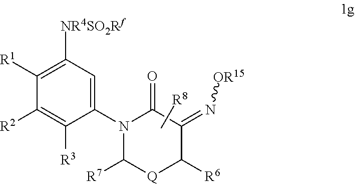

The terms “heterocyclic ring”, “heterocycle” or “heterocyclic ring system” denote a ring or ring system in which at least one atom forming the ring backbone is not carbon, e.g., nitrogen, oxygen or sulfur. Typically, a heterocyclic ring contains no more than 4 nitrogens, no more than 2 oxygens and no more than 2 sulfurs. Unless otherwise indicated, a heterocyclic ring can be a saturated, partially unsaturated or fully unsaturated ring. When a fully unsaturated heterocyclic ring satisfies Hückel's rule, then said ring is also called a “heteroaromatic ring” or “aromatic heterocyclic ring”. Unless otherwise indicated, heterocyclic rings and ring systems can be attached through any available carbon or nitrogen by replacement of a hydrogen on said carbon or nitrogen.

“Aromatic” indicates that each of the ring atoms is essentially in the same plane and has a p-orbital perpendicular to the ring plane, and that (4n+2) π electrons, where n is a positive integer, are associated with the ring to comply with Hückel's rule. The term “aromatic ring system” denotes a carbocyclic or heterocyclic ring system in which at least one ring of the ring system is aromatic. The term “aromatic carbocyclic ring system” denotes a carbocyclic ring system in which at least one ring of the ring system is aromatic. The term “aromatic heterocyclic ring system” denotes a heterocyclic ring system in which at least one ring of the ring system is aromatic. The term “nonaromatic ring system” denotes a carbocyclic or heterocyclic ring system that may be fully saturated, as well as partially or fully unsaturated, provided that none of the rings in the ring system are aromatic. The term “nonaromatic carbocyclic ring system” in which no ring in the ring system is aromatic. The term “nonaromatic heterocyclic ring system” denotes a heterocyclic ring system in which no ring in the ring system is aromatic.

The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

In Formula 1, when G is OR¹⁰, SR¹⁰, SOR¹⁰ or SO₂R¹⁰, R¹⁰ can be (among others) J. Some non-limiting examples of J are illustrated in the table of Exhibit 2 wherein each structure is associated with a J-# and the # is a number.

### Exhibit 2

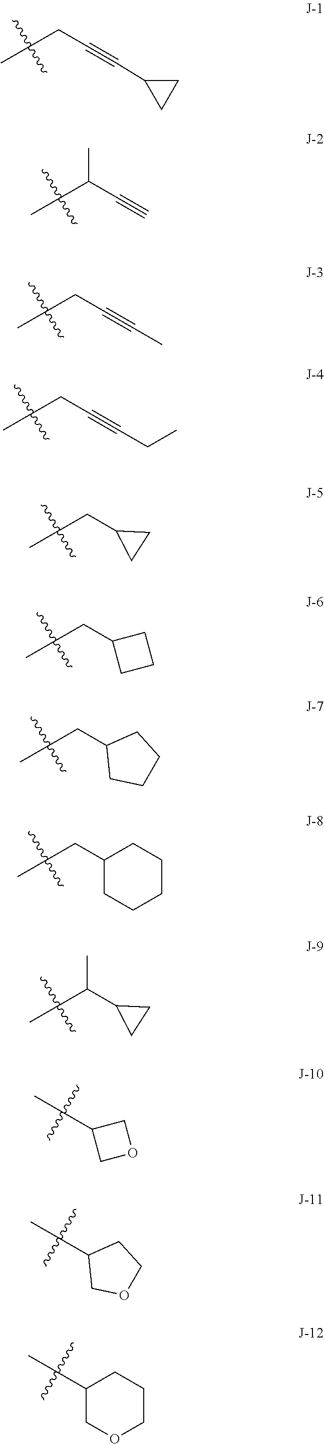

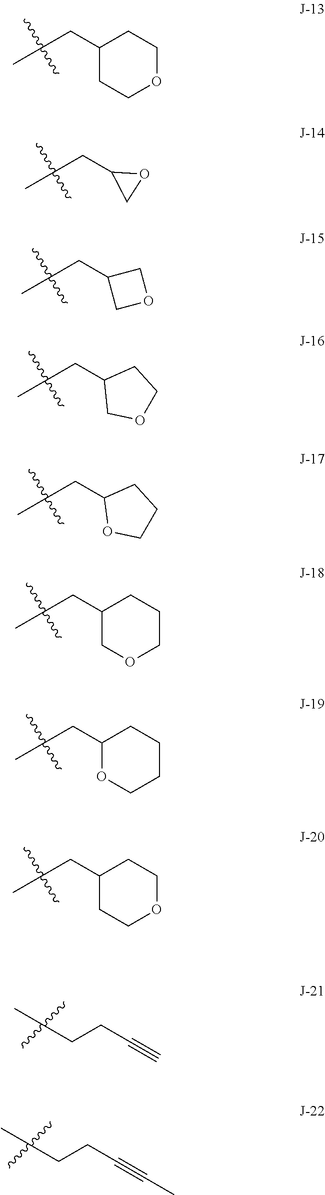

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

For example, when G and R⁵ are different and attached to the same carbon, the compound of Formula 1 may have at least two stereoisomers. The two stereoisomers are depicted as Formula 1′ and Formula 1″ with the chiral center identified with an asterisk (*). For a comprehensive discussion of all aspects of stereoisomerism, see Ernest L. Eliel and Samuel H. Wilen, Stereochemistry of Organic Compounds, John Wiley & Sons, 1994.

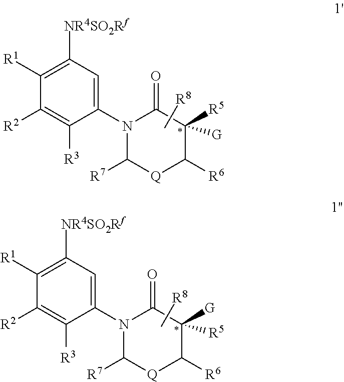

Molecular depictions drawn herein follow standard conventions for depicting stereochemistry. To indicate stereoconfiguration, bonds rising from the plane of the drawing and towards the viewer are denoted by solid wedges wherein the broad end of the wedge is attached to the atom rising from the plane of the drawing towards the viewer. Bonds going below the plane of the drawing and away from the viewer are denoted by dashed wedges wherein the broad end of the wedge is attached to the atom further away from the viewer. Constant width lines indicate bonds with a direction opposite or neutral relative to bonds shown with solid or dashed wedges; constant width lines also depict bonds in molecules or parts of molecules in which no particular stereoconfiguration is intended to be specified.

This invention comprises racemic mixtures, for example, equal amounts of the enantiomers of Formulae 1′ and 1″. In addition, this invention includes compounds that are enriched compared to the racemic mixture in an enantiomer of Formula 1. Also included are the essentially pure enantiomers of compounds of Formula 1, for example, Formula 1′ or Formula 1″.

When enantiomerically enriched, one enantiomer is present in greater amounts than the other, and the extent of enrichment can be defined by an expression of enantiomeric excess (“ee”), which is defined as (2x−1) 100%, where x is the mole fraction of the dominant enantiomer in the mixture (e.g., an ee of 20% corresponds to a 60:40 ratio of enantiomers).

Preferably the compositions of this invention have at least a 50% enantiomeric excess; more preferably at least a 75% enantiomeric excess; still more preferably at least a 90% enantiomeric excess; and the most preferably at least a 94% enantiomeric excess of the more active isomer. Of particular note are enantiomerically pure embodiments of the more active isomer.

Compounds of Formula 1 may comprise additional chiral centers. For example, substituents and other molecular constituents, such as G and R⁵, may themselves contain chiral centers. This invention comprises racemic mixtures as well as enriched and essentially pure stereoconfigurations at these additional chiral centers.

Compounds of this invention can exist as one or more conformational isomers due to any restricted bond rotation in Formula 1. This invention comprises mixtures of conformational isomers. In addition, this invention includes compounds that are enriched in one conformer relative to others.

Compounds of Formula 1 typically exist in more than one form, and Formula 1 thus include all crystalline and non-crystalline forms of the compounds they represent. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound of Formula 1 can exhibit beneficial effects (e.g., suitability for preparation of useful formulations, improved biological performance) relative to another polymorph or a mixture of polymorphs of the same compound of Formula 1. Preparation and isolation of a particular polymorph of a compound of Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation ofN-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus, a wide variety of salts of a compound of Formula 1 are useful for control of undesired vegetation (i.e. are agriculturally suitable). The salts of a compound of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid or phenol, salts also include those formed with organic or inorganic bases such as pyridine, triethylamine or ammonia or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Embodiments of the present invention as described in the Summary of the Disclosure include those wherein a compound of Formula 1 is as described in any of the following Embodiments:
Embodiment 1. A compound of Formula 1, as described in the Summary of the Disclosure, all stereoisomers, N-oxides, and salts thereof, agricultural compositions containing them and their use as herbicides as described in the Summary of the Disclosure.
Embodiment 2. A compound of Formula 1 or Embodiment 1 wherein Q is CHR⁹, O or a direct bond.
Embodiment 2a. A compound of Formula 1 or Embodiment 2 wherein Q is CHR⁹ or a direct bond.
Embodiment 2b. A compound of Formula 1 or Embodiment 2a wherein Q is CHR⁹.
Embodiment 2c. A compound of Formula 1 or Embodiment 2a wherein Q is direct bond.
Embodiment 2d. A compound of Formula 1 or Embodiment 2 wherein Q is O.
Embodiment 3. A compound of Formula 1 or any one of the preceding Embodiments wherein R¹ is H, C₁-C₇ alkyl, halogen, C₂-C₆ alkenyl, C₂-C₆ alkynyl, C₃-C₇ cycloalkyl, C₁-C₇ haloalkyl.
Embodiment 3a. A compound of Embodiment 3 wherein R¹ is H, C₁-C₇ alkyl, halogen, C₃-C₇ cycloalkyl.
Embodiment 3b. A compound of Embodiment 3a wherein R¹ is H, C₁-C₃ alkyl, halogen or C₃-C₄ cycloalkyl.
Embodiment 3c. A compound of Embodiment 3b wherein R¹ is H, Me, halogen or cyclopropyl.
Embodiment 3d. A compound of Embodiment 3c wherein R¹ is H, Me, F, Cl, Br or cyclopropyl.
Embodiment 3e. A compound of Embodiment 3d wherein R¹ is Me or C₁.
Embodiment 3f A compound of Embodiment 3e wherein R¹ is Me.
Embodiment 3g. A compound of Embodiment 3e wherein R¹ is C₁.
Embodiment 3h. A compound of Embodiment 3d wherein R¹ is H.
Embodiment 4. A compound of Formula 1 or any one of the preceding Embodiments wherein R² is H, C₁-C₇ alkyl, halogen, CN, C₁-C₇ haloalkyl, C₁-C₇ alkoxy or C₁-C₅ alkylthio.
Embodiment 4a. A compound of Embodiment 4 wherein R² is H, C₁-C₇ alkyl, halogen or CN.
Embodiment 4b. A compound of Embodiment 4a wherein R² is H, Me, F, Cl or CN.
Embodiment 4c. A compound of Embodiment 4b wherein R² is H or F.
Embodiment 4d. A compound of Embodiment 4c wherein R² is H.
Embodiment 4e. A compound of Embodiment 4c wherein R² is F.
Embodiment 5. A compound of Formula 1 or any one of the preceding Embodiments wherein R³ is H, C₁-C₇ alkyl, halogen, CN, C₂-C₆ alkenyl, C₃-C₇ alkynyl, C₃-C₇ cycloalkyl, C₂-C₄ cyanoalkyl, C₁-C₇ haloalkyl, C₂-C₇ haloalkenyl, C₃-C₇ haloalkynyl, C₂-C₇ alkoxyalkyl, C₁-C₇ alkoxy, C₁-C₅ alkylthio, C₂-C₃ alkoxycarbonyl or C₂-C₇ haloalkoxyalkyl.
Embodiment 5a. A compound of Embodiment 5 wherein R³ is H, C₁-C₇ alkyl, halogen, CN, C₁-C₇ alkoxy or C₁-C₇ haloalkyl.
Embodiment 5b. A compound of Embodiment 5a wherein R³ is H, Me, F, Cl, CN, OMe or CF₃.
Embodiment 5c. A compound of Embodiment 5b wherein R³ is Me or F.
Embodiment 5d. A compound of Embodiment 5c wherein R³ is Me.
Embodiment 6. A compound of Formula 1 or any one of the preceding Embodiments wherein R⁴ is H, C(═O)R¹⁴, C(═S)R¹⁴, C(═O)OR¹⁴, C(═O)SR¹⁴, S(O)₂R¹⁴, C(═O)NR¹³R¹⁴, S(O)₂NR¹³R¹⁴, CH₂OC(═O)OR¹⁴, CH₂OC(═O)NR¹³R¹⁴ or CH₂OC(═O)R¹⁴; or propargyl, allyl or benzyl.
Embodiment 6a. A compound of Formula 1 or any one of the preceding Embodiments wherein R⁴ is H, C(═O)R¹⁴, C(═S)R¹⁴, C(═O)OR¹⁴, C(═O)SR¹⁴, S(O)₂R¹⁴, C(═O)NR¹³R¹⁴, S(O)₂NR¹³R¹⁴, CH₂OC(═O)OR¹⁴, CH₂OC(═O)NR¹³R¹⁴ or CH₂OC(═O)R¹⁴.
Embodiment 6aa. A compound of Embodiment 6 wherein R⁴ is H, C(═O)R¹⁴, CO₂R¹⁴, C(═O)SR¹⁴, S(O)₂R¹⁴, CH₂OC(═O)OR¹⁴ or CH₂OCOR¹⁴.
Embodiment 6b. A compound of Embodiment 6aa wherein R⁴ is H, SO₂CF₃, SO₂CH₃, CO₂Me, COMe, CH₂OCO-t-Bu, CH₂OCO-n-Bu, CH₂OCO-c-hexyl, CH₂OCO-c-pentyl, CH₂OCOCH₂CH₃, COMe, CH₂OCOPh, CH₂OCO-i-Bu, CH₂OCOMe, CH₂OCO-sec-Bu, CH₂OCO-n-Pr, CH₂OCO-i-Pr or (C═O)SMe.
Embodiment 6c. A compound of Embodiment 6a wherein R⁴ is H, CH₂OCOR¹⁴ or —S(O)₂R¹⁴.
Embodiment 6d. A compound of Embodiment 6c wherein R⁴ is H, CH₂OCO-t-Bu or S(O)₂CF₃.
Embodiment 6e. A compound of Embodiment 6d wherein R⁴ is H.
Embodiment 6f. A compound of Embodiment 6d wherein R⁴ is S(O)₂CF₃.
Embodiment 6g. A compound of Embodiment 6 wherein R⁴ is propargyl, allyl or benzyl.
Embodiment 6h. A compound of Embodiment 6g wherein R⁴ is benzyl.
Embodiment 6g. A compound of Embodiment 6 wherein R⁴ is propargyl.
Embodiment 6g. A compound of Embodiment 6 wherein R⁴ is allyl.
Embodiment 7. A compound of Formula 1 or any one of the preceding Embodiments wherein R⁵ is H, C₂-C₆ alkenyl, C₂-C₇ haloalkenyl, C₂-C₆ alkynyl, C₃-C₇ cycloalkyl, C₄-C₇ cycloalkylalkyl, C₂-C₇ alkoxyalkyl or C₄-C₇ alkylcycloalkyl.
Embodiment 7a. A compound of Embodiment 7 wherein R⁵ is H, C₄-C₇ cycloalkylalkyl or C₂-C₇ alkoxyalkyl; Embodiment 7b. A compound of Embodiment 7a wherein R⁵ is H.
Embodiment 8. A compound of Formula 1 or any one of the preceding Embodiments wherein R⁶ is H, C₁-C₇ alkyl, C₂-C₆ alkenyl, C₂-C₆ alkynyl, C₃-C₇ cycloalkyl, C₄-C₇ cycloalkylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy or C₄-C₇ alkylcycloalkyl.
Embodiment 8a. A compound of Embodiment 8 wherein R⁶ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy.
Embodiment 8b. A compound of Embodiment 8a wherein R⁶ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy.
Embodiment 8c. A compound of Embodiment 8b wherein R⁶ is H, C₁-C₇ alkyl or C₁-C₇ alkoxy.
Embodiment 8d. A compound of Embodiment 8b wherein R⁶ is H, Me or OMe.
Embodiment 8e. A compound of Embodiment 8d wherein R⁶ is H.
Embodiment 8f. A compound of Embodiment 8d wherein R⁶ is Me.
Embodiment 8g. A compound of Embodiment 8d wherein R⁶ is OMe.
Embodiment 9. A compound of Formula 1 or any one of the preceding Embodiments wherein R⁷ is H, C₁-C₇ alkyl, C₂-C₆ alkenyl, C₂-C₆ alkynyl, C₃-C₇ cycloalkyl, C₄-C₇ cycloalkylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy or C₄-C₇ alkylcycloalkyl.
Embodiment 9a. A compound of Embodiment 9 wherein R⁷ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy.
Embodiment 9b. A compound of Embodiment 9a wherein R⁷ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy.
Embodiment 9c. A compound of Embodiment 9b wherein R⁷ is H, C₁-C₇ alkyl or C₁-C₇ alkoxy.
Embodiment 9d. A compound of Embodiment 9b wherein R⁷ is H, Me or OMe.
Embodiment 9e. A compound of Embodiment 9d wherein R⁷ is H.
Embodiment 9f. A compound of Embodiment 8d wherein R⁷ is Me.
Embodiment 9g. A compound of Embodiment 9d wherein R⁷ is OMe.
Embodiment 10. A compound of Formula 1 or any one of the preceding Embodiments wherein R⁸ is H, C₁-C₇ alkyl, C₂-C₆ alkenyl, C₂-C₆ alkynyl, C₃-C₇ cycloalkyl, C₄-C₇ cycloalkylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ alkoxyalkyl or C₄-C₇ alkylcycloalkyl.
Embodiment 10a. A compound of Embodiment 10 wherein R⁸ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy.
Embodiment 10b. A compound of Embodiment 10a wherein R⁸ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy.
Embodiment 10c. A compound of Embodiment 10b wherein R⁸ is H, C₁-C₇ alkyl or C₁-C₇ alkoxy.
Embodiment 10d. A compound of Embodiment 10b wherein R⁸ is H, Me or OMe.
Embodiment 10e. A compound of Embodiment 10d wherein R⁸ is H.
Embodiment 10f A compound of Embodiment 10d wherein R⁸ is Me.
Embodiment 10g. A compound of Embodiment 10d wherein R⁸ is OMe.
Embodiment 11. A compound of Formula 1 or any one of the preceding Embodiments wherein R⁹ is H, C₁-C₇ alkyl, C₂-C₆ alkenyl, C₂-C₆ alkynyl, C₃-C₇ cycloalkyl, C₄-C₇ cycloalkylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy or C₄-C₇ alkylcycloalkyl.
Embodiment 11a. A compound of Embodiment 11 wherein R⁹ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy.
Embodiment 11b. A compound of Embodiment 11a wherein R⁹ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy.
Embodiment 11c. A compound of Embodiment 11b wherein R⁹ is H, C₁-C₇ alkyl or C₁-C₇ alkoxy.
Embodiment 11d. A compound of Embodiment 11b wherein R⁹ is H, Me or OMe.
Embodiment 11e. A compound of Embodiment 11d wherein R⁹ is H.
Embodiment 1 if A compound of Embodiment 11d wherein R⁹ is Me.
Embodiment 11g. A compound of Embodiment 11d wherein R⁹ is OMe.
Embodiment 12. A compound of Formula 1 or any one of the preceding Embodiments wherein G is OR¹⁰, SR¹⁰, SOR¹⁰ or SO₂R¹⁰; or G and R⁵ are taken together to form N—OR¹⁵ where R¹⁵ is H, C₁-C₆ alkyl, C₁-C₆ haloalkyl, C₂-C₆ alkenyl, C₂-C₆ alkynyl or C₄-C₇ cycloalkylalkyl.
Embodiment 12a. A compound of Embodiment 12 wherein G is OR¹⁰, SR¹⁰, SOR¹⁰ or SO₂R¹⁰.
Embodiment 12aa. A compound of Embodiment 12a wherein G is OR¹⁰ or SR¹⁰
Embodiment 12b. A compound of Embodiment 12aa wherein G is OR¹⁰.
Embodiment 12c. A compound of Embodiment 12aa wherein G is SR¹⁰.
Embodiment 12d. A compound of Embodiment 12 wherein G is SOR¹⁰.
Embodiment 12e. A compound of Embodiment 12 wherein G is SO₂R¹⁰.
Embodiment 12f. A compound of Embodiment 12 wherein G and R⁵ are attached to the same carbon ring member.
Embodiment 12g. A compound of Embodiment 12 wherein G and R⁵ are taken together to form N—OR¹⁵.
Embodiment 12gg. A compound of Embodiment 12g wherein R¹⁵ is H, C₁-C₆ alkyl, C₁-C₆ haloalkyl, C₂-C₆ alkenyl, C₂-C₆ alkynyl or C₄-C₇ cycloalkylalkyl.
Embodiment 12h. A compound of Embodiment 12g wherein R¹⁵ is H.
Embodiment 12i. A compound of Embodiment 12g wherein R¹⁵ is C₁-C₆ alkyl.
Embodiment 12j. A compound of Embodiment 12g wherein R¹⁵ is H, Me, Et, CH₂CH═CH₂ or CH₂C═CH.
Embodiment 12k. A compound of Embodiment 12j wherein R¹⁵ is Me, Et, CH₂CH═CH₂ or CH₂C═CH.
Embodiment 12l. A compound of Embodiment 12a wherein G and R⁵ are attached to the same carbon.
Embodiment 12m. A compound of Embodiment 12l wherein R⁵ is H.
Embodiment 12n. A compound of Embodiment 12a wherein G and R⁶ are attached to the same carbon.
Embodiment 12o. A compound of Embodiment 12n wherein R⁶ is H.
Embodiment 12p. A compound of Embodiment 12a wherein G and R⁷ are attached to the same carbon.
Embodiment 12q. A compound of Embodiment 12p wherein R⁷ is H.
Embodiment 12r. A compound of Embodiment 12a wherein G and R⁹ are attached to the same carbon.
Embodiment 12s. A compound of Embodiment 12r wherein R⁹ is H.
Embodiment 13. A compound of Formula 1 or any one of the preceding Embodiments wherein R¹⁰ is H, C₂-C₆ alkenyl, C₂-C₆ alkynyl, C₃-C₇ cycoalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₁-C₆ nitroalkyl, C₃-C₆ alkylcarboalkyl, C₃-C₆ alkoxycarboalkyl, C₂-C₇ haloalkoxyalkyl, benzyl or C₃-C₆ alkylcarboalkoxy; or
   R¹⁰ is selected from the group consisting of

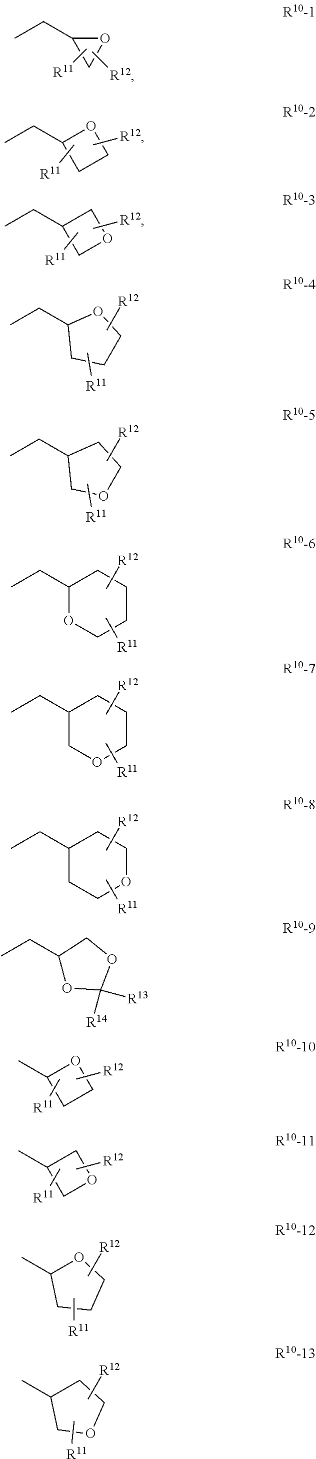

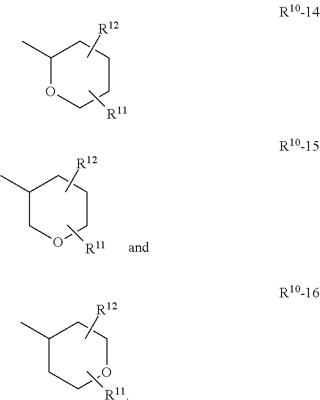

Embodiment 13a. A compound of Embodiment 13 wherein R¹⁰ is H, C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₂-C₇ haloalkoxyalkyl, benzyl or C₄-C₇ alkylcycloalkyl.
Embodiment 13aa. A compound of Embodiment 13a wherein R¹⁰ is H, C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₄ cyanoalkyl, C₁-C₇ haloalkyl, C₂-C₇ haloalkenyl, C₃-C₇ alkylthioalkyl, C₂-C₇ haloalkoxyalkyl, benzyl or C₄-C₇ alkylcycloalkyl.
Embodiment 13b. A compound of Embodiment 13aa wherein R¹⁰ is C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₂-C₄ cyanoalkyl, C₃-C₇ alkylthioalkyl, benzyl or C₄-C₇ alkylcycloalkyl.
Embodiment 13c. A compound of Embodiment 13b wherein R¹⁰ is C₂-C₆ alkenyl, C₂-C₆ alkynyl, C₃-C₇ cycloalkyl, C₄-C₇ cycloalkylalkyl, C₄-C₇ halocycloalkylalkyl or C₄-C₇ alkylcycloalkyl.
Embodiment 13d. A compound of Embodiment 13c wherein R¹⁰ is C₂-C₆ alkenyl, C₂-C₆ alkynyl, C₃-C₇ cycloalkyl or C₄-C₇ halocycloalkylalkyl.
Embodiment 13dd. A compound of Embodiment 13d wherein R¹⁰ is C₂-C₆ alkenyl, C₂-C₆ alkynyl or C₃-C₇ cycloalkyl.
Embodiment 13e. A compound of Embodiment 13d wherein R¹⁰ is cyclopropyl, cyclobutyl, cyclopentyl, allyl or propargyl.
Embodiment 13ee. A compound of Embodiment 13e wherein R¹⁰ is H.
Embodiment 13f. A compound of Embodiment 13e wherein R¹⁰ is cyclopropyl.
Embodiment 13g. A compound of Embodiment 13e wherein R¹⁰ is cyclobutyl.
Embodiment 13gg. A compound of Embodiment 13e wherein R¹⁰ is cyclopentyl.
Embodiment 13ggg. A compound of Embodiment 13e wherein R¹⁰ is cyclohexyl.
Embodiment 13h. A compound of Embodiment 13e wherein R¹⁰ is allyl.
Embodiment 13i. A compound of Embodiment 13e wherein R¹⁰ is propargyl.
Embodiment 13j. A compound of Embodiment 13 wherein R¹⁰ is R¹⁰-1, R¹⁰-2, R¹⁰-3, R¹⁰-4, R¹⁰-5, R¹⁰-6, R¹⁰-7, R¹⁰-8, R¹⁰-9, R¹⁰-10, R¹⁰-11, R¹⁰-12, R¹⁰-13, R¹⁰-14, R¹⁰-15 or R¹⁰-16.
Embodiment 13k. A compound of Embodiment 13j wherein R¹⁰ is R¹⁰-1, R¹⁰-2, R¹⁰-3, R¹⁰-4, R¹⁰-5, R¹⁰-6, R¹⁰-7, R¹⁰-8 or R¹⁰-9.
Embodiment 13l. A compound of Embodiment 13k wherein R¹⁰ is R¹⁰-3 or R¹⁰-4.
Embodiment 13m. A compound of Embodiment 13a wherein R¹⁰ is C₂-C₆ alkenyl, C₂-C₆ alkynyl, C₃-C₇ cycloalkyl, C₄-C₇ halocycloalkylalkyl, C₄-C₇ cycloalkylalkyl or benzyl.
Embodiment 14. A compound of Formula 1 or any one of the preceding Embodiments wherein R¹¹ is H or C₁-C₇ alkyl.
Embodiment 14a. A compound of Formula 1 or any one of the preceding Embodiments wherein R¹¹ is H.
Embodiment 15. A compound of Formula 1 or any one of the preceding Embodiments wherein R¹² is H or C₁-C₇ alkyl.
Embodiment 15a. A compound of Formula 1 or any one of the preceding Embodiments wherein R¹² is H.
Embodiment 16. A compound of Formula 1 or any one of the preceding Embodiments wherein each R¹³ and R¹⁴ is independently H, C₁-C₇ haloalkyl or C₁-C₇ alkyl.
Embodiment 16a. A compound of Embodiment 16 wherein each R¹³ and R¹⁴ is independently C₁-C₄ alkyl.
Embodiment 16b. A compound of Embodiment 16a wherein each R¹³ and R¹⁴ is independently C₁-C₃ haloalkyl.
Embodiment 16c. A compound of Embodiment 16 wherein each R¹³ and R¹⁴ is independently CF₃.
Embodiment 17. A compound of Formula 1 or any one of the preceding Embodiments wherein R^{f} is C₁-C₃ haloalkyl.
Embodiment 17a. A compound of Embodiment 28 wherein R^{f} is CF₃.

Embodiments of this invention, including Embodiments 1-17a above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-17a above as well as any other embodiments described herein, and any combination thereof, pertain to the compositions and methods of the present invention.

Combinations of Embodiments 1-17a are illustrated by:
Embodiment A. A compound of Formula 1 as described in the Summary of the Disclosure wherein
   Q is direct bond;
   R¹ is H, C₁-C₇ alkyl, halogen, C₂-C₆ alkenyl, C₂-C₆ alkynyl, C₃-C₇ cycloalkyl, C₁-C₇ haloalkyl;
   R² is H, C₁-C₇ alkyl, halogen or —CN;
   R³ is H, C₁-C₇ alkyl, halogen, CN, C₁-C₇ alkoxy or C₁-C₇ haloalkyl;
   R⁴ is H, —C(═O)R¹⁴, —C(═S)R¹⁴, —CO₂R¹⁴, —C(═O)SR¹⁴, —S(O)₂R¹⁴, —C(═O)NR¹³R¹⁴, —S(O)₂NR¹³R¹⁴, —CH₂OC(═O)OR¹⁴, —CH₂OC(═O)NR¹³R¹⁴ or —CH₂OC(═O)R¹⁴;
   R⁵ is H, C₂-C₆ alkenyl, C₂-C₇ haloalkenyl, C₂-C₆ alkynyl, C₃-C₇ cycloalkyl, C₄-C₇ cycloalkylalkyl, C₂-C₇ alkoxyalkyl or C₄-C₇ alkylcycloalkyl;
   R⁶ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R⁷ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   R⁸ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   G is OR¹⁰, SR¹⁰, SOR¹⁰ or SO₂R¹⁰;
   R¹⁰ is H, C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₂-C₇ haloalkoxyalkyl, benzyl or C₄-C₇ alkylcycloalkyl;
   R¹¹ is H or C₁-C₇ alkyl;
   R¹² is H or C₁-C₇ alkyl;
   each R¹³ and R¹⁴ is independently H, C₁-C₇ haloalkyl or C₁-C₇ alkyl; and
   R^{f} is C₁-C₃ haloalkyl.
Embodiment A1. A compound of Embodiment A wherein
   R¹ is H, C₁-C₃ alkyl, halogen or C₃-C₄ cycloalkyl;
   R² is H, Me, F, Cl or CN;
   R³ is H, Me, F, Cl, CN, OMe or CF₃;
   R⁴ is H, SO₂CF₃, SO₂CH₃, CO₂Me, COMe, CH₂OCO-t-Bu, CH₂OCO-n-Bu, CH₂OCO-c-hexyl, CH₂OCO-c-pentyl, CH₂OCOCH₂CH₃, COMe, CH₂OCOPh, CH₂OCO-i-Bu, CH₂OCOMe, CH₂OCO-sec-Bu, CH₂OCO-n-Pr and CH₂OCO-i-Pr or (C═O)SMe;
   R⁵ is H, C₄-C₇ cycloalkylalkyl or C₂-C₇ alkoxyalkyl;
   R⁶ is H, C₁-C₇ alkyl or C₁-C₇ alkoxy;
   R⁷ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R⁸ is H, C₁-C₇ alkyl or C₁-C₇ alkoxy;
   G is OR¹⁰ or SR¹⁰; and
   R¹⁰ is C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₂-C₄ cyanoalkyl, C₃-C₇ alkylthioalkyl, benzyl or C₄-C₇ alkylcycloalkyl.
Embodiment A2. A compound of Embodiment A1 wherein
   R¹ is H, Me, halogen or cyclopropyl;
   R² is H or F;
   R³ is Me or F;
   R⁴ is H, CH₂OCOR¹⁴ or —S(O)₂R¹⁴;
   R⁵ is H;
   R⁶ is H, Me or OMe;
   R⁷ is H, Me or OMe;
   R⁸ is H, Me or OMe;
   G is OR¹⁰; and
   R¹⁰ is C₂-C₆ alkenyl, C₂-C₆ alkynyl, C₃-C₇ cycloalkyl, C₄-C₇ cycloalkylalkyl, C₄-C₇ halocycloalkylalkyl or C₄-C₇ alkylcycloalkyl.
Embodiment A3. A compound of Embodiment A2 wherein
   R¹ is H, Me, F, Cl, Br or cyclopropyl;
   R⁴ is H, CH₂OCO-t-Bu or SO₂CF₃;
   R⁸ is H; and
   R¹⁰ is C₂-C₆ alkenyl, C₂-C₆ alkynyl or C₃-C₇ cycloalkyl.
Embodiment A4. A compound of Embodiment A3 wherein
   R¹ is Me;
   R³ is Me;
   R⁴ is H;
   R⁶ is H;
   R⁷ is H; and
   R¹⁰ is cyclopropyl, cyclobutyl, cyclopentyl, allyl or propargyl.
Embodiment B. A compound of Formula 1 as described in the Summary of the Disclosure wherein
   Q is CHR⁹;
   R¹ is H, C₁-C₇ alkyl, halogen, C₂-C₆ alkenyl, C₂-C₆ alkynyl, C₃-C₇ cycloalkyl, C₁-C₇ haloalkyl;
   R² is H, C₁-C₇ alkyl, halogen or CN;
   R³ is H, C₁-C₇ alkyl, halogen, CN, C₁-C₇ alkoxy or C₁-C₇ haloalkyl;
   R⁴ is H, C(═O)R¹⁴, —C(═S)R¹⁴, —CO₂R¹⁴, —C(═O)SR¹⁴, —S(O)₂R¹⁴, C(═O)NR¹³R¹⁴, —S(O)₂NR¹³R¹⁴, CH₂OC(═O)OR¹⁴, CH₂OC(═O)NR¹³R¹⁴ or CH₂OC(═O)R¹⁴;
   R⁵ is H, C₂-C₆ alkenyl, C₂-C₇ haloalkenyl, C₂-C₆ alkynyl, C₃-C₇ cycloalkyl, C₄-C₇ cycloalkylalkyl, C₂-C₇ alkoxyalkyl or C₄-C₇ alkylcycloalkyl;
   R⁶ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R⁷ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   R⁸ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   G is OR¹⁰, SR¹⁰, SOR¹⁰ or SO₂R¹⁰;
   R⁹ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R¹⁰ is H, C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₂-C₇ haloalkoxyalkyl, benzyl or C₄-C₇ alkylcycloalkyl;
   R¹¹ is H or C₁-C₇ alkyl;
   R¹² is H or C₁-C₇ alkyl;
   each R¹³ and R¹⁴ is independently H, C₁-C₇ haloalkyl or C₁-C₇ alkyl; and
   R^{f} is C₁-C₃ haloalkyl.
Embodiment B1. A compound of Embodiment B wherein
   R¹ is H, C₁-C₃ alkyl, halogen or C₃-C₄ cycloalkyl;
   R² is H, Me, F, Cl or CN;
   R³ is H, Me, F, Cl, —CN, OMe or CF₃;
   R⁴ is H, SO₂CF₃, SO₂CH₃, CO₂Me, COMe, CH₂OCO-t-Bu, CH₂OCO-n-Bu, CH₂OCO-c-hexyl, CH₂OCO-c-pentyl, CH₂OCOCH₂CH₃, COMe, CH₂OCOPh, CH₂OCO-i-Bu, CH₂OCOMe, CH₂OCO-sec-Bu, CH₂OCO-n-Pr and CH₂OCO-i-Pr or (C═O)SMe;
   R⁵ is H, C₄-C₇ cycloalkylalkyl or C₂-C₇ alkoxyalkyl;
   R⁶ is H, C₁-C₇ alkyl or C₁-C₇ alkoxy;
   R⁷ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R⁸ is H, C₁-C₇ alkyl or C₁-C₇ alkoxy;
   G is OR¹⁰ or SR¹⁰;
   R⁹ is H, C₁-C₇ alkyl or C₁-C₇ alkoxy; and
   R¹⁰ is C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₂-C₄ cyanoalkyl, C₃-C₇ alkylthioalkyl or C₄-C₇ alkylcycloalkyl.
Embodiment B2. A compound of Embodiment B1 wherein
   R¹ is H, Me, halogen or cyclopropyl;
   R² is H or F;
   R³ is Me or F;
   R⁴ is H, CH₂OCOR¹⁴ or —S(O)₂R¹⁴;
   R⁵ is H;
   R⁶ is H, Me or OMe;
   R⁷ is H, Me or OMe;
   R⁸ is H, Me or OMe;
   G is OR¹⁰;
   R⁹ is H, Me or OMe; and
   R¹⁰ is H, C₂-C₆ alkenyl, C₂-C₆ alkynyl, C₃-C₇ cycloalkyl, C₄-C₇ cycloalkylalkyl, C₄-C₇ halocycloalkylalkyl or C₄-C₇ alkylcycloalkyl.
Embodiment B3. A compound of Embodiment B2 wherein
   R¹ is H, Me, F, Cl, Br or cyclopropyl;
   R⁴ is H, CH₂OCO-t-Bu or SO₂CF₃;
   R⁸ is H;
   R⁹ is H; and
   R¹⁰ is C₂-C₆ alkenyl, C₂-C₆ alkynyl or C₃-C₇ cycloalkyl
Embodiment C. A compound of Formula 1 as described in the Summary of the Disclosure wherein
   Q is O;
   R¹ is H, C₁-C₇ alkyl, halogen, C₂-C₆ alkenyl, C₂-C₆ alkynyl, C₃-C₇ cycloalkyl, C₁-C₇ haloalkyl;
   R² is H, C₁-C₇ alkyl, halogen or CN;
   R³ is H, C₁-C₇ alkyl, halogen, CN, C₁-C₇ alkoxy or C₁-C₇ haloalkyl;
   R⁴ is H, C(═O)R¹⁴, —C(═S)R¹⁴, —CO₂R¹⁴, —C(═O)SR¹⁴, —S(O)₂R¹⁴, C(═O)NR¹³R¹⁴, —S(O)₂NR¹³R¹⁴, CH₂OC(═O)OR¹⁴, CH₂OC(═O)NR¹³R¹⁴ or CH₂OC(═O)R¹⁴;
   R⁵ is H, C₂-C₆ alkenyl, C₂-C₇ haloalkenyl, C₂-C₆ alkynyl, C₃-C₇ cycloalkyl, C₄-C₇ cycloalkylalkyl, C₂-C₇ alkoxyalkyl or C₄-C₇ alkylcycloalkyl;
   R⁶ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R⁷ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   R⁸ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   G is OR¹⁰, SR¹⁰, SOR¹⁰ or SO₂R¹⁰;
   R¹⁰ is H, C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₂-C₇ haloalkoxyalkyl, benzyl or C₄-C₇ alkylcycloalkyl;
   R¹¹ is H or C₁-C₇ alkyl;
   R¹² is H or C₁-C₇ alkyl;
   each R¹³ and R¹⁴ is independently H, C₁-C₇ haloalkyl or C₁-C₇ alkyl; and
   R^{f} is C₁-C₃ haloalkyl.
Embodiment C1. A compound of Embodiment C wherein
   R¹ is H, C₁-C₃ alkyl, halogen or C₃-C₄ cycloalkyl;
   R² is H, Me, F, Cl or CN;
   R³ is H, Me, F, Cl, —CN, OMe or CF₃;
   R⁴ is H, SO₂CF₃, SO₂CH₃, CO₂Me, COMe, CH₂OCO-t-Bu, CH₂OCO-n-Bu, CH₂OCO-c-hexyl, CH₂OCO-c-pentyl, CH₂OCOCH₂CH₃, COMe, CH₂OCOPh, CH₂OCO-i-Bu, CH₂OCOMe, CH₂OCO-sec-Bu, CH₂OCO-n-Pr and CH₂OCO-i-Pr or (C═O)SMe;
   R⁵ is H, C₄-C₇ cycloalkylalkyl or C₂-C₇ alkoxyalkyl;
   R⁶ is H, C₁-C₇ alkyl or C₁-C₇ alkoxy;
   R⁷ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R⁸ is H, C₁-C₇ alkyl or C₁-C₇ alkoxy;
   G is OR¹⁰ or SR¹⁰; and
   R¹⁰ is C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₂-C₄ cyanoalkyl, C₃-C₇ alkylthioalkyl, benzyl or C₄-C₇ alkylcycloalkyl.
Embodiment C2. A compound of Embodiment C1 wherein
   R¹ is H, Me, halogen or cyclopropyl;
   R² is H or F;
   R³ is Me or F;
   R⁴ is H, CH₂OCOR¹⁴ or —S(O)₂R¹⁴;
   R⁵ is H;
   R⁶ is H, Me or OMe;
   R⁷ is H, Me or OMe;
   R⁸ is H, Me or OMe;
   G is OR¹⁰; and
   R¹⁰ is C₂-C₆ alkenyl, C₂-C₆ alkynyl, C₃-C₇ cycloalkyl, C₄-C₇ cycloalkylalkyl, C₄-C₇ halocycloalkylalkyl or C₄-C₇ alkylcycloalkyl.
Embodiment C3. A compound of Embodiment C2 wherein
   R¹ is H, Me, F, Cl, Br or cyclopropyl;
   R⁴ is H, CH₂OCO-t-Bu or S(O)₂CF₃;
   R⁸ is H; and
   R¹⁰ is C₂-C₆ alkenyl, C₂-C₆ alkynyl or C₃-C₇ cycloalkyl.
Embodiment C4. A compound of Embodiment C3 wherein
   R¹ is Me;
   R³ is Me;
   R⁴ is H;
   R⁶ is H;
   R⁷ is H; and
   R¹⁰ is cyclopropyl, cyclobutyl, cyclopentyl, allyl or propargyl.
Embodiment D. A compound of Formula 1 as described in the Summary of the Disclosure wherein
   R¹ is H, C₁-C₇ alkyl, halogen, C₂-C₆ alkenyl, C₂-C₆ alkynyl, C₃-C₇ cycloalkyl, C₁-C₇ haloalkyl;
   R² is H, C₁-C₇ alkyl, halogen or CN;
   R³ is H, C₁-C₇ alkyl, halogen, CN, C₁-C₇ alkoxy or C₁-C₇ haloalkyl;
   R⁴ is H, C(═O)R¹⁴, —C(═S)R¹⁴, —CO₂R¹⁴, —C(═O)SR¹⁴, —S(O)₂R¹⁴, C(═O)NR¹³R¹⁴, —S(O)₂NR¹³R¹⁴, CH₂OC(═O)OR¹⁴, CH₂OC(═O)NR¹³R¹⁴ or CH₂OC(═O)R¹⁴;
   R⁶ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R⁷ is H, C₁-C₇ alkyl, C₂-C₆ alkenyl, C₂-C₆ alkynyl, C₃-C₇ cycloalkyl, C₃-C₇ alkenylalkyl, C₃-C₇ alkynylalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   R⁸ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   G and R⁵ are taken together to form N—OR¹⁵;
   R¹¹ is H or C₁-C₇ alkyl;
   R¹² is H or C₁-C₇ alkyl;
   R¹³ and R¹⁴ are independently H, C₁-C₇ haloalkyl or C₁-C₇ alkyl;
   R^{f} is C₁-C₃ haloalkyl; and
   R¹⁵ is H, C₁-C₆ alkyl, C₁-C₆ haloalkyl, C₂-C₆ alkenyl, C₂-C₆ alkynyl or C₄-C₇ cycloalkylalkyl.
Embodiment D1. A compound of Embodiment D wherein
   R¹ is H, C₁-C₃ alkyl, halogen or C₃-C₄ cycloalkyl;
   R² is H, Me, F, Cl or CN;
   R³ is H, Me, F, Cl, —CN, OMe or CF₃;
   R⁴ is H, SO₂CF₃, SO₂CH₃, CO₂Me, COMe, CH₂OCO-t-Bu, CH₂OCO-n-Bu, CH₂OCO-c-hexyl, CH₂OCO-c-pentyl, CH₂OCOCH₂CH₃, COMe, CH₂OCOPh, CH₂OCO-i-Bu, CH₂OCOMe, CH₂OCO-sec-Bu, CH₂OCO-n-Pr and CH₂OCO-i-Pr or (C═O)SMe;
   R⁶ is H, C₁-C₇ alkyl or C₁-C₇ alkoxy;
   R⁷ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy; and
   R⁸ is H, C₁-C₇ alkyl or C₁-C₇ alkoxy.
Embodiment D2. A compound of Embodiment D1 wherein
   R¹ is H, Me, halogen or cyclopropyl;
   R² is H or F;
   R³ is Me or F;
   R⁴ is H, CH₂OCOR¹⁴ or —S(O)₂R¹⁴;
   R⁶ is H, Me or OMe;
   R⁷ is H, Me or OMe; and
   R⁸ is H, Me or OMe.
Embodiment D3. A compound of Embodiment D2 wherein
   R¹ is H, Me, F, Cl, Br or cyclopropyl;
   R⁴ is H, CH₂OCO-t-Bu or SO₂CF₃; and
   R⁸ is H.
Embodiment D4. A compound of Embodiment D3 wherein
   R¹ is Me;
   R³ is Me;
   R⁴ is H;
   R⁶ is H;
   R⁷ is H; and
   R¹⁵ is H, Me, Et, CH₂CH═CH₂ or CH₂C═CH.
Embodiment D5. A compound of any one of Embodiments D to D4 wherein
   Q is direct bond.
Embodiment P1. A compound selected from Formula 1, all stereoisomers, N-oxides, and salts thereof,

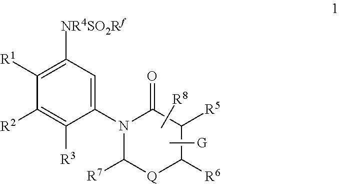

wherein
   R¹ is H, C₁-C₇ alkyl, halogen, CN, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₃-C₇ haloalkynyl, C₂-C₇ alkoxyalkyl, C₁-C₇ alkoxy, C₁-C₅ alkylthio, C₂-C₃ alkoxycarbonyl or C₂-C₇ haloalkoxyalkyl;
   R² is H, C₁-C₇ alkyl, halogen, CN, C₁-C₇ haloalkyl, C₁-C₇ alkoxy or C₁-C₅ alkylthio;
   R³ is H, C₁-C₇ alkyl, halogen, CN, C₂-C₆ alkenyl, C₂-C₇ alkynyl, C₃-C₇ cycloalkyl, C₂-C₄ cyanoalkyl, C₁-C₇ haloalkyl, C₂-C₇ haloalkenyl, C₃-C₇ haloalkynyl, C₂-C₇ alkoxyalkyl, C₁-C₇ alkoxy, C₁-C₅ alkylthio, C₂-C₃ alkoxycarbonyl or C₂-C₇ haloalkoxyalkyl;
   R⁴ is H, C(═O)R¹⁴, —C(═S)R¹⁴, —CO₂R¹⁴, —C(═O)SR¹⁴, —S(O)₂R¹⁴, C(═O)NR¹³R¹⁴, —S(O)₂NR¹³R¹⁴, CH₂OC(═O)OR¹⁴, CH₂OC(═O)NR¹³R¹⁴ or CH₂OC(═O)R¹⁴; or propargyl, allyl or benzyl;
   R⁵ is H, C₂-C₆ alkenyl, C₂-C₇ haloalkenyl, C₂-C₆ alkynyl, C₃-C₇ cycloalkyl, C₄-C₇ cycloalkylalkyl, C₃-C₇ alkenylalkyl, C₃-C₇ alkynylalkyl, C₂-C₄ cyanoalkyl, C₁-C₇ haloalkyl, C₃-C₇ alkylthioalkyl, C₁-C₇ haloalkoxy, C₂-C₇ alkoxyalkyl or C₄-C₇ alkylcycloalkyl;
   R⁶ is H, C₁-C₇ alkyl, halogen, CN, C₁-C₅ alkylthio, C₂-C₃ alkoxycarbonyl, C₂-C₆ alkenyl, C₂-C₆ alkynyl, C₃-C₇ cycloalkyl, C₄-C₇ cycloalkylalkyl, C₃-C₇ alkenylalkyl, C₃-C₇ alkynylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ haloalkoxyalkyl or C₄-C₇ alkylcycloalkyl;
   R⁷ is H, C₁-C₇ alkyl, halogen, CN, C₁-C₅ alkylthio, C₂-C₃ alkoxycarbonyl, C₂-C₆ alkenyl, C₂-C₆ alkynyl, C₃-C₇ cycloalkyl, C₄-C₇ cycloalkylalkyl, C₃-C₇ alkenylalkyl, C₃-C₇ alkynylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ haloalkoxyalkyl or C₄-C₇ alkylcycloalkyl;
   R⁸ is H, C₁-C₇ alkyl, C₂-C₆ alkenyl, C₂-C₆ alkynyl, C₃-C₇ cycloalkyl, C₄-C₇ cycloalkylalkyl, C₃-C₇ alkenylalkyl, C₃-C₇ alkynylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ alkoxyalkyl or C₄-C₇ alkylcycloalkyl;
   Q is CHR⁹, O or a direct bond;
   R⁹ is H, C₁-C₇ alkyl, halogen, CN, C₁-C₅ alkylthio, C₂-C₃ alkoxycarbonyl, C₂-C₆ alkenyl, C₂-C₆ alkynyl, C₃-C₇ cycloalkyl, C₄-C₇ cycloalkylalkyl, C₃-C₇ alkenylalkyl, C₃-C₇ alkynylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ alkoxyalkyl, C₂-C₇ haloalkoxyalkyl or C₄-C₇ alkylcycloalkyl;
   G is OR¹⁰, SR¹⁰, SOR¹⁰ or SO₂R¹⁰;
   R¹⁰ is H, C₂-C₆ alkenyl, C₂-C₆ alkynyl, C₃-C₇ cycloalkyl, C₃-C₇ halocycloalkyl, C₃-C₁₀ alkenylalkyl, C₃-C₁₀ alkynylalkyl, C₄-C₁₀ alkylalkenylalkyl, C₄-C₁₀ alkylalkynylalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₂-C₄ cyanoalkyl, C₄-C₇ alkylcycloalkyl, C₁-C₆ nitroalkyl, C₃-C₆ alkylcarboalkyl, C₃-C₆ alkoxycarboalkyl or C₃-C₆ alkylcarboalkoxy; or
   R¹⁰ is selected from the group consisting of

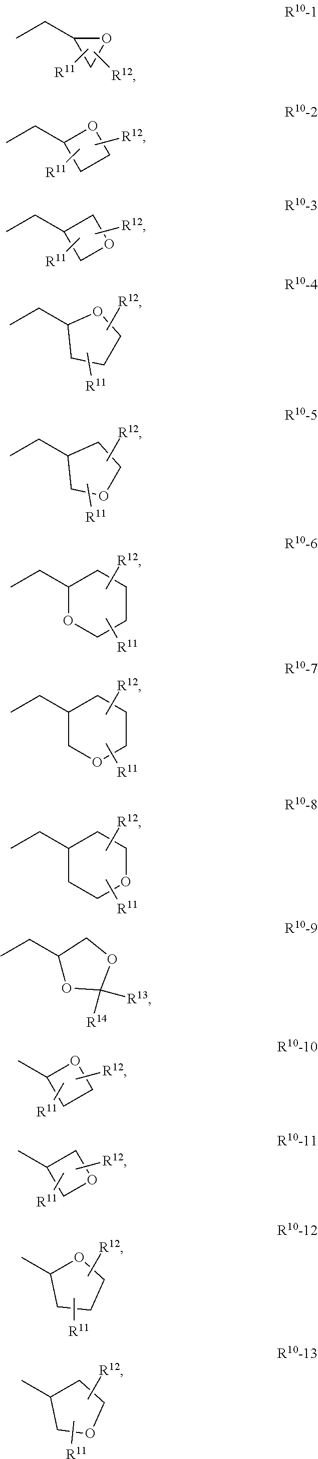

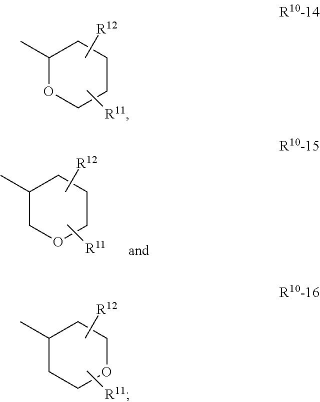

R¹¹ is H, C₁-C₇ alkyl, C₂-C₆ alkenyl, C₂-C₆ alkynyl, C₃-C₇ cycloalkyl, C₄-C₇ cycloalkylalkyl, C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ alkoxyalkyl or C₄-C₇ alkylcycloalkyl;
R¹² is H, C₁-C₇ alkyl, C₂-C₆ alkenyl, C₂-C₆ alkynyl, C₃-C₇ cycloalkyl, C₄-C₇ cycloalkylalkyl or C₇ haloalkyl;
each R¹³ and R¹⁴ are independently H, C₁-C₇ alkyl, C₂-C₆ alkenyl, C₂-C₆ alkynyl, C₃-C₇ cycloalkyl, C₄-C₇ cycloalkylalkyl, C₂-C₃ cyanoalkyl, C₁-C₇ haloalkyl, C₂-C₇ haloalkenyl, C₂-C₇ alkylalkoxyalkyl, C₃-C₇ alkylthioalkyl, C₁-C₇ alkoxy; C₂-C₇ alkoxyalkyl, C₄-C₇ alkylcycloalkyl, Ph or benzyl;
R^{f} is C₁-C₇ haloalkyl;
G and R⁸ can be attached to any ring carbon(s) with available valency, said ring is the cyclic amide ring shown in Formula 1; and
each R¹¹ or R¹² can be attached to any ring carbon(s) with available valency, said ring is illustrated in R¹⁰-1 through R¹⁰-16 as above.
Embodiment P2. The compound of Embodiment P1 wherein
   Q is direct bond;
   R¹ is H, C₁-C₇ alkyl, halogen, C₂-C₆ alkenyl, C₂-C₆ alkynyl, C₃-C₇ cycloalkyl, C₁-C₇ haloalkyl;
   R² is H, C₁-C₇ alkyl, halogen or CN;
   R³ is H, C₁-C₇ alkyl, halogen, CN, C₁-C₇ alkoxy or C₁-C₇ haloalkyl;
   R⁴ is H, C(═O)R¹⁴, —C(═S)R¹⁴, —CO₂R¹⁴, —C(═O)SR¹⁴, —S(O)₂R¹⁴, C(═O)NR¹³R¹⁴, —S(O)₂NR¹³R¹⁴, CH₂OC(═O)OR¹⁴, CH₂OC(═O)NR¹³R¹⁴ or CH₂OC(═O)R¹⁴;
   R⁵ is H, C₂-C₆ alkenyl, C₂-C₇ haloalkenyl, C₂-C₆ alkynyl, C₃-C₇ cycloalkyl, C₄-C₇ cycloalkylalkyl, C₃-C₇ alkenylalkyl, C₃-C₇ alkynylalkyl, C₂-C₇ alkoxyalkyl or C₄-C₇ alkylcycloalkyl;
   R⁶ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R⁷ is H, C₁-C₇ alkyl, C₂-C₆ alkenyl, C₂-C₆ alkynyl, C₃-C₇ cycloalkyl, C₃-C₇ alkenylalkyl, C₃-C₇ alkynylalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   R⁸ is H, C₁-C₇ alkyl, C₂-C₆ alkenyl, C₂-C₆ alkynyl, C₃-C₇ cycloalkyl, C₃-C₇ alkenylalkyl, C₃-C₇ alkynylalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   G is OR¹⁰, SR¹⁰, SOR¹⁰ or SO₂R¹⁰;
   R¹⁰ is H, C₂-C₆ alkenyl, C₂-C₆ alkynyl, C₃-C₇ cycloalkyl, C₃-C₇ halocycloalkyl, C₃-C₁₀ alkenylalkyl, C₃-C₁₀ alkynylalkyl, C₄-C₁₀ alkylalkenylalkyl, C₄-C₁₀ alkylalkynylalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₂-C₇ alkoxyalkyl, C₂-C₄ cyanoalkyl or C₄-C₇ alkylcycloalkyl;
   R¹¹ is H or C₁-C₇ alkyl;
   R¹² is H or C₁-C₇ alkyl;
   R¹³ and R¹⁴ are independently H, C₁-C₇ haloalkyl or C₁-C₇ alkyl; and
   R^{f} is C₁-C₃ haloalkyl.
Embodiment P3. The compound of Embodiment P2 wherein
   R¹ is H, C₁-C₃ alkyl, halogen or C₃-C₄ cycloalkyl;
   R² is H, Me, F, Cl or CN;
   R³ is H, Me, F, Cl, —CN, OMe or CF₃;
   R⁴ is H, SO₂CF₃, SO₂CH₃, CO₂Me, COMe, CH₂OCO-t-Bu, CH₂OCO-n-Bu, CH₂OCO-c-hexyl, CH₂OCO-c-pentyl, CH₂OCOCH₂CH₃, COMe, CH₂OCOPh, CH₂OCO-i-Bu, CH₂OCOMe, CH₂OCO-sec-Bu, CH₂OCO-n-Pr and CH₂OCO-i-Pr or (C═O)SMe;
   R⁵ is H, C₄-C₇ cycloalkylalkyl or C₂-C₇ alkoxyalkyl;
   R⁶ is H, C₁-C₇ alkyl or C₁-C₇ alkoxy;
   R⁷ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R⁸ is H, C₁-C₇ alkyl or C₁-C₇ alkoxy;
   G is OR¹⁰ or SR¹⁰; and
   R¹⁰ is C₃-C₇ cycloalkyl, C₃-C₇ halocycloalkyl, C₃-C₁₀ alkenylalkyl, C₃-C₁₀ alkynylalkyl, C₄-C₁₀ alkylalkenylalkyl, C₄-C₁₀ alkylalkynylalkyl, C₄-C₇ alkylcycloalkyl, C₄-C₇ cycloalkylalkyl, C₄-C₇ halocycloalkylalkyl, C₅-C₇ alkylcycloalkylalkyl, C₂-C₇ alkoxyalkyl, C₂-C₄ cyanoalkyl, C₃-C₇ alkylthioalkyl or C₄-C₇ alkylcycloalkyl.
Embodiment P4. The compound of Embodiment P3 wherein
   R¹ is H, Me, halogen or cyclopropyl;
   R² is H or F;
   R³ is Me or F;
   R⁴ is H, CH₂OCOR¹⁴ or —S(O)₂R¹⁴;
   R⁵ is H;
   R⁶ is H, Me or OMe;
   R⁷ is H, Me or OMe;
   R⁸ is H, Me or OMe;
   G is OR¹⁰;
   R¹⁰ is C₃-C₇ cycloalkyl, C₃-C₁₀ alkenylalkyl, C₃-C₁₀ alkynylalkyl, C₄-C₇ cycloalkylalkyl, C₂-C₇ alkoxyalkyl or C₄-C₇ alkylcycloalkyl.
Embodiment P5. The compound of Embodiment P4 wherein
   R¹ is H, Me, F, Cl, Br or cyclopropyl;
   R⁴ is H, CH₂OCO-t-Bu or SO₂CF₃;
   R⁸ is H; and
   R¹⁰ is C₃-C₇ cycloalkyl, C₃-C₁₀ alkenylalkyl or C₃-C₁₀ alkynylalkyl.
Embodiment P6. The compound of Embodiment P5 wherein
   R¹ is Me;
   R³ is Me;
   R⁴ is H;
   R⁶ is H;
   R⁷ is H; and
   R¹⁰ is cyclopropyl, cyclobutyl, cyclopentyl, allyl or propargyl.
Embodiment P7. The compound of Embodiment P6 wherein
   Q is CHR⁹;
   R¹ is H, C₁-C₇ alkyl, halogen, C₂-C₆ alkenyl, C₂-C₆ alkynyl, C₃-C₇ cycloalkyl, C₁-C₇ haloalkyl;
   R² is H, C₁-C₇ alkyl, halogen or CN;
   R³ is H, C₁-C₇ alkyl, halogen, CN, C₁-C₇ alkoxy or C₁-C₇ haloalkyl;
   R⁴ is H, C(═O)R¹⁴, —C(═S)R¹⁴, —CO₂R¹⁴, —C(═O)SR¹⁴, —S(O)₂R¹⁴, C(═O)NR¹³R¹⁴, —S(O)₂NR¹³R¹⁴, CH₂OC(═O)OR¹⁴, CH₂OC(═O)NR¹³R¹⁴ or CH₂OC(═O)R¹⁴;
   R⁵ is H, C₂-C₆ alkenyl, C₂-C₇ haloalkenyl, C₂-C₆ alkynyl, C₃-C₇ cycloalkyl, C₄-C₇ cycloalkylalkyl, C₃-C₇ alkenylalkyl, C₃-C₇ alkynylalkyl, C₂-C₇ alkoxyalkyl or C₄-C₇ alkylcycloalkyl;
   R⁶ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R⁷ is H, C₁-C₇ alkyl, C₂-C₆ alkenyl, C₂-C₆ alkynyl, C₃-C₇ cycloalkyl, C₃-C₇ alkenylalkyl, C₃-C₇ alkynylalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   R⁸ is H, C₁-C₇ alkyl, C₂-C₆ alkenyl, C₂-C₆ alkynyl, C₃-C₇ cycloalkyl, C₃-C₇ alkenylalkyl, C₃-C₇ alkynylalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   G is OR¹⁰, SR¹⁰, SOR¹⁰ or SO₂R¹⁰;
   R⁹ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R¹⁰ is alkynylalkyl, C₄-C₁₀ alkylalkenylalkyl, C₄-C₁₀ alkylalkynylalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₂-C₇ alkoxyalkyl, C₂-C₄ cyanoalkyl or C₄-C₇ alkylcycloalkyl;
   R¹¹ is H or C₁-C₇ alkyl;
   R¹² is H or C₁-C₇ alkyl;
   R¹³ and R¹⁴ are independently H, C₁-C₇ haloalkyl or C₁-C₇ alkyl; and
   R^{f} is C₁-C₃ haloalkyl.
Embodiment P8. The compound of Embodiment P7 wherein
   R¹ is H, C₁-C₃ alkyl, halogen or C₃-C₄ cycloalkyl;
   R² is H, Me, F, Cl or CN;
   R³ is H, Me, F, Cl, —CN, OMe or CF₃;
   R⁴ is H, SO₂CF₃, SO₂CH₃, CO₂Me, COMe, CH₂OCO-t-Bu, CH₂OCO-n-Bu, CH₂OCO-c-hexyl, CH₂OCO-c-pentyl, CH₂OCOCH₂CH₃, COMe, CH₂OCOPh, CH₂OCO-i-Bu, CH₂OCOMe, CH₂OCO-sec-Bu, CH₂OCO-n-Pr and CH₂OCO-i-Pr or (C═O)SMe;
   R⁵ is H, C₄-C₇ cycloalkylalkyl or C₂-C₇ alkoxyalkyl;
   R⁶ is H, C₁-C₇ alkyl or C₁-C₇ alkoxy;
   R⁷ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R⁸ is H, C₁-C₇ alkyl or C₁-C₇ alkoxy;
   G is OR¹⁰ or SR¹⁰;
   R⁹ is H, C₁-C₇ alkyl or C₁-C₇ alkoxy;
   R¹⁰ is C₃-C₇ cycloalkyl, C₃-C₇ halocycloalkyl, C₃-C₁₀ alkenylalkyl, C₃-C₁₀ alkynylalkyl, C₄-C₁₀ alkylalkenylalkyl, C₄-C₁₀ alkylalkynylalkyl, C₄-C₇ alkylcycloalkyl, C₄-C₇ cycloalkylalkyl, C₄-C₇ halocycloalkylalkyl, C₅-C₇ alkylcycloalkylalkyl, C₂-C₇ alkoxyalkyl, C₂-C₄ cyanoalkyl, C₃-C₇ alkylthioalkyl or C₄-C₇ alkylcycloalkyl;
Embodiment P9. The compound of Embodiment P8 wherein
   R¹ is H, Me, halogen or cyclopropyl;
   R² is H or F;
   R³ is Me or F;
   R⁴ is H, CH₂OCOR¹⁴ or —S(O)₂R¹⁴;
   R⁵ is H;
   R⁶ is H, Me or OMe;
   R⁷ is H, Me or OMe;
   R⁸ is H, Me or OMe;
   G is OR¹⁰;
   R⁹ is H, Me or OMe;
   R¹⁰ is C₃-C₇ cycloalkyl, C₃-C₁₀ alkenylalkyl, C₃-C₁₀ alkynylalkyl, C₄-C₇ cycloalkylalkyl, C₂-C₇ alkoxyalkyl or C₄-C₇ alkylcycloalkyl.
Embodiment P10. The compound of Embodiment P9 wherein
   R¹ is H, Me, F, Cl, Br or cyclopropyl;
   R⁴ is H, CH₂OCO-t-Bu or SO₂CF₃;
   R⁸ is H;
   R⁹ is H; and
   R¹⁰ is C₃-C₇ cycloalkyl, C₃-C₁₀ alkenylalkyl or C₃-C₁₀ alkynylalkyl.
Embodiment P11. The compound of Embodiment P1 wherein
   Q is O;
   R¹ is H, C₁-C₇ alkyl, halogen, C₂-C₆ alkenyl, C₂-C₆ alkynyl, C₃-C₇ cycloalkyl, C₁-C₇ haloalkyl;
   R² is H, C₁-C₇ alkyl, halogen or CN;
   R³ is H, C₁-C₇ alkyl, halogen, CN, C₁-C₇ alkoxy or C₁-C₇ haloalkyl;
   R⁴ is H, C(═O)R¹⁴, —C(═S)R¹⁴, —CO₂R¹⁴, —C(═O)SR¹⁴, —S(O)₂R¹⁴, C(═O)NR¹³R¹⁴, —S(O)₂NR¹³R¹⁴, CH₂OC(═O)OR¹⁴, CH₂OC(═O)NR¹³R¹⁴ or CH₂OC(═O)R¹⁴;
   R⁵ is H, C₂-C₆ alkenyl, C₂-C₇ haloalkenyl, C₂-C₆ alkynyl, C₃-C₇ cycloalkyl, C₄-C₇ cycloalkylalkyl, C₃-C₇ alkenylalkyl, C₃-C₇ alkynylalkyl, C₂-C₇ alkoxyalkyl or C₄-C₇ alkylcycloalkyl;
   R⁶ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R⁷ is H, C₁-C₇ alkyl, C₂-C₆ alkenyl, C₂-C₆ alkynyl, C₃-C₇ cycloalkyl, C₃-C₇ alkenylalkyl, C₃-C₇ alkynylalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   R⁸ is H, C₁-C₇ alkyl, C₂-C₆ alkenyl, C₂-C₆ alkynyl, C₃-C₇ cycloalkyl, C₃-C₇ alkenylalkyl, C₃-C₇ alkynylalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
   G is OR¹⁰, SR¹⁰, SOR¹⁰ or SO₂R¹⁰;
   R¹⁰ is alkynylalkyl, C₄-C₁₀ alkylalkenylalkyl, C₄-C₁₀ alkylalkynylalkyl, C₄-C₇ alkylcycloalkyl, C₄-C₇ cycloalkylalkyl, C₄-C₇ halocycloalkylalkyl, C₄-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₂-C₇ alkoxyalkyl, C₂-C₄ cyanoalkyl or C₄-C₇ alkylcycloalkyl;
   R¹¹ is H or C₁-C₇ alkyl;
   R¹² is H or C₁-C₇ alkyl;
   R¹³ and R¹⁴ are independently H, C₁-C₇ haloalkyl or C₁-C₇ alkyl; and
   R^{f} is C₁-C₃ haloalkyl.
Embodiment P12. The compound of Embodiment P11 wherein
   R¹ is H, C₁-C₃ alkyl, halogen or C₃-C₄ cycloalkyl;
   R² is H, Me, F, Cl or CN;
   R³ is H, Me, F, Cl, —CN, OMe or CF₃;
   R⁴ is H, SO₂CF₃, SO₂CH₃, CO₂Me, COMe, CH₂OCO-t-Bu, CH₂OCO-n-Bu, CH₂OCO-c-hexyl, CH₂OCO-c-pentyl, CH₂OCOCH₂CH₃, COMe, CH₂OCOPh, CH₂OCO-i-Bu, CH₂OCOMe, CH₂OCO-sec-Bu, CH₂OCO-n-Pr and CH₂OCO-i-Pr or (C═O)SMe;
   R⁵ is H, C₄-C₇ cycloalkylalkyl or C₂-C₇ alkoxyalkyl;
   R⁶ is H, C₁-C₇ alkyl or C₁-C₇ alkoxy;
   R⁷ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
   R⁸ is H, C₁-C₇ alkyl or C₁-C₇ alkoxy;
   G is OR¹⁰ or SR¹⁰;
   R¹⁰ is C₃-C₇ cycloalkyl, C₃-C₇ halocycloalkyl, C₃-C₁₀ alkenylalkyl, C₃-C₁₀ alkynylalkyl, C₄-C₁₀ alkylalkenylalkyl, C₄-C₁₀ alkylalkynylalkyl, C₄-C₇ alkylcycloalkyl, C₄-C₇ cycloalkylalkyl, C₄-C₇ halocycloalkylalkyl, C₅-C₇ alkylcycloalkylalkyl, C₂-C₇ alkoxyalkyl, C₂-C₄ cyanoalkyl, C₃-C₇ alkylthioalkyl or C₄-C₇ alkylcycloalkyl.
Embodiment P13. The compound of Embodiment P12 wherein
   R¹ is H, Me, halogen or cyclopropyl;
   R² is H or F;
   R³ is Me or F;
   R⁴ is H, CH₂OCOR¹⁴ or —S(O)₂R¹⁴;
   R⁵ is H;
   R⁶ is H, Me or OMe;
   R⁷ is H, Me or OMe;
   R⁸ is H, Me or OMe;
   G is OR¹⁰;
   R¹⁰ is C₃-C₇ cycloalkyl, C₃-C₁₀ alkenylalkyl, C₃-C₁₀ alkynylalkyl, C₄-C₇ cycloalkylalkyl, C₂-C₇ alkoxyalkyl or C₄-C₇ alkylcycloalkyl.
Embodiment P14. The compound of Embodiment P13 wherein
   R¹ is H, Me, F, Cl, Br or cyclopropyl;
   R⁴ is H, CH₂OCO-t-Bu or SO₂CF₃;
   R⁸ is H; and
   R¹⁰ is C₃-C₇ cycloalkyl, C₃-C₁₀ alkenylalkyl or C₃-C₁₀ alkynylalkyl.
Embodiment P15. The compound of Embodiment P14 wherein
   R¹ is Me;
   R³ is Me;
   R⁴ is H;
   R⁶ is H;
   R⁷ is H; and
   R¹⁰ is cyclopropyl, cyclobutyl, cyclopentyl, allyl or propargyl.
Specific embodiments include compounds of Formula 1 selected from the group consisting of:

N-[5-[3-(cyclopentyloxy)-2-oxo-1-pyrrolidinyl]-2,4-dimethylphenyl]-
1,1,1-trifluoromethanesulfonamide (Compound 6);
[[5-[3-(cyclopentyloxy)-2-oxo-1-pyrrolidinyl]-2,4-
dimethylphenyl][(trifluoromethyl)sulfonyl]amino]methyl
2,2-dimethylpropanoate (Compound 5)
N-[2,4-dimethyl-5-[2-oxo-3-(2-propyn-1-yloxy)-1-pyrrolidinyl]phenyl]-
1,1,1-trifluoromethanesulfonamide (Compound 1);
N-[5-[3-(cyclopropyloxy)-2-oxo-1-pyrrolidinyl]-2,4-dimethylphenyl]-
1,1,1-trifluoromethanesulfonamide (Compound 3);
[[5-[3-(cyclopropyloxy)-2-oxo-1-pyrrolidinyl]-2,4-
dimethylphenyl][(trifluoromethyl)sulfonyl]amino]methyl
2,2-dimethylpropanoate (Compound 7);
[[5-[3-(cyclobutyloxy)-2-oxo-1-pyrrolidinyl]-2,4-
dimethylphenyl][(trifluoromethyl)sulfonyl]amino]methyl
2,2-dimethylpropanoate (Compound 8);
N-[2,4-dimethyl-5-[2-oxo-3-(2-propen-1-yloxy)-1-pyrrolidinyl]phenyl]-
1,1,1-trifluoromethanesulfonamide (Compound 2); and
N-[5-[3-(cyclobutyloxy)-2-oxo-1-pyrrolidinyl]-2,4-dimethylphenyl]-
1,1,1-trifluoromethanesulfonamide (Compound 4).
N-[5-[3-(ethoxyimino)-2-oxo-1-pyrrolidinyl]-2,4-dimethylphenyl]-1,1,1-
trifluoromethanesulfonamide (Compound 12)
N-[2,4-dimethyl-5-[2-oxo-3-[(2-propyn-1-yloxy)imino]-1-
pyrrolidinyl]phenyl]-1,1,1-trifluoromethanesulfonamide (Compound 13)
1,1,1-trifluoro-N-[5-[3-(methoxyimino)-2-oxo-1-pyrrolidinyl]-2,4-
dimethylphenyl]methanesulfonamide (Compound 9)

This invention also relates to a method for controlling undesired vegetation comprising applying to the locus of the vegetation herbicidally effective amounts of the compounds of the invention (e.g., as a composition described herein). Of note as embodiments relating to methods of use are those involving the compounds of embodiments described above. Compounds of the invention are particularly useful for selective control of weeds in crops such as wheat, barley, maize, soybean, sunflower, cotton, oilseed rape and rice, and specialty crops such as sugarcane, citrus, fruit and nut crops.

Also noteworthy as embodiments are herbicidal compositions of the present invention comprising the compounds of embodiments described above.

This invention also includes a herbicidal mixture comprising (a) a compound selected from Formula 1, N-oxides, and salts thereof, and (b) at least one additional active ingredient selected from (b1) photosystem II inhibitors, (b2) acetohydroxy acid synthase (AHAS) inhibitors, (b3) acetyl-CoA carboxylase (ACCase) inhibitors, (b4) auxin mimics, (b5) 5-enol-pyruvylshikimate-3-phosphate (EPSP) synthase inhibitors, (b6) photosystem I electron diverters, (b7) protoporphyrinogen oxidase (PPO) inhibitors, (b8) glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solanesyltransferase (HST) inhibitors, (b14) cellulose biosynthesis inhibitors, (b15) other herbicides including mitotic disruptors organic arsenicals, asulam, bromobutide, cinmethylin, cumyluron, dazomet, difenzoquat, dymron, etobenzanid, flurenol, fosamine, fosamine-ammonium, hydantocidin, metam, methyldymron, oleic acid, oxaziclomefone, pelargonic acid and pyributicarb, (b16) herbicide safeners, and salts of compounds of (b1) through (b16).

“Photosystem II inhibitors” (b1) are chemical compounds that bind to the D-1 protein at the Q_{B}-binding niche and thus block electron transport from Q_{A} to Q_{B} in the chloroplast thylakoid membranes. The electrons blocked from passing through photosystem II are transferred through a series of reactions to form toxic compounds that disrupt cell membranes and cause chloroplast swelling, membrane leakage, and ultimately cellular destruction. The Q_{B}-binding niche has three different binding sites: binding site A binds the triazines such as atrazine, triazinones such as hexazinone, and uracils such as bromacil, binding site B binds the phenylureas such as diuron, and binding site C binds benzothiadiazoles such as bentazon, nitriles such as bromoxynil and phenyl-pyridazines such as pyridate. Examples of photosystem II inhibitors include ametryn, amicarbazone, atrazine, bentazon, bromacil, bromofenoxim, bromoxynil, chlorbromuron, chloridazon, chlorotoluron, chloroxuron, cumyluron, cyanazine, daimuron, desmedipham, desmetryn, dimefuron, dimethametryn, diuron, ethidimuron, fenuron, fluometuron, hexazinone, ioxynil, isoproturon, isouron, lenacil, linuron, metamitron, methabenzthiazuron, metobromuron, metoxuron, metribuzin, monolinuron, neburon, pentanochlor, phenmedipham, prometon, prometryn, propanil, propazine, pyridafol, pyridate, siduron, simazine, simetryn, tebuthiuron, terbacil, terbumeton, terbuthylazine, terbutryn and trietazine.

“AHAS inhibitors” (b2) are chemical compounds that inhibit acetohydroxy acid synthase (AHAS), also known as acetolactate synthase (ALS), and thus kill plants by inhibiting the production of the branched-chain aliphatic amino acids such as valine, leucine and isoleucine, which are required for protein synthesis and cell growth. Examples of AHAS inhibitors include amidosulfuron, azimsulfuron, bensulfuron-methyl, bispyribac-sodium, cloransulam-methyl, chlorimuron-ethyl, chlorsulfuron, cinosulfuron, cyclosulfamuron, diclosulam, ethametsulfuron-methyl, ethoxysulfuron, flazasulfuron, florasulam, flucarbazone-sodium, flumetsulam, flupyrsulfuron-methyl, flupyrsulfuron-sodium, foramsulfuron, halosulfuron-methyl, imazamethabenz-methyl, imazamox, imazapic, imazapyr, imazaquin, imazethapyr, imazosulfuron, iodosulfuron-methyl (including sodium salt), iofensulfuron (2-iodo-N-[[(4-methoxy-6-methyl-1,3,5-triazin-2-yl)amino]carbonyl]benzenesulfonamide), mesosulfuron-methyl, metazosulfuron (3-chloro-4-(5,6-dihydro-5-methyl-1,4,2-dioxazin-3-yl)-N-[[(4,6-dimethoxy-2-pyrimidinyl)amino]carbonyl]-1-methyl-1H-pyrazole-5-sulfonamide), metosulam, metsulfuron-methyl, nicosulfuron, oxasulfuron, penoxsulam, primisulfuron-methyl, propoxycarbazone-sodium, propyrisulfuron (2-chloro-N-[[(4,6-dimethoxy-2-pyrimidinyl)amino]carbonyl]-6-propylimidazo[1,2-b]pyridazine-3-sulfonamide), prosulfuron, pyrazosulfuron-ethyl, pyribenzoxim, pyriftalid, pyriminobac-methyl, pyrithiobac-sodium, rimsulfuron, sulfometuron-methyl, sulfosulfuron, thiencarbazone, thifensulfuron-methyl, triafamone (N-[2-[(4,6-dimethoxy-1,3,5-triazin-2-yl)carbonyl]-6-fluorophenyl]-1,1-difluoro-N-methylmethanesulfonamide), triasulfuron, tribenuron-methyl, trifloxysulfuron (including sodium salt), triflusulfuron-methyl and tritosulfuron.

“ACCase inhibitors” (b3) are chemical compounds that inhibit the acetyl-CoA carboxylase enzyme, which is responsible for catalyzing an early step in lipid and fatty acid synthesis in plants. Lipids are essential components of cell membranes, and without them, new cells cannot be produced. The inhibition of acetyl CoA carboxylase and the subsequent lack of lipid production leads to losses in cell membrane integrity, especially in regions of active growth such as meristems. Eventually shoot and rhizome growth ceases, and shoot meristems and rhizome buds begin to die back. Examples of ACCase inhibitors include alloxydim, butroxydim, clethodim, clodinafop, cycloxydim, cyhalofop, diclofop, fenoxaprop, fluazifop, haloxyfop, pinoxaden, profoxydim, propaquizafop, quizalofop, sethoxydim, tepraloxydim and tralkoxydim, including resolved forms such as fenoxaprop-P, fluazifop-P, haloxyfop-P and quizalofop-P and ester forms such as clodinafop-propargyl, cyhalofop-butyl, diclofop-methyl and fenoxaprop-P-ethyl.

Auxin is a plant hormone that regulates growth in many plant tissues. “Auxin mimics” (b4) are chemical compounds mimicking the plant growth hormone auxin, thus causing uncontrolled and disorganized growth leading to plant death in susceptible species. Examples of auxin mimics include aminocyclopyrachlor (6-amino-5-chloro-2-cyclopropyl-4-pyrimidinecarboxylic acid) and its methyl and ethyl esters and its sodium and potassium salts, aminopyralid, benazolin-ethyl, chloramben, clacyfos, clomeprop, clopyralid, dicamba, 2,4-D, 2,4-DB, dichlorprop, fluroxypyr, halauxifen (4-amino-3-chloro-6-(4-chloro-2-fluoro-3-methoxyphenyl)-2-pyridinecarboxylic acid), halauxifen-methyl (methyl 4-amino-3-chloro-6-(4-chloro-2-fluoro-3-methoxyphenyl)-2-pyridinecarboxylate), MCPA, MCPB, mecoprop, picloram, quinclorac, quinmerac, 2,3,6-TBA, triclopyr, and methyl 4-amino-3-chloro-6-(4-chloro-2-fluoro-3-methoxyphenyl)-5-fluoro-2-pyridinecarboxylate.

“EPSP synthase inhibitors” (b5) are chemical compounds that inhibit the enzyme, 5-enol-pyruvylshikimate-3-phosphate synthase, which is involved in the synthesis of aromatic amino acids such as tyrosine, tryptophan and phenylalanine. EPSP inhibitor herbicides are readily absorbed through plant foliage and translocated in the phloem to the growing points. Glyphosate is a relatively nonselective postemergence herbicide that belongs to this group. Glyphosate includes esters and salts such as ammonium, isopropylammonium, potassium, sodium (including sesquisodium) and trimesium (alternatively named sulfosate).

“Photosystem I electron diverters” (b6) are chemical compounds that accept electrons from Photosystem I, and after several cycles, generate hydroxyl radicals. These radicals are extremely reactive and readily destroy unsaturated lipids, including membrane fatty acids and chlorophyll. This destroys cell membrane integrity, so that cells and organelles “leak”, leading to rapid leaf wilting and desiccation, and eventually to plant death. Examples of this second type of photosynthesis inhibitor include diquat and paraquat.

“PPO inhibitors” (b7) are chemical compounds that inhibit the enzyme protoporphyrinogen oxidase, quickly resulting in formation of highly reactive compounds in plants that rupture cell membranes, causing cell fluids to leak out. Examples of PPO inhibitors include acifluorfen-sodium, azafenidin, benzfendizone, bifenox, butafenacil, carfentrazone, carfentrazone-ethyl, chlomethoxyfen, cinidon-ethyl, fluazolate, flufenpyr-ethyl, flumiclorac-pentyl, flumioxazin, fluoroglycofen-ethyl, fluthiacet-methyl, fomesafen, halosafen, lactofen, oxadiargyl, oxadiazon, oxyfluorfen, pentoxazone, profluazol, pyraclonil, pyraflufen-ethyl, saflufenacil, sulfentrazone, thidiazimin, trifludimoxazin (dihydro-1,5-dimehyl-6-thioxo-3-[2,2,7-trifluoro-3,4-dihydro-3-oxo-4-(2-propyn-1-yl)-2H-1,4-benzoxazin-6-yl]-1,3,5-triazine-2,4(1H,3H)-dione) and tiafenacil (methyl N-[2-[[2-chloro-5-[3,6-dihydro-3-methyl-2,6-dioxo-4-(trifluoromethyl)-1(2H)-pyrimidinyl]-4-fluorophenyl]thio]-1-oxopropyl]-β-alaninate).

“GS inhibitors” (b8) are chemical compounds that inhibit the activity of the glutamine synthetase enzyme, which plants use to convert ammonia into glutamine. Consequently, ammonia accumulates and glutamine levels decrease. Plant damage probably occurs due to the combined effects of ammonia toxicity and deficiency of amino acids required for other metabolic processes. The GS inhibitors include glufosinate and its esters and salts such as glufosinate-ammonium and other phosphinothricin derivatives, glufosinate-P ((2S)-2-amino-4-(hydroxymethylphosphinyl)butanoic acid) and bilanaphos.

“VLCFA elongase inhibitors” (b9) are herbicides having a wide variety of chemical structures, which inhibit the elongase. Elongase is one of the enzymes located in or near chloroplasts which are involved in biosynthesis of VLCFAs. In plants, very-long-chain fatty acids are the main constituents of hydrophobic polymers that prevent desiccation at the leaf surface and provide stability to pollen grains. Such herbicides include acetochlor, alachlor, anilofos, butachlor, cafenstrole, dimethachlor, dimethenamid, diphenamid, fenoxasulfone (3-[[(2,5-dichloro-4-ethoxyphenyl)methyl]sulfonyl]-4,5-dihydro-5,5-dimethylisoxazole), fentrazamide, flufenacet, indanofan, mefenacet, metazachlor, metolachlor, naproanilide, napropamide, napropamide-M ((2R)—N,N-diethyl-2-(1-naphthalenyloxy)propanamide), pethoxamid, piperophos, pretilachlor, propachlor, propisochlor, pyroxasulfone, and thenylchlor, including resolved forms such as S-metolachlor and chloroacetamides and oxyacetamides.

“Auxin transport inhibitors” (b10) are chemical substances that inhibit auxin transport in plants, such as by binding with an auxin-carrier protein. Examples of auxin transport inhibitors include diflufenzopyr, naptalam (also known as N-(1-naphthyl)phthalamic acid and 2-[(1-naphthalenylamino)carbonyl]benzoic acid).

“PDS inhibitors” (b11) are chemical compounds that inhibit carotenoid biosynthesis pathway at the phytoene desaturase step. Examples of PDS inhibitors include beflubutamid, diflufenican, fluridone, flurochloridone, flurtamone norflurzon and picolinafen.

“HPPD inhibitors” (b12) are chemical substances that inhibit the biosynthesis of synthesis of 4-hydroxyphenyl-pyruvate dioxygenase. Examples of HPPD inhibitors include benzobicyclon, benzofenap, bicyclopyrone (4-hydroxy-3-[[2-[(2-methoxyethoxy)methyl]-6-(trifluoromethyl)-3-pyridinyl]carbonyl]bicyclo[3.2.1]oct-3-en-2-one), fenquinotrione (2-[[8-chloro-3,4-dihydro-4-(4-methoxyphenyl)-3-oxo-2-quinoxalinyl]carbonyl]-1,3-cyclohexanedione), isoxachlortole, isoxaflutole, mesotrione, pyrasulfotole, pyrazolynate, pyrazoxyfen, sulcotrione, tefuryltrione, tembotrione, tolpyralate (1-[[1-ethyl-4-[3-(2-methoxyethoxy)-2-methyl-4-(methylsulfonyl)benzoyl]-1H-pyrazol-5-yl]oxy]ethyl methyl carbonate), topramezone, 5-chloro-3-[(2-hydroxy-6-oxo-1-cyclohexen-1-yl)carbonyl]-1-(4-methoxyphenyl)-2(1H)-quinoxalinone, 4-(2,6-diethyl-4-methylphenyl)-5-hydroxy-2,6-dimethyl-3(2H)-pyridazinone, 4-(4-fluorophenyl)-6-[(2-hydroxy-6-oxo-1-cyclohexen-1-yl)carbonyl]-2-methyl-1,2,4-triazine-3,5(2H,4H)-dione, 5-[(2-hydroxy-6-oxo-1-cyclohexen-1-yl)carbonyl]-2-(3-methoxyphenyl)-3-(3-methoxypropyl)-4(3H)-pyrimidinone, 2-methyl-N-(4-methyl-1,2,5-oxadiazol-3-yl)-3-(methylsulfinyl)-4-(trifluoromethyl)benzamide and 2-methyl-3-(methylsulfonyl)-N-(1-methyl-1H-tetrazol-5-yl)-4-(trifluoromethyl)benzamide.

“HST inhibitors” (b13) disrupt a plant's ability to convert homogentisate to 2-methyl-6-solanyl-1,4-benzoquinone, thereby disrupting carotenoid biosynthesis. Examples of HST inhibitors include haloxydine, pyriclor, 3-(2-chloro-3,6-difluorophenyl)-4-hydroxy-1-methyl-1,5-naphthyridin-2(1H)-one, 7-(3,5-dichloro-4-pyridinyl)-5-(2,2-difluoroethyl)-8-hydroxypyrido[2,3-b]pyrazin-6(5H)-one and 4-(2,6-diethyl-4-methylphenyl)-5-hydroxy-2,6-dimethyl-3(2H)-pyridazinone.

HST inhibitors also include compounds of Formulae A and B.

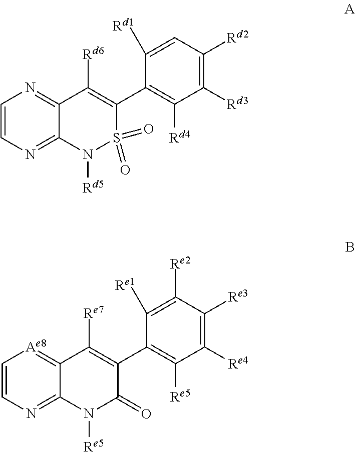

wherein R^{d1} is H, Cl or CF₃; R^{d2} is H, Cl or Br; R^{d3} is H or Cl; R^{d4} is H, Cl or CF₃; R^{d5} is CH₃, CH₂CH₃ or CH₂CHF₂; and R^{d6} is OH or —OC(═O)-i-Pr; and R^{e1} is H, F, Cl, CH₃ or CH₂CH₃; R^{e2} is H or CF₃; R^{e3} is H, CH₃ or CH₂CH₃; R^{e4} is H, F or Br; R^{e5} is Cl, CH₃, CF₃, OCF₃ or CH₂CH₃; R^{e6} is H, CH₃, CH₂CHF₂ or C═CH; R^{e7} is OH, —OC(═O)Et, —OC(═O)-i-Pr or —OC(═O)-t-Bu; and A^{e8} is N or CH.

“Cellulose biosynthesis inhibitors” (b14) inhibit the biosynthesis of cellulose in certain plants. They are most effective when applied preemergence or early postemergence on young or rapidly growing plants. Examples of cellulose biosynthesis inhibitors include chlorthiamid, dichlobenil, flupoxam, indaziflam (N²-[(1R,2S)-2,3-dihydro-2,6-dimethyl-1H-inden-1-yl]-6-(1-fluoroethyl)-1,3,5-triazine-2,4-diamine), isoxaben and triaziflam.

“Other herbicides” (b15) include herbicides that act through a variety of different modes of action such as mitotic disruptors (e.g., flamprop-M-methyl and flamprop-M-isopropyl) organic arsenicals (e.g., DSMA, and MSMA), 7,8-dihydropteroate synthase inhibitors, chloroplast isoprenoid synthesis inhibitors and cell-wall biosynthesis inhibitors. Other herbicides include those herbicides having unknown modes of action or do not fall into a specific category listed in (b1) through (b14) or act through a combination of modes of action listed above. Examples of other herbicides include aclonifen, asulam, amitrole, bromobutide, cinmethylin, clomazone, cumyluron, cyclopyrimorate (6-chloro-3-(2-cyclopropyl-6-methylphenoxy)-4-pyridazinyl 4-morpholinecarboxylate), daimuron, difenzoquat, etobenzanid, fluometuron, flurenol, fosamine, fosamine-ammonium, dazomet, dymron, ipfencarbazone (1-(2,4-dichlorophenyl)-N-(2,4-difluorophenyl)-1,5-dihydro-N-(1-methylethyl)-5-oxo-4H-1,2,4-triazole-4-carboxamide), metam, methyldymron, oleic acid, oxaziclomefone, pelargonic acid, pyributicarb and 5-[[(2,6-difluorophenyl)methoxy]methyl]-4,5-dihydro-5-methyl-3-(3-methyl-2-thienyl)isoxazole.

“Other herbicides” (b15) also include a compound of Formula (b15A)

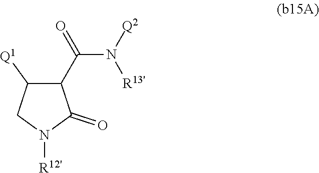

wherein
R^{12′} is H, C₁-C₆ alkyl, C₁-C₆ haloalkyl or C₄-C₈ cycloalkyl;
R^{13′} is H, C₁-C₆ alkyl or C₁-C₆ alkoxy;
Q¹ is an optionally substituted ring system selected from the group consisting of phenyl, thienyl, pyridinyl, benzodioxolyl, naphthalenyl, benzofuranyl, furanyl, benzothiophenyl and pyrazolyl, wherein when substituted said ring system is substituted with 1 to 3 R^{14′};
Q² is and optionally substituted ring system selected from the group consisting of phenyl, pyridinyl, benzodioxolyl, pyridinonyl, thiadiazolyl, thiazolyl, and oxazolyl, wherein when substituted said ring system is substituted with 1 to 3 R¹⁵′;
each R^{14′} is independently halogen, C₁-C₆ alkyl, C₁-C₆ haloalkyl, C₁-C₆ alkoxy, C₁-C₆ haloalkoxy, C₃-C₈ cyaloalkyl, cyano, C₁-C₆ alkylthio, C₁-C₆ alkylsulfinyl, C₁-C₆ alkylsulfonyl, SF₅, NHR¹⁷; or phenyl optionally substituted by 1 to 3 R¹⁶; or pyrazolyl optionally substituted by 1 to 3 R¹⁶;
each R^{15′} is independently halogen, C₁-C₆ alkyl, C₁-C₆ haloalkyl, C₁-C₆ alkoxy, C₁-C₆ haloalkoxy, cyano, nitro, C₁-C₆ alkylthio, C₁-C₆ alkylsulfinyl, C₁-C₆ alkylsulfonyl;
each R^{16′} is independently halogen, C₁-C₆ alkyl or C₁-C₆ haloalkyl; and
R^{17′} is C₁-C₄ alkoxycarbonyl.

In one Embodiment wherein “other herbicides” (b15) also include a compound of Formula (b15A), it is preferred that R^{12′} is H or C₁-C₆ alkyl; more preferably R^{12′} is H or methyl. Preferrably R^{13′} is H. Preferably Qi is either a phenyl ring or a pyridinyl ring, each ring substituted by 1 to 3 R^{14′}; more preferably Q¹ is a phenyl ring substituted by 1 to 2 R^{14′}.

Preferably Q² is a phenyl ring substituted with 1 to 3 R^{15′}; more preferably Q² is a phenyl ring substituted by 1 to 2 R^{15′}. Preferably each R^{14′} is independently halogen, C₁-C₄ alkyl, C₁-C₃ haloalkyl, C₁-C₃ alkoxy or C₁-C₃ haloalkoxy; more preferably each R^{14′} is independently chloro, fluoro, bromo, C₁-C₂ haloalkyl, C₁-C₂ haloalkoxy or C₁-C₂ alkoxy. Preferrably each R^{15′} is independently halogen, C₁-C₄ alkyl, C₁-C₃ haloalkoxy; more preferably each R^{15′} is independently chloro, fluoro, bromo, C₁-C₂ haloalkyl, C₁-C₂ haloalkoxy or C₁-C₂ alkoxy.

Specifically preferred as “other herbicides” (b15) include any one of the following (b15A-1) through (b15A-15):

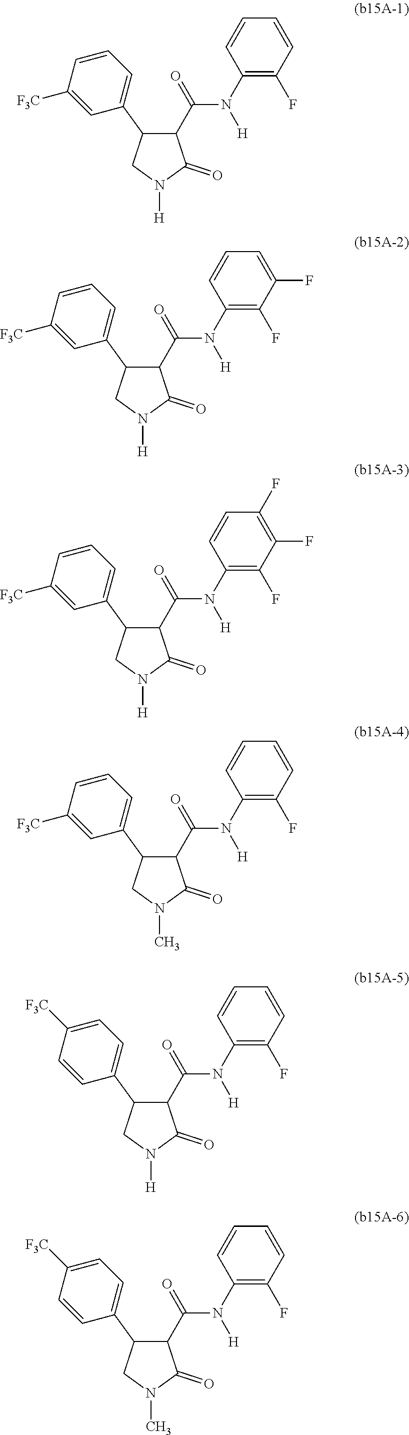 [SMILES: O=C1NCC(c2cccc(C(F)(F)F)c2)C1C(=O)Nc1ccccc1F ; O=C1NCC(c2cccc(C(F)(F)F)c2)C1C(=O)Nc1cccc(F)c1F ; O=C1NCC(c2cccc(C(F)(F)F)c2)C1C(=O)Nc1ccc(F)c(F)c1F ; CN1CC(c2cccc(C(F)(F)F)c2)C(C(=O)Nc2ccccc2F)C1=O ; O=C1NCC(c2ccc(C(F)(F)F)cc2)C1C(=O)Nc1ccccc1F ; CN1CC(c2ccc(C(F)(F)F)cc2)C(C(=O)Nc2ccccc2F)C1=O]

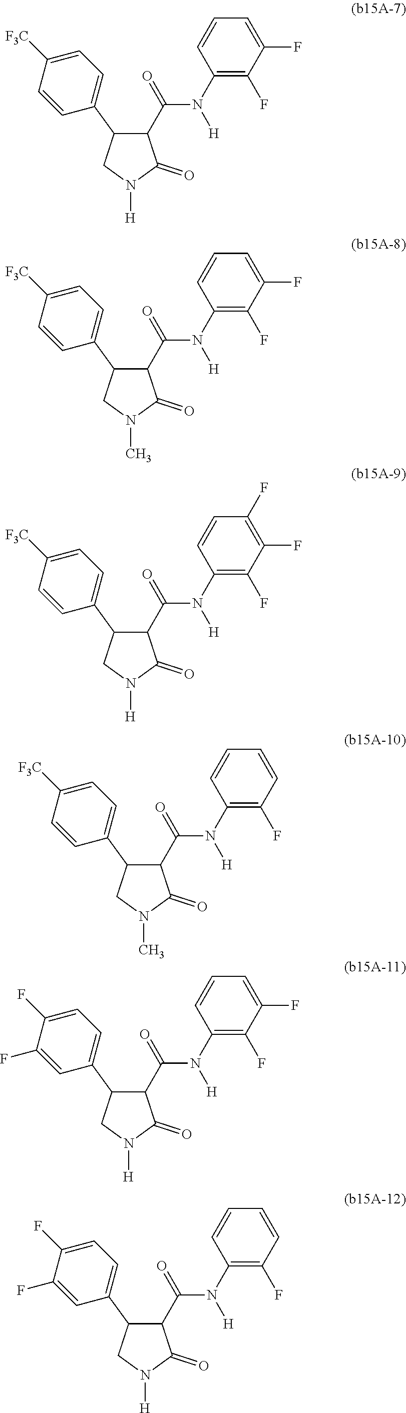 [SMILES: O=C1NCC(c2ccc(C(F)(F)F)cc2)C1C(=O)Nc1cccc(F)c1F ; CN1CC(c2ccc(C(F)(F)F)cc2)C(C(=O)Nc2cccc(F)c2F)C1=O ; O=C1NCC(c2ccc(C(F)(F)F)cc2)C1C(=O)Nc1ccc(F)c(F)c1F ; CN1CC(c2ccc(C(F)(F)F)cc2)C(C(=O)Nc2ccccc2F)C1=O ; O=C1NCC(c2ccc(F)c(F)c2)C1C(=O)Nc1cccc(F)c1F ; O=C1NCC(c2ccc(F)c(F)c2)C1C(=O)Nc1ccccc1F]

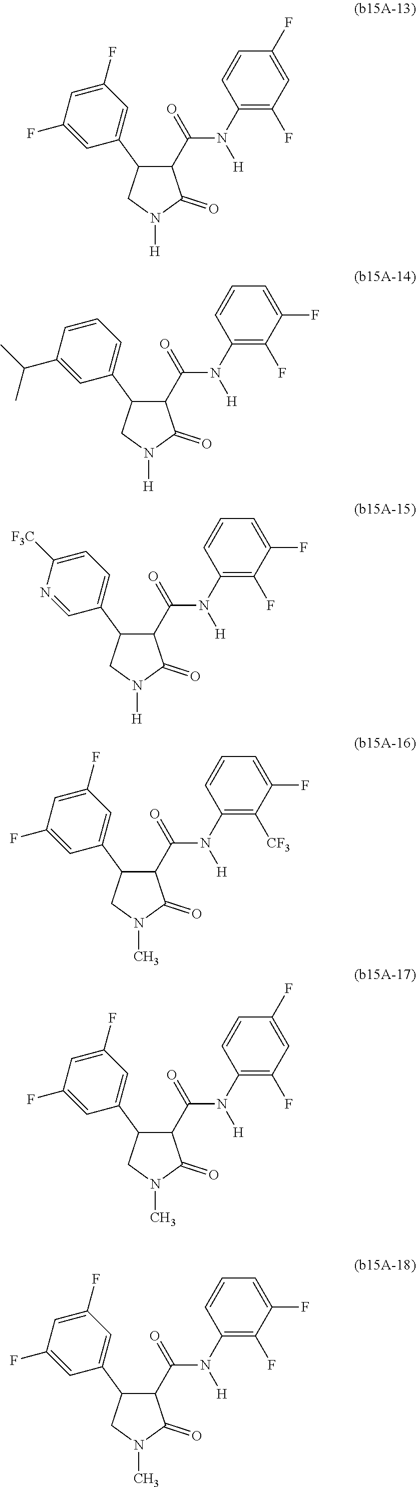 [SMILES: O=C1NCC(c2cc(F)cc(F)c2)C1C(=O)Nc1ccc(F)cc1F ; CC(C)c1cccc(C2CNC(=O)C2C(=O)Nc2cccc(F)c2F)c1 ; O=C1NCC(c2ccc(C(F)(F)F)nc2)C1C(=O)Nc1cccc(F)c1F ; CN1CC(c2cc(F)cc(F)c2)C(C(=O)Nc2cccc(F)c2C(F)(F)F)C1=O ; CN1CC(c2cc(F)cc(F)c2)C(C(=O)Nc2ccc(F)cc2F)C1=O ; CN1CC(c2cc(F)cc(F)c2)C(C(=O)Nc2cccc(F)c2F)C1=O]

“Other herbicides” (b15) also include a compound of Formula (b15B)

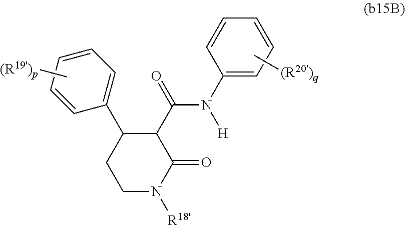

wherein
R^{18′} is H, C₁-C₆ alkyl, C₁-C₆ haloalkyl or C₄-C₈ cycloalkyl;
each R^{19′} is independently halogen, C₁-C₆ haloalkyl or C₁-C₆ haloalkoxy;
p is an integer of 0, 1, 2 or 3;
each R^{20′} is independently halogen, C₁-C₆ haloalkyl or C₁-C₆ haloalkoxy; and
q is an integer of 0, 1, 2 or 3.

In one Embodiment wherein “other herbicides” (b15) also include a compound of Formula (b15B), it is preferred that R¹⁸ is H, methyl, ethyl or propyl; more preferably R¹⁸ is H or methyl; most preferably R¹⁸ is H. Preferrably each R¹⁹ is independently chloro, fluoro, C₁-C₃ haloalkyl or C₁-C₃ haloalkoxy; more preferably each R¹⁹ is independently chloro, fluoro, C₁ fluoroalkyl (i.e. fluoromethyl, difluoromethyl or trifluoromethyl) or C₁ fluoroalkoxy (i.e. trifluoromethoxy, difluoromethoxy or fluoromethoxy). Preferably each R²⁰ is independently chloro, fluoro, C₁ haloalkyl or C₁ haloalkoxy; more preferably each R²⁰ is independently chloro, fluoro, C₁ fluoroalkyl (i.e. fluoromethyl, difluoromethyl or trifluromethyl) or C₁ fluoroalkoxy (i.e. trifluoromethoxy, difluoromethoxy or fluoromethoxy).

Specifically preferred as “other herbicides” (b15) include any one of the following (b15B-1) through (b15B-19):

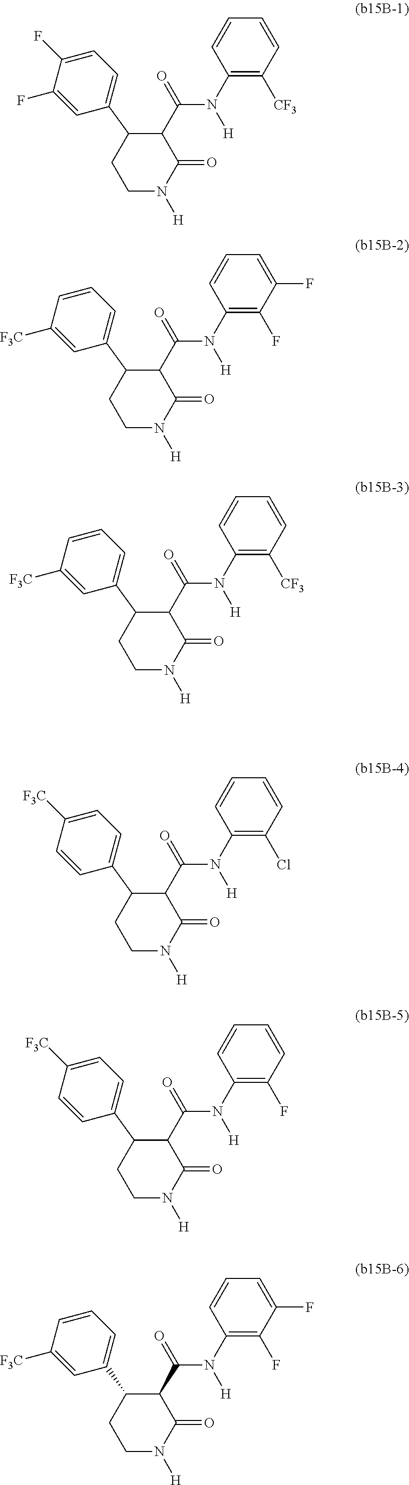 [SMILES: O=C1NCCC(c2ccc(F)c(F)c2)C1C(=O)Nc1ccccc1C(F)(F)F ; O=C1NCCC(c2cccc(C(F)(F)F)c2)C1C(=O)Nc1cccc(F)c1F ; O=C1NCCC(c2cccc(C(F)(F)F)c2)C1C(=O)Nc1ccccc1C(F)(F)F ; O=C1NCCC(c2ccc(C(F)(F)F)cc2)C1C(=O)Nc1ccccc1Cl ; O=C1NCCC(c2ccc(C(F)(F)F)cc2)C1C(=O)Nc1ccccc1F ; O=C1NCC[C@H](c2cccc(C(F)(F)F)c2)[C@H]1C(=O)Nc1cccc(F)c1F]

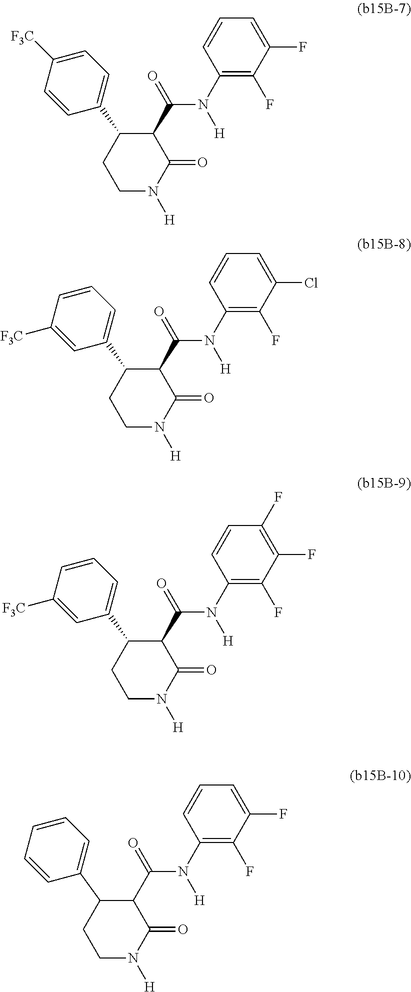 [SMILES: O=C1NCC[C@H](c2ccc(C(F)(F)F)cc2)[C@H]1C(=O)Nc1cccc(F)c1F ; O=C1NCC[C@H](c2cccc(C(F)(F)F)c2)[C@H]1C(=O)Nc1cccc(Cl)c1F ; O=C1NCC[C@H](c2cccc(C(F)(F)F)c2)[C@H]1C(=O)Nc1ccc(F)c(F)c1F ; O=C1NCCC(c2ccccc2)C1C(=O)Nc1cccc(F)c1F]

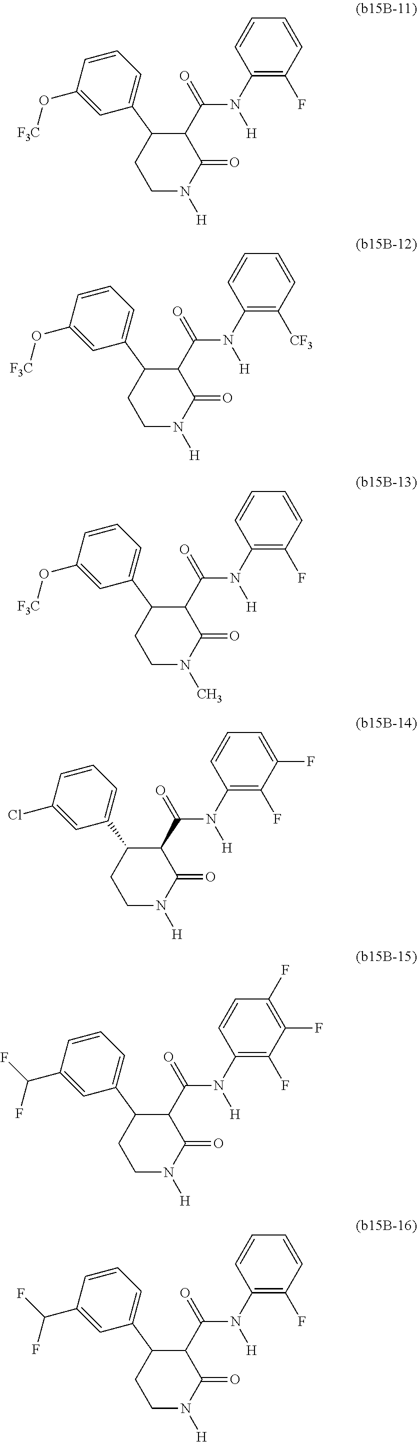

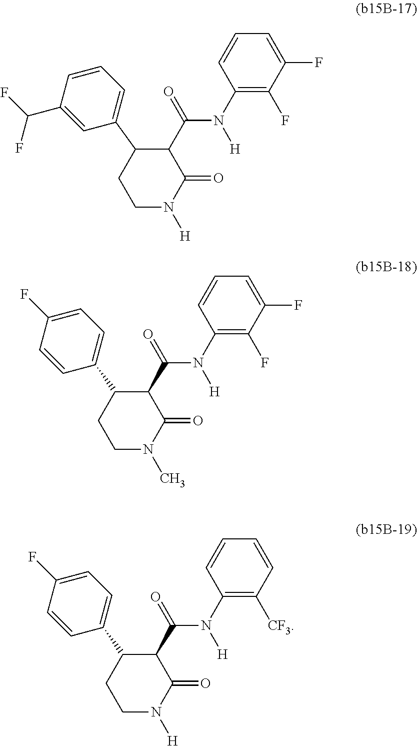

Another Embodiment wherein “other herbicides” (b15) also include a compound of Formula (b15C),

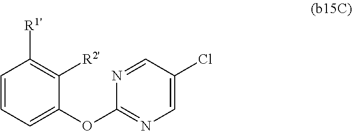

wherein R^{1′} is Cl, Br or CN; and R^{2′} is C(═O)CH₂CH₂CF₃, CH₂CH₂CH₂CH₂CF₃ or 3-CHF₂-isoxazol-5-yl.

“Herbicide safeners” (b16) are substances added to a herbicide formulation to eliminate or reduce phytotoxic effects of the herbicide to certain crops. These compounds protect crops from injury by herbicides but typically do not prevent the herbicide from controlling undesired vegetation. Examples of herbicide safeners include but are not limited to benoxacor, cloquintocet-mexyl, cumyluron, cyometrinil, cyprosulfamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone, naphthalic anhydride, oxabetrinil, N-(aminocarbonyl)-2-methylbenzenesulfonamide and N-(aminocarbonyl)-2-fluorobenzenesulfonamide, 1-bromo-4-[(chloromethyl)sulfonyl]benzene, 2-(dichloromethyl)-2-methyl-1,3-dioxolane (MG 191), 4-(dichloroacetyl)-1-oxa-4-azospiro[4.5]decane (MON 4660), 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide.

One or more of the following methods and variations as described in Schemes 1-13 can be used to prepare the compounds of Formula 1. The definitions of G, Q, X, R¹-R¹⁰, and R^{f} in the compounds of Formulae 1-19 below are as defined above in the Summary of the Disclosure unless otherwise noted. Compounds of Formulae 1a, 1b, 1c, 1d, 3a, 4a, 4b, 4c, 5a and 5b are various subsets of the compounds of Formulae 1, 3, 4 and 5; and all substituents for Formulae 1a, 1b, 1c, 1d, 3a, 4a, 4b, 4c, 5a and 5b are as defined above for Formula 1 unless otherwise noted in the disclosure including the schemes.

As outlined in Scheme 1, compounds of Formula 1a (i.e a compound of Formula 1, wherein R⁴ is H) can be made by reaction of an appropriately substituted aniline of Formula 2 with 1 equivalent (or a slightly excess over 1 equivalent) of a haloalkylsulfonyl chloride of Formula R^{f}SO₂Cl or a corresponding haloalkylsulfonyl anhydride of Formula R^{f}(SO₂)₂O in the presence of a suitable base, in a compatible solvent including but not limited to tetrahydrofuran, acetonitrile, toluene, diethyl ether, dioxane, dichloromethane or N,N-dimethylformamide, at temperatures generally ranging from 0° C. to ambient temperature. Some examples of the suitable base can be pyridine, triethylamine, Hunig's base or potassium carbonate. Alternatively, bis-sulfonamides of Formula 1b (i.e a compound of Formula 1, wherein R⁴ is SO₂R^{f} and R^{f} is haloalkyl) are accessible by reacting an aniline of Formula 2 with 2 equivalents (or an excess over 2.0 equivalents) of a haloalkylsulfonyl chloride of Formula R^{f}SC₂Cl or a corresponding haloalkylsulfonyl anhydride of Formula R^{f}(SO₂)₂O under similar reaction conditions described as above. Treating bis-sulfonamides of Formula 1b with an excess of aqueous base followed by neutralization or acidification with acid readily provides the corresponding mono-sulfonamide of Formula 1a. Preferred conditions for this hydrolysis are usually aqueous sodium or potassium hydroxide, optionally used with a cosolvent such as methanol, ethanol, dioxane or tetrahydrofuran, followed by neutralization or acidification with concentrated or aqueous hydrochloric acid.

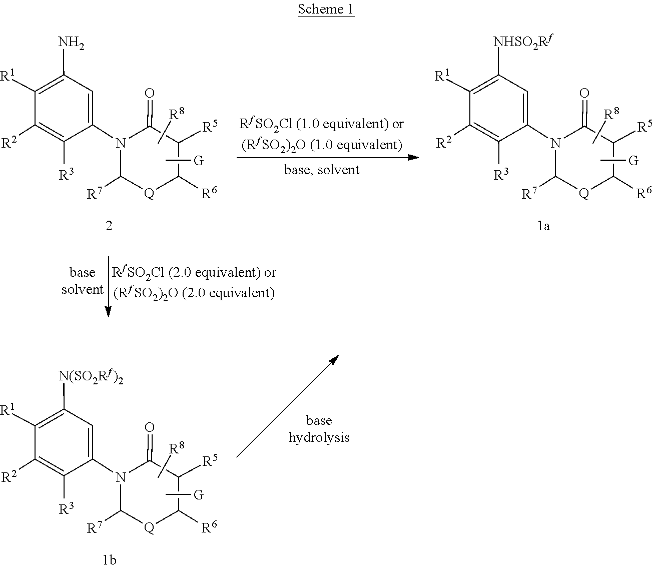

Substituted anilines of Formula 2 are readily accessed by hydrogenation of nitrobenzenes of Formula 3 under conditions that include but not limited to catalytic hydrogenation with 5-10% palladium metal on carbon or platinum oxide in solvents such as methanol, ethanol or ethyl acetate under an atmosphere of hydrogen. This reaction can generally be done in a Parr Hydrogenator. Alternatively, reduction of the nitro group can be accomplished with activated zinc metal in acetic acid, with stannous chloride in aqueous hydrochloric acid, iron metal in acetic acid or in aqueous alcohol or in an aqueous ethyl acetate mixture with ammonium chloride (i.e. Fe with 3 equivalents of ammonium chloride in aqueous ethanol) or with sodium borohydride in methanol in the presence of NiAC₂-4H₂O (see J. Am. Chem. Soc., 2005, 119).

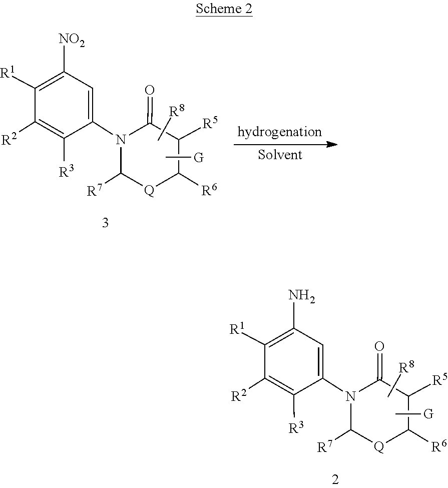

Intermediates of Formula 3 can be accessed by copper-mediated coupling of a meta-bromo or meta-iodo substituted nitrobenzene of Formula 4a or 4b (wherein X is bromine for 4a and X is iodine for 4b) with a cyclic amide of Formula 5 in the presence of copper (I) iodide with a diamine ligand, e.g. trans-N,N′-Dimethylcyclohexane-1,2-diamine or tetramethylethylenediamine (TMEDA), and potassium phosphate (K₃PO₄) in an appropriate solvent. The solvent can be, for example, N,N-dimethylformamide, acetonitrile, tetrahydrofuran or dioxane, optionally with water as a cosolvent. A similar copper-mediated coupling can also be carried out under Chan-Lam conditions where a boronic acid of Formula 4c (i.e. a compound of Formula 4 wherein X is B(OH)₂) is coupled with a compound of Formula 5 in the presence of copper II acetate (Cu(II)AC₂) and pyridine in dichloromethane. Alternatively, this cross-coupling can also be carried out with a compound of Formula 4c and a compound for Formula 5 under the well-documented Buchwald-Hartwig amination protocol involving palladium-mediation with a suitable phosphine ligand, either as part of the pre-catalyst or as an additive in an appropriate solvent such as tetrahydrofuran, toluene or dichloromethane. In some cases, an auxiliary base, i.e. sodium tert-butoxide or cesium carbonate, is used in the reaction. Examples of palladium catalysts suitable for this transformation include but are not limited to tetrakis(triphenylphosphine) palladium(0) [Pd(PPh₃)₄], bistriphenylphosphine palladium chloride [PdCl₂(PPh₃)₂], palladium(II) chloride-tris(2-methylphenyl)phosphine [PdCl₂[P(o-Tol)₃]₂] or [1,1′bis(diphenylphosphino) ferrocene] dichloropalladium(II) [Pd(dppf)Cl₂]. Finally, this cross-coupling can also be accomplished with palladium acetate [Pd(OAc)₂] or tris(dibenzylideneacetone) dipalladium(0) [Pd₂(dba)] optionally used in combination with a suitable phosphine ligand with a base such as sodium tert-butoxide in toluene or cesium carbonate in N,N-dimethylformamide.

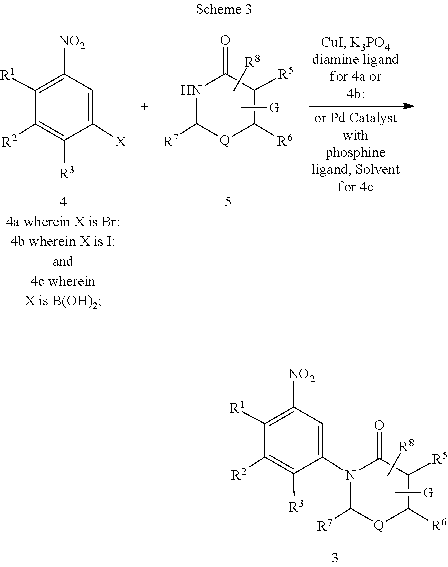

As illustrated in Scheme 4, nitrobenzenes of Formula 4 can be prepared by nitration of a substituted benzene of Formula 6 in a mixture of nitric acid and sulfuric acid at temperatures ranging from 0° C. to ambient temperature to afford nitrobenzenes of Formula 4. Other sources of nitronium ion for this nitration include nitronium tetrafluoroborate, acetyl nitrate, guanidinium nitrate, used in an appropriate solvent such as tetramethylene sulfone. Substituted benzenes of Formula 6 are, in some cases, commercially available and in other cases readily prepared by established methods from the literature. It is recognized that nitration of some substituted benzenes of Formula 6 can give rise to regioisomeric mixture of nitrobenzenes that require separation by chromatography or fractional crystallization techniques.

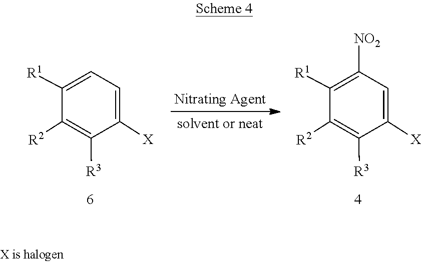

Alternatively, a nitrobenzene of Formula 4a (i.e. a compound of Formula 4 wherein X is bromine) or a nitrobenzene of Formula 4b (i.e. a compound of Formula 4 wherein X is idodine) can be prepared by halogenation of a substituted nitrobenzene of Formula 7 with an appropriate halogenating reagent, such as bromine, iodine, N-bromosuccinimide or N-iodosuccinimide, in an appropriate solvent, such as acetic acid, dichloromethane, carbon tetrachloride, chloroform, acetonitrile or N,N-dimethylformamide by established methods as shown in Scheme 5. Iodobenzenes of Formula 4b can also be made from benzenes of Formula 7 by treating with 2,2,6,6-tetramethylpiperidylzincchloride-LiCl (TMPZnCl·LiCl) in tetrahydrofuran or dioxane, followed by the addition of iodine and a mixture of nitric acid and sulfuric acid at temperatures ranging from 0° C. to ambient temperature. Bromo and iodo benzenes of Formulae 4a and 4b can be lithiated with an alkyl lithium reagent, preferably n-butyl lithium, in tetrahydrofuran or dioxane typically at temperatures generally ranging from −78° C. to 0° C., followed by addition of trimethyl boroxine and subsequent acidic hydrolysis to afford the corresponding aryl boronic acids of Formula 4c (i.e. a compound of Formula 4 wherein X is B(OH)₂). Conversion of aryl halides to aryl boronic acids is a well-established synthetic transformation in the organic chemistry literature.

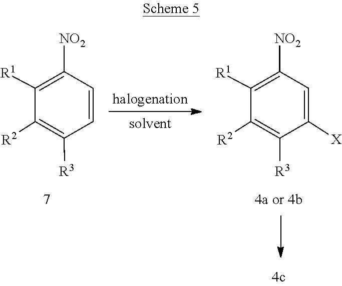

As shown in Scheme 6, a cyclic amide of Formula 5a can be made from hydroxy-substituted N-protected cyclic amides of Formula 8, where PG represents a protecting group such as a Cbz (benzyloxycarbonyl) or BOC (tert-butyloxycarbonyl) group. Alkylating the compound of Formula 8 with an appropriate alkylating agent, in the presence of a base, such as sodium hydride, potassium tert-butoxide or sodium methoxide, in a solvent like tetrahydrofuran or dioxane at temperatures generally ranging from 0° C. to reflux temperature of the solvent affords a compound of Formula 9. The N-protecting group CBZ can then be removed by catalytic hydrogenation (generally under hydrogen in the presence of palladium-on-carbon in methanol or ethanol) to give a compound of Formula 5a. The N-protecting group BOC can be removed by trifluoroacetic acid to provide a compound of Formula 5a. Intermediate cyclic amides of Formula 9 can also be made from cyclic amides of Formula 10 where LG represents an appropriate leaving group such as a halogen (i.e. chlorine, bromine or iodine) or mesylate. Reacting a compound of Formula 10 with a nucleophile of Formula R¹⁰OH, in the presence of a base such as sodium hydride, potassium tert-butoxide or sodium methoxide, in a solvent such as tetrahydrofuran or dioxane at temperatures generally ranging from 0° C. to reflux temperature of the solvent afford a compound of Formula 9.

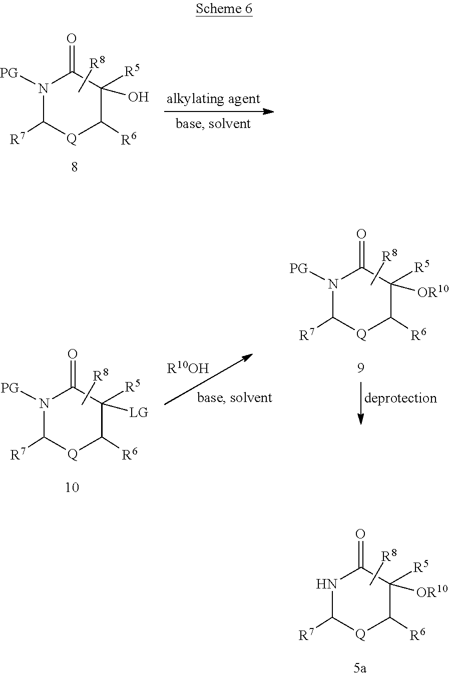

A compound of Formula 3a (i.e. a compound of Formula 3, wherein G is OR¹⁰) can also be accessed by the synthetic route outlined in Scheme 7. Cross-coupling of a meta-bromo or meta-iodo substituted nitrobenzene of Formula 4a or 4b (i.e. a compound of Formula 4, wherein X is bromine or iodine) with a hydroxy-substituted cyclic amide of Formula 11 by the same methods described for the cross-coupling in Scheme 3, affords a compound of Formula 12 with a free hydroxy group. Alkylation of 12 with an appropriate alkylating agent in the presence of a base such as sodium hydride, potassium tert-butoxide or sodium methoxide in a solvent such as tetrahydrofuran or dioxane at temperatures generally ranging from 0° C. to reflux temperature of the solvent, gives a compound of Formula 3a. Alternatively, a compound of Formula 3a can be made in some cases by the method outlined in Scheme 8. Cross-coupling of an unprotected cyclic amide of Formula 13 with a substituted nitrobenzene of Formula 4 under the same cross-coupling conditions as described in Scheme 3, can give a compound of Formula 14. The unprotected cyclic amide of Formula 13 contains both a suitable leaving group LG, wherein LG is bromine, chlorine or iodine, and a free amide NH group. Displacement of the leaving group LG on 14 with a sodium or potassium alkoxide (NaOR¹⁰ or KOR¹⁰) in a suitable solvent such as tetrahydrofuran, dioxane, methanol, ethanol, dimethylsulfoxide or N,N-dimethylforamide provides a compound of Formula 3a.

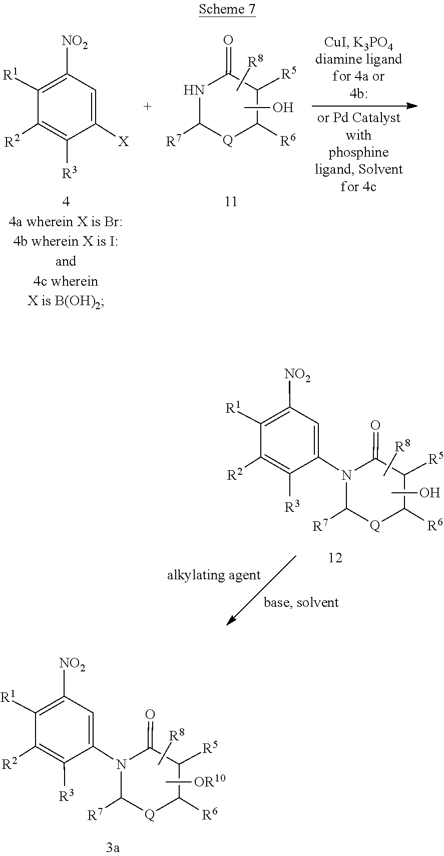

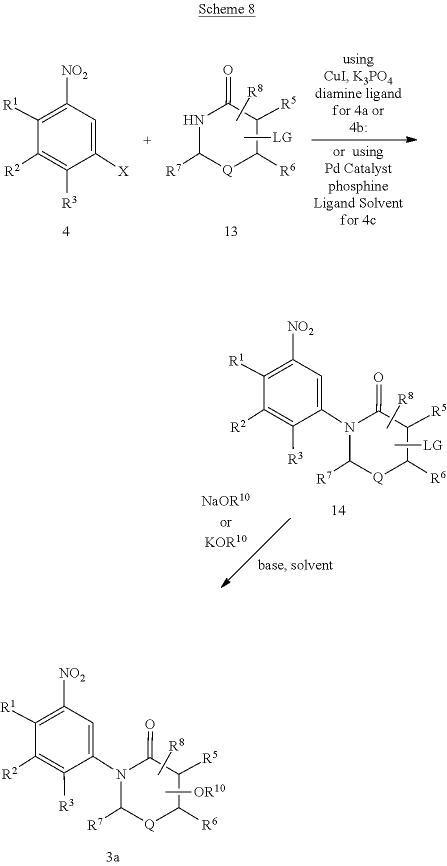

Alternatively, a compound of Formula 3b (i.e. a compound of Formula 3, wherein G is SR¹⁰) can be made as outlined in Scheme 9. Displacement of the leaving group LG on a compound of Formula 14 with a sodium or potassium thiol reagent (NaSR¹⁰ or KSR¹⁰) in a suitable solvent such as tetrahydrofuran, dioxane, acetonitrile or N,N-dimethylformamide at temperatures ranging 0° C. to the reflux temperature of the solvent can afford a compound of Formula 3b. Oxidation of the sulfur with an appropriate oxidizing agent such as meta-chloroperoxybenzoic (MCPBA), sodium periodate or Oxone can provide the corresponding sulfoxide (SOR¹⁰) and sulfone (SO₂R¹⁰).

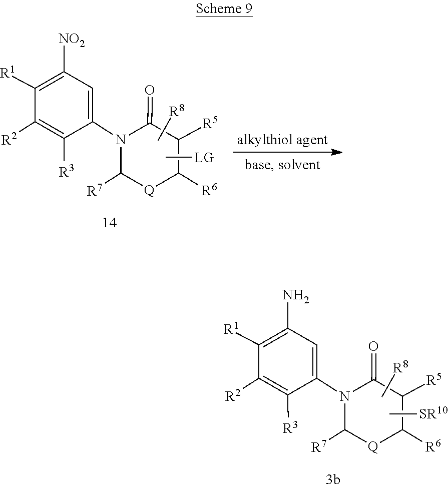

A method for making a compound of Formula 5b (i.e. a compound of Formula 5 wherein X is O) or a compound of Formula 5c (i.e. a compound of Formula 5 wherein X is S) is outlined in Scheme 10. Based on a known method (see Eur. J. Org. Chem. 2020, 3013-3018), heating a BOC (tert-butyloxycarbonyl)-protected cyclic amide of Formula 15 with t-butoxy bis-(dimethylamino)methane in toluene or xylene at the reflux temperature gives the corresponding enamine adduct 16. A compound of 16 can be reacted with sodium azide in the presence of chlorosulfonyl benzoic acid and potassium carbonate, in aqueous acetonitrile, to generate the diazo compound 17. A compound of Formula 17 can undergo a rhodium-catalyzed carbenoid insertion into an alcohol (R¹⁰OH) O—H bond or thiol (R¹⁰SH)S—H bond to generate an OR¹⁰ or SR¹⁰ substituted BOC-protected cyclic amide of Formula 18b wherein X is O or Formula 18c wherein X is S. Removal of the BOC-protecting group under acidic conditions, generally in trifluoroacetic acid, gives the free cyclic amide of Formula 5b wherein X is O or Formula 5c wherein X is S. This is a particularly useful method for introducing OR¹⁰ and SR¹⁰ groups where the R¹⁰ moiety may be a branched-chain, cyclic or bulky substituent.

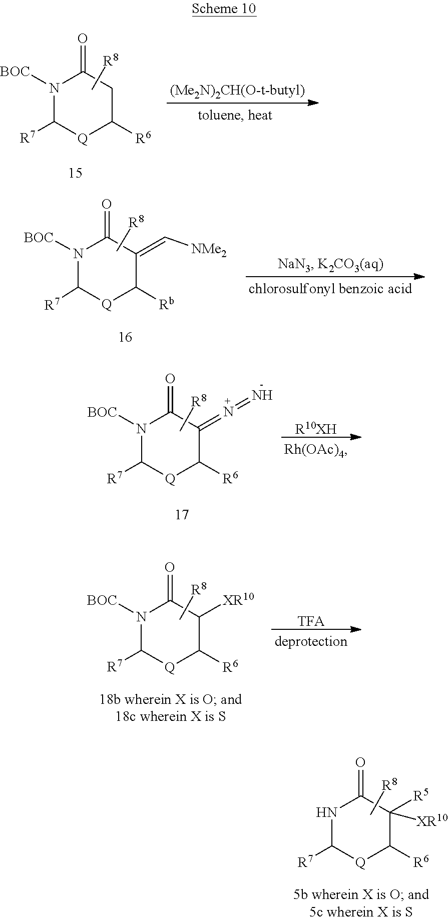

Compounds of Formula 1 where R⁴ is C(═O)R¹⁴, C(═S)R¹⁴, CO₂R¹⁴, C(═O)SR¹⁴, S(O)₂R¹⁴, CONR¹³R¹⁴, S(O)₂NR¹³R¹⁴, CH₂OC(═O)NR¹³R¹⁴, CH₂OC(═O)OR¹⁴ or CH₂O(C═O)R¹⁴ can be made by reaction of a sulfonanilide of Formula 1 where R⁴ is hydrogen with an appropriately substituted acyl halide, thioacyl halide, carbamoyl halide, sulfonyl halide, sulfamoyl halide, acyloxymethyl halide (i.e. ClCH₂O(C═O)R¹⁴) or a similar halide, or other capping agents in the presence of a base such as triethylamine, pyridine, diisopropylethyl amine (Hunig's Base) or potassium carbonate in a solvent including but not limited to tetrahydrofuran, dioxane, dichloromethane, acetonitrile or N,N-dimethylformamide (Scheme 11).

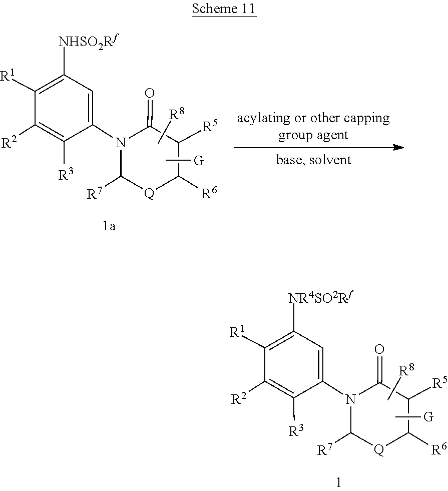

Compounds of Formula 1c (i.e. a compound of Formula 1 where R⁴ is H, and G and R⁵ are taken together to form N—OR¹⁵ where R¹⁵ is not H) can be prepared by treatment of a compound of Formula 1d (i.e. a compound of Formula 1 where R⁴ is H, and G and R⁵ are taken together to form N—OH) with an appropriate alkylating agent, in the presence of a base such as potassium tert-butoxide or sodium hydride, in a solvent like tetrahydrofuran at temperatures generally ranging from 0° C. to the reflux temperature of the solvent.

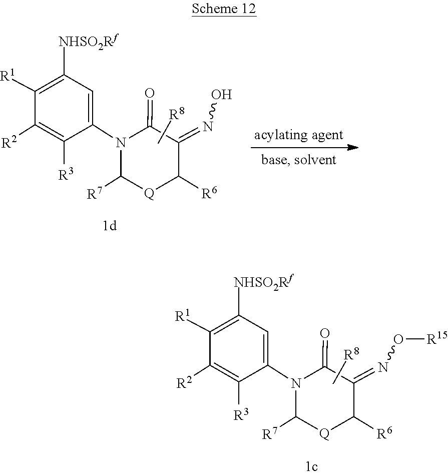

Compounds of Formula 1d (i.e. a compound of Formula 1 where R⁴ is H, and G and R⁵ are taken together to form N—OH) can be prepared by treatment of a compound of Formula 19, with a strong base such as, but not limited to sodium bis(trimethylsilyl)amide, lithium bis(trimethylsilyl)amide, potassium bis(trimethylsilyl)amide or lithium diisopropylamide and a nitrosylating agent, for example an alkyl nitrite such as, but not limited to isopentyl nitrite or tert-butyl nitrite. The reactions are typically performed in a solvent such as tetrahydrofuran at temperatures ranging from approximately −78° C. to 50° C. Representative examples may be found in Chem. Pharm. Bull. 1986, vol. 34, pp. 2732-2742 and Org. Lett. 2021, vol. 23, pp. 5394-5399. Compounds of Formula 19 can be prepared using the preceding description.

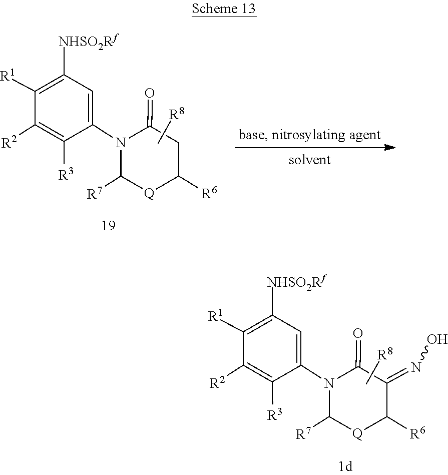

It is recognized by one skilled in the art that various functional groups can be converted into others to provide different compounds of Formula 1. For a valuable resource that illustrates the interconversion of functional groups in a simple and straightforward fashion, see Larock, R. C., Comprehensive Organic Transformations: A Guide to Functional Group Preparations, 2nd Ed., Wiley-VCH, New York, 1999. For example, intermediates for the preparation of compounds of Formula 1 may contain aromatic nitro groups, which can be reduced to amino groups, and then be converted via reactions well known in the art such as the Sandmeyer reaction, to various halides, providing compounds of Formula 1. The above reactions can also in many cases be performed in alternate order.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd Ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical organometallic, oxidation, and reduction reactions to add substituents or modify existing substituents.

Without further elaboration, it is believed that one skilled in the art using the preceding description can utilize the present invention to its fullest extent. The following non-limiting Examples are illustrative of the invention. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “q” means quartet, “m” means multiplet, “dd” means doublet of doublets, “ddd” means doublet of doublets of doublets, “dt” means doublet of triplets, and “br s” means broad singlet. Mass spectra (MS) are reported as the molecular weight of the highest isotopic abundance parent ion (M+1) formed by addition of H+(molecular weight of 1) to the molecule or (M−1) formed by the loss of H+(molecular weight of 1) from the molecule, observed by using liquid chromatography coupled to a mass spectrometer (LCMS) using either atmospheric pressure chemical ionization (AP+) where “amu” stands for unified atomic mass units.

The following non-limiting Examples are meant to be illustrative of the present processes for preparing compounds of Formula 1 and corresponding intermediates. All NMR spectra are reported in CDCl₃ at 500 MHz downfield from tetramethyl silane unless otherwise indicated.

### Synthesis Example 1

### Preparation of [[5-[3-(Cyclopentyloxy)-2-oxo-1-pyrrolidinyl]-2,4-dimethylphenyl][(trifluoromethyl)sulfonyl]amino]methyl 2,2-dimethylpropanoate (Compound 5)

### Step A: Preparation of tert-butyl 3-(cyclopentoxy)-2-oxo-pyrrolidine-1-carboxylate

To a solution of tert-butyl 3-diazo-2-oxopyrolidine-1-carboxylate (300 mg, 1.42 mmol) and cyclopentanol (0.26 mL, 2.84 mmol) in dichloromethane (5 mL) was added dirhodium tetraacetate (19 mg, 3 mol %). The mixture was stirred at room temperature for 2 h and then concentrated under reduced pressure. The residue was purified by column chromatography (0-60% ethyl acetate in hexanes gradient on silica) to afford the desired product (342 mg) as a clear oil.

¹H NMR (CDCl₃) δ 1.53 (s, 9H), 1.55-1.62 (m, 4H), 1.71-1.82 (m, 4H), 1.86-1.98 (m, 1H) 2.23-2.29 (m, 1H), 3.52 (ddd, J=10.92, 8.08, 7.17 Hz, 1H), 3.79 (ddd, J=10.88, 8.51, 3.78 Hz, 1H), 4.05 (t, J=7.88 Hz, 1H), 4.36-4.41 (m, 1H).

### Step B: Preparation of 3-(cyclopentoxy)pyrrolidin-2-one

To a solution of tert-butyl 3-(cyclopentoxy)-2-oxo-pyrrolidine-1-carboxylate (i.e. the product of Step A) (342 mg, 1.27 mmol) in dichloromethane (5 mL) was added trifluoroacetic acid (0.29 mL, 3.81 mmol). The reaction mixture was stirred at room temperature for 2 h before quenched with NaHCO₃ (aq.) and extracted with dichloromethane. Combined organic layers were dried with magnesium sulfate and concentrated under reduced pressure to afford 3-(cyclopentoxy)pyrrolidin-2-one (191 mg) as a clear oil and used without further purification.

¹H NMR (CDCl₃) δ 1.48-1.62 (m, 4H), 1.64-1.86 (m, 4H), 2.01-2.10 (m, 1H), 2.37-2.46 (m, 1H), 3.27 (dt, J=9.50, 7.23 Hz, 1H), 3.41 (td, J=8.99, 3.63 Hz, 1H), 4.02 (t, J=7.49 Hz, 1H), 4.30-4.38 (m, 1H), 6.03 (br s, 1H).

### Step C: Preparation of 3-(cyclopentoxy)-1-(2,4-dimethyl-5-nitro-phenyl)pyrrolidin-2-one

To a 25 mL scintillation vial with septum, copper(I) iodide (45 mg, 25 mol %), potassium carbonate (390 mg, 2.82 mmol), 3-(cyclopentoxy)pyrrolidin-2-one (i.e. the product of Step B) (191 mg, 1.13 mmol) and 1-bromo-2,4-dimethyl-5-nitrobezene (216 mg, 0.94 mmol) were added. The reaction vial was purged with nitrogen gas before dioxane (5 mL) and trans-N,N′-dimethyl-cyclohexane-1,2-diamine (0.074 mL, 50 mol %) were added to the reaction vial via syringe. The reaction mixture was stirred under nitrogen at 100° C. overnight, then diluted with ethyl acetate and filtered through a pad of Celite® diatomaceous earth filter aid. The resulting filtrate was dried over magnesium sulfate and concentrated under reduced pressure to a residue. The residue was purified by column chromatography (0-60% ethyl acetate in hexanes gradient on silica) to afford the desired product (279 mg) as a clear oil.

¹H NMR (CDCl₃) δ:1.49-1.61 (m, 3H), 1.67-1.86 (m, 5H), 2.17 (ddt, J=13.00, 8.04, 6.42, 6.42 Hz, 1H), 2.27 (s, 3H), 2.46-2.54 (m, 1H), 2.60 (s, 3H), 3.64 (ddd, J=9.65, 7.29, 6.38 Hz, 1H), 3.73 (ddd, J=9.62, 8.04, 4.57 Hz, 1H), 4.18-4.21 (m, 1H), 4.38-4.49 (m, 1H), 7.24 (s, 1H), 7.86 (s, 1H)

### Step D: Preparation of 1-(5-amino-2,4-dimethyl-phenyl)-3-(cyclopentoxy)pyrrolidin-2-one

To a stirred solution of 3-(cyclopentoxy)-1-(2,4-dimethyl-5-nitro-phenyl)pyrrolidin-2-one (i.e. the product of Step C) (278 mg, 0.87 mmol) in ethyl acetate (4 mL) was added a solution of ammonium chloride (93 mg, 1.75 mmol) in water (1 mL). Iron powder (146 mg, 2.62 mmol) was then added and stirred at 80° C. under nitrogen overnight. The mixture was cooled to room temperature, diluted with ethyl acetate and filtered through a pad of Celite® diatomaceous earth filter aid. The filtrate was concentrated under reduced pressure to afford the title compound (275 mg) and used without further purification.

¹H NMR (CDCl₃) δ 1.42-1.62 (m, 3H), 1.66-1.86 (m, 5H), 2.04-2.25 (m, 7H), 2.38-2.51 (m, 1H), 3.53 (ddd, J=9.77, 7.41, 6.46 Hz, 1H), 3.65 (ddd, J=9.81, 8.16, 4.41 Hz, 1H), 4.16-4.18 (m, 1H), 4.37-4.53 (m, 1H), 6.48 (s, 1H) 6.92 (s, 1H).

### Step E: Preparation of N-[5-[3-(cyclopentyloxy)-2-oxo-1-pyrrolidinyl]-2,4-dimethylphenyl]-1,1,1-trifluoro-N-[(trifluoromethyl)sulfonyl]methanesulfonamide

To a stirred solution of 1-(5-amino-2,4-dimethyl-phenyl)-3-(cyclopentoxy)pyrrolidin-2-one (i.e. the product of Step D) (275 mg, 0.95 mmol) in dichloromethane (4.8 mL) was added triethylamine (0.279 mL, 2.00 mmol). The mixture was cooled to −78° C., then trifluoromethanesulfonic anhydride (0.34 mL, 2.00 mmol) was added dropwise. The reaction mixture was then stirred at room temperature for 1 h before quenched with aqueous NaHCO₃ solution and extracted with dichloromethane. The combined organic layers were dried with magnesium sulfate, concentrated under reduced pressure and purified by column chromatography (0-60% ethyl acetate in hexanes gradient on silica) to afford the title compound (380 mg).

¹H NMR (CDCl₃) δ 1.50-1.61 (m, 3H), 1.68-1.89 (m, 5H), 2.16 (ddt, J=13.10, 8.18, 6.54, 6.54 Hz, 1H), 2.25 (s, 3H), 2.39 (s, 3H), 2.45-2.55 (m, 1H), 3.56-3.63 (m, 1H), 3.66-3.73 (m, 1H), 4.20 (dd, J=7.41, 6.62 Hz, 1H), 4.43 (tt, J=5.87, 3.59 Hz, 1H), 7.08 (s, 1H), 7.26 (s, 1H).

### Step F: Preparation of N-[5-[3-(cyclopentyloxy)-2-oxo-1-pyrrolidinyl]-2,4-dimethylphenyl]-1,1,1-trifluoromethanesulfonamide

To a stirred solution of N-[5-[3-(cyclopentyloxy)-2-oxo-1-pyrrolidinyl]-2,4-dimethylphenyl]-1,1,1-trifluoro-N-[(trifluoromethyl)sulfonyl]methanesulfonamide (i.e. the product of Step E) (380 mg, 0.69 mmol) in dioxane (6.8 mL) was added 1 N aqueous sodium hydroxide solution (0.72 mL, 0.72 mmol) dropwise. The reaction mixture was stirred at room temperature for 3 h, then neutralized with 1 N aqueous hydrogen chloride solution and extracted with dichloromethane. The combined organic layers were dried with magnesium sulfate, concentrated under reduced pressure and purified by column chromatography (0-50% ethyl acetate in hexanes gradient, on silica) to afford the title compound (160 mg) as a white solid.

¹H NMR (CDCl₃) δ 1.50-1.60 (m, 2H), 1.65-1.86 (m, 6H), 2.12-2.19 (m, 7H), 2.43-2.52 (m, 1H), 3.54 (ddd, J=10.01, 7.49, 6.46 Hz, 1H), 3.66 (ddd, J=10.01, 8.28, 4.41 Hz, 1H), 4.24 (dd, J=7.72, 6.31 Hz, 1H), 4.46-4.53 (m, 1H), 6.87 (s, 1H), 7.03 (s, 1H), 8.65 (br s, 1H).

### Step G: Preparation of [[5-[3-(cyclopentyloxy)-2-oxo-1-pyrrolidinyl]-2,4-dimethylphenyl][(trifluoromethyl)sulfonyl]amino]methyl 2,2-dimethylpropanoate

To a stirred solution of N-[5-[3-(cyclopentyloxy)-2-oxo-1-pyrrolidinyl]-2,4-dimethylphenyl]-1,1,1-trifluoromethanesulfonamide (i.e. the product of Step F) (70 mg, 0.17 mmol) in dichloromethane (5 mL) was added triethylamine (0.058 mL, 0.42 mmol) and chloromethyl 2,2-dimethylpropanoate (0.048 mL, 0.33 mmol). The reaction mixture was stirred overnight at 45-50° C. before concentrated under reduced pressure. The residue was purified by column chromatography (0-100% ethyl acetate in hexane gradient, on silica) to afford the title compound (75 mg) as a clear oil.

¹H NMR (CDCl₃) δ 1.20 (d, J=3.63 Hz, 9H), 1.50-1.60 (m, 2H), 1.66-1.87 (m, 6H), 2.10-2.18 (m, 1H), 2.21 (d, J=9.62 Hz, 3H), 2.38 (s, 3H), 2.41-2.52 (m, 1H), 3.52-3.57 (m, 1H), 3.64-3.75 (m, 1H), 4.13-4.18 (m, 1H), 4.41-4.45 (m, 1H), 5.42 (t, J=10.64 Hz, 1H), 5.70 (t, J=11.59 Hz, 1H), 7.05 (d, J=17.50 Hz, 1H), 7.22 (s, 1H).

### Synthesis Example 2

### Preparation of N-[2,4-dimethyl-5-[2-oxo-3-(2-propyn-1-yloxy)-1-pyrrolidinyl]phenyl]-1,1,1-trifluoromethanesulfonamide (Compound 1)

### Step A: Preparation of 1-(2,4-dimethyl-5-nitro-phenyl)-3-hydroxy-pyrrolidin-2-one

To a solution of 1-bromo-2,4-dimethyl-5-nitrobezene (2.50 g, 10.86 mmol) in 1, 4-dioxane (20 mL) was added 3-hydroxypyrrolidin-2-one (2.74 g, 27.17 mmol), K₂CO₃ (4.50 g, 32.60 mmol), copper(I) iodide (2.06 g, 10.86 mmol) and N,N-Dimethylethylenediamine (DMEDA) (2.3 mL, 21.73 mmol) at room temperature. The reaction mixture was degassed under N₂ for 10 min and then stirred at 110° C. for 16 h. The reaction mixture was filtered through Celite® diatomaceous earth filter aid and washed with ethyl acetate (50 mL). The filtrate was evaporated under reduced pressure and triturated with n-pentane (25 mL), and diethyl ether (5 mL) to give the desired product (2.2 g) as off-white solid.

¹H NMR (CDCl₃) δ 7.87 (s, 1H), 7.26 (s, 1H), 5.54-4.99 (t, 1H), 3.76-3.65 (m, 2H), 2.94 (br, 1H), 2.66-2.63 (m, 1H), 2.60 (s, 3H), 2.27 (s, 3H), 2.26-2.20 (m, 1H).

### Step B: Preparation of 1-(2,4-dimethyl-5-nitro-phenyl)-3-prop-2-ynoxy-pyrrolidin-2-one

To a solution of 1-(2,4-dimethyl-5-nitro-phenyl)-3-hydroxy-pyrrolidin-2-one (i.e. the product of Step A) (1.5 g, 6 mmol) in THF (30 mL) was added NaH (0.432 g, 18 mmol, 60%) and propargyl bromide (1.36 mL, 18 mmol) at 0° C. The reaction mixture was stirred at room temperature for 16 h. The reaction mixture was quenched with saturated aqueous NH₄Cl solution (10 mL) and extracted with ethyl acetate (25 mL×2). Combined organic layers were dried over anhydrous Na₂SO₄. The solvent was concentrated under reduced pressure to give the crude product. The cruder product was charged on silica gel column. Elution of the column with 30% ethyl acetate/petroleum ether gave the desired product (500 mg) as a light yellow solid.

LCMS (M+1)=289.

### Step C: Preparation of 1-(5-amino-2,4-dimethylphenyl)-3-(2-propyn-1-yloxy)-2-pyrrolidinone

To a solution of 1-(2,4-dimethyl-5-nitro-phenyl)-3-prop-2-ynoxy-pyrrolidin-2-one (i.e. the product of Step B) (0.400 g, 1.38 mmol) in ethanol (16 mL) and water (4 mL) was added iron (power, 0.387 g, 6.94 mmol) and NH₄Cl (0.074 g, 1.38 mmol). The reaction mixture was heated to the reflux temperature at 80° C. for 3 h. After completion of the reaction, the reaction mixture was filtered through Celite® diatomaceous earth filter aid and washed with ethyl acetate (25 mL). The filtrate was evaporated under reduced pressure to give the crude product (0.240 g) as an off-white solid which was used in the next step.

LCMS (M+1)=259.

### Step D: Preparation of N-[2,4-dimethyl-5-[2-oxo-3-(2-propyn-1-yloxy)-1-pyrrolidinyl]phenyl]-1,1,1-trifluoromethanesulfonamide

To a solution of 1-(5-amino-2,4-dimethylphenyl)-3-(2-propyn-1-yloxy)-2-pyrrolidinone (i.e. the product of Step C) (0.210 g, 0.81 mmol) in dichloromethane (10 mL) was added triethylamine (0.2 mL, 1.62 mmol) and Trifluoromethanesulfonic anhydride (Tf₂O) (0.08 mL, 0.48 mmol) at −78° C. The reaction mixture was stirred at room temperature for 1 h. After completion of the reaction, the reaction mixture was quenched with water (20 mL) and extracted with dichloromethane (20 mL×2). The organic layer was separated and washed with brine (10 mL) and concentrated under reduced pressure to give the crude compound which was loaded on silica gel column. Elution of the column with 30% ethyl acetate/petroleum ether gave the desired product (80 mg) as an off-white solid.

¹H NMR (CDCl₃) δ 7.99 (br, 1H), 7.06 (s, 1H), 6.97 (s, 1H), 4.65-4.53 (m, 2H), 4.46-4.42 (t, 1H), 3.70-3.57 (m, 2H), 2.59-2.56 (m, 1H), 2.50-2.49 (t, 1H), 2.26-2.24 (m, 1H), 2.21 (s, 3H), 2.16 (s, 3H).

### Synthesis Example 3

### Preparation of N-[5-[3-(cyclopropoxy)-2-oxo-1-pyrrolidinyl]-2,4-dimethylphenyl]-1,1,1-trifluoromethanesulfonamide (also known as N-[5-[3-(cyclopropoxy)-2-oxo-pyrrolidin-1-yl]-2,4-dimethylphenyl]-1,1,1-trifluoromethanesulfonamide (Compound 3)

### Step A: Preparation of tert-butyl 3-(cyclopropoxy)-2-oxo-pyrrolidine-1-carboxylate

To a solution of tert-butyl 3-diazo-2-oxopyrolidine-1-carboxylate (2 g, 9.47 mmol) and cyclopropanol (0.82 g, 14.21 mmol) in dichloromethane (20 mL) was added dirhodium tetraacetate (41 mg, 0.01 mmol). The mixture was stirred at room temperature for 1 h. Analysis by thin layer chromatography (50% ethyl acetate/petroleum ether) showed completion of the reaction. The reaction mixture was filtered through Celite® diatomaceous earth filter aid; and the filtrate was evaporated under reduced pressure to obtain the crude product. The crude product was loaded on a silica gel column. Elution of the column with 30% ethyl acetate/petroleum ether gave the pure desired product (0.680 g) as off-white solid.

¹H NMR (CDCl₃) δ 4.17-4.13 (t, 1H), 3.82-3.77 (m, 2H), 3.57-3.52 (m, 1H), 2.28-2.27 (m, 1H), 1.96-1.91 (m, 1H), 1.53 (s, 9H), 0.72-0.49 (m, 4H).

### Step B: Preparation of 3-(cyclopropoxy)pyrrolidin-2-one

To a solution of tert-butyl 3-(cyclopropoxy)-2-oxo-pyrrolidine-1-carboxylate (i.e. the product of Step A) (0.680 g, 2.61 mmol) in dichloromethane (10 mL) was added trifluoroacetic acid (0.89 g, 7.84 mmol) dropwise. The reaction mixture was stirred at room temperature for 4 h. Analysis by thin layer chromatography (45% ethyl acetate/petrolium ether ether showed completion of the reaction. The reaction mixture was evaporated under reduced pressure to obtain the crude product. The crude product was co-distilled with CHCl₃ (10 mL×2) to get 3-(cyclopropoxy)pyrrolidin-2-one (0.6 g) as a clear oil liquid.

¹H NMR (CDCl₃) δ 7.69 (br, 1H), 4.3-4.26 (m, 1H), 3.71-3.68 (m, 1H), 3.56-3.50 (m, 1H), 3.43-3.37 (m, 1H), 2.52-2.44 (m, 1H), 2.16-2.07 (m, 1H), 0.74-0.54 (m, 4H).

### Step C: Preparation of 3-(cyclopropoxy)-1-(2,4-dimethyl-5-nitro-phenyl)pyrrolidin-2-one

To a solution of 3-(cyclopropoxy)pyrrolidin-2-one (i.e. the product of Step B) (0.6 g, 4.25 mmol) in dioxane in a sealed vessel was added 1-bromo-2,4-dimethyl-5-nitrobezene (2.12 g, 8.5 mmol), K₂CO₃ (2.5 g, 17.02 mmol) and N,N-Dimethylethylenediamine (DMEDA) (0.81 g, 8.5 mmol). The reaction was degassed with N₂ gas for 5 min. Copper(I) iodide (0.875 g, 4.2 mmol) was added to the reaction mixture and the reaction mixture was heated to the reflux temperature at 110° C. for 12 h. The reaction mixture was diluted with ethyl acetate and filtered through a pad of Celite® diatomaceous earth filter aid. The resulting filtrate was concentrated under reduced pressure to afford a residue. The residue was purified by column chromatography (30% ethyl acetate in petroleum ether on silica) to afford the desired product (0.650 g) as a white solid.

¹H NMR (CDCl₃) δ 7.86 (s, 1H), 7.26 (s, 1H), 4.32-4.28 (t, 1H), 3.82-3.79 (m, 1H), 3.75-3.70 (m, 2H), 2.60 (s, 3H), 2.28 (s, 3H), 2.58-2.53 (m, 1H), 2.23-2.18 (m, 1H), 0.79-0.54 (m, 4H).

### Step D: Preparation of 1-(5-amino-2,4-dimethylphenyl)-3-(cyclopropyloxy)-2-pyrrolidinone

To a solution of 3-(cyclopropoxy)-1-(2,4-dimethyl-5-nitro-phenyl)pyrrolidin-2-one (i.e. the product of Step C) (0.610 g, 2.10 mmol) in ethanol (5 mL) and water (5 mL) was added iron (powder, 0.587 g, 10.55 mmol) and NH₄Cl (0.336 g, 6.310 mmol). The reaction mixture was heated at 80° C. for 2 h. After completion of the reaction, the reaction mixture was filtered through Celite® diatomaceous earth filter aid and washed with ethyl acetate (25 mL). The filtrate was evaporated under reduced pressure to give the crude product which was loaded on silica gel column. Elution of the column with 40% ethyl acetate/petroleum ether gave the desired product (0.49 g) as an off-white solid.

¹H NMR (CDCl₃) δ 6.93 (s, 1H), 6.46 (s, 1H), 4.29-4.26 (t, 1H), 3.83-3.80 (m, 1H), 3.66-3.55 (m, 2H), 2.49-2.44 (m, 1H), 2.18-2.12 (m, 1H), 2.11 (s, 3H), 2.08 (s, 3H), 0.76-0.52 (m, 4H).

### Step E: Preparation of N-[5-[3-(cyclopropoxy)-2-oxo-1-pyrrolidinyl]-2,4-dimethylphenyl]-1,1,1-trifluoromethanesulfonamide (also known as N-[5-[3-(cyclopropoxy)-2-oxo-pyrrolidin-1-yl]-2,4-dimethyl-phenyl]-1,1,1-trifluoro-methanesulfonamide)

To a solution of 1-(5-amino-2,4-dimethylphenyl)-3-(cyclopropyloxy)-2-pyrrolidinone (i.e. the product of Step D) (350 mg, 1.34 mmol) in dichloromethane (10 mL) was added triethylamine (0.37 mL, 2.26 mmol) and Tf₂O (0.34 mL, 2.01 mmol) at −20° C. The reaction mixture was stirred at room temperature for 3 h. Analysis by thin layer chromatography (50% ethyl acetate/petroleum ether) showed completion of the reaction. The reaction mixture was quenched with water (50 mL) and extracted with diclhloromethane (50 mL×2). The organic layer was separated, washed with brine (25 mL) and dried over Na₂SO₄. The solvent was evaporated and loaded on silica gel column. Elution of the column with 20% ethyl acetate/petroleum ether gave the desired product (140 mg) as an off-white solid.

¹H NMR (CDCl₃) δ 8.12 (s, 1H), 7.06 (s, 1H), 6.95 (s, 1H), 4.35-4.31 (t, 1H), 3.89-3.84 (m, 1H), 3.69-3.55 (m, 2H), 2.55-2.48 (m, 1H), 2.22 (s, 3H), 2.17 (s, 3H), 2.17 (m, 1H), 0.81-0.76 (m, 1H), 0.68-0.62 (m, 3H).

### Synthesis Example 4

### Preparation of 1,1,1-trifluoro-N-[5-[3-(hydroxyimino)-2-oxo-1-pyrrolidinyl]-2,4-dimethylphenyl]methanesulfonamide (Compound 10)

### Step A: Preparation of 1-(2,4-dimethyl-5-nitro-phenyl)pyrrolidin-2-one

To a stirred solution of 1-bromo-2,4-dimethyl-5-nitro-benzene (5 g, 21.7 mmol) in 1,4-dioxane (50 mL) was added pyrrolidin-2-one (4.6 g, 54.1 mmol), potassium carbonate (8.9 g, 64.4 mmol), copper(I) iodide (3.9 g, 20.5 mmol) and N,N′-dimethylethylenediamine (3.82 g, 43.3 mmol). The mixture was sparged with nitrogen gas for 10 min then stirred at 130° C. for 16 h. The mixture was filtered through a pad of Celite, rinsing with ethyl acetate (50 mL). The filtrate was concentrated under reduced pressure and triturated with n-pentane (25 mL) and diethyl ether (5 mL) to give the title compound as an off white solid (5 g).

¹H NMR (CDCl₃) δ 7.87 (s, 1H), 7.24 (s, 1H), 3.78-3.75 (m, 2H), 2.61-2.57 (m, 5H), 2.30-2.24 (m, 5H).

### Step B: Preparation of 1-(5-amino-2,4-dimethyl-phenyl)pyrrolidin-2-one

To a stirred solution of 1-(2,4-dimethyl-5-nitro-phenyl)pyrrolidin-2-one (i.e. the product of Step A) (5 g, 21.3 mmol) in ethanol (40 mL) and water (12 mL) was added iron powder (6 g, 107 mmol) followed by ammonium chloride (1.13 g, 21.1 mmol). The mixture was stirred at 80° C. for 3 h then filtered through a pad of Celite® diatomaceous earth filter aid, rinsing with ethyl acetate (25 mL). The filtrate was concentrated under reduced pressure to give the title compound as an off white solid (4 g), which was used without further purification.

¹H NMR (CDCl₃) δ 6.92 (s, 1H), 6.46 (s, 1H), 3.67-3.64 (m, 2H), 3.53 (br s, 2H), 2.55-2.52 (m, 2H), 2.21-2.15 (m, 2H), 2.11 (s, 3H), 2.08 (s, 3H).

### Step C: Preparation of N-[2,4-dimethyl-5-(2-oxopyrrolidin-1-yl)phenyl]-1,1,1-trifluoro-methanesulfonamide

To a stirred solution of 1-(5-amino-2,4-dimethyl-phenyl)pyrrolidin-2-one (i.e. the product of Step B) (4 g, 19.6 mmol) in dichloromethane (40 mL) at −78° C. was added triethylamine (5.9 mL, 42 mmol) and trifluoromethanesulfonic anhydride (3.2 mL, 19 mmol). After 2 h, water (20 ml) was added and the mixture was extracted with ethyl acetate (200 mL×2). The combined organic layer was washed with brine (50 mL) and concentrated under reduced pressure. Column chromatography on silica gel gave the title compound as an off white solid (3 g).

¹H NMR (CDCl₃) δ 7.05 (s, 1H), 6.95 (s, 1H), 3.70-3.67 (m, 2H), 2.63-2.60 (m, 2H), 2.27-2.21 (m, 2H), 2.20 (s, 3H), 2.17 (s, 3H).

### Step D: Preparation of 1,1,1-trifluoro-N-[5-[3-(hydroxyimino)-2-oxo-1-pyrrolidinyl]-2,4-dimethylphenyl]methanesulfonamide

To a stirred solution of N-[2,4-dimethyl-5-(2-oxopyrrolidin-1-yl)phenyl]-1,1,1-trifluoro-methanesulfonamide (i.e. the product of Step C) (3 g, 8.9 mmol) in anhydrous tetrahydrofuran (30 mL) at 0° C. was added sodium bis(trimethylsilyl)amide (30 mL, 30 mmol, 1 M in tetrahydrofuran). The mixture was stirred at 0° C. for 30 min then isopentyl nitrite (2.2 g, 18.8 mmol) was added and the mixture was stirred at 0° C. for 2 h. The mixture was quenched with 1 N hydrochloric acid (30 mL) and extracted with ethyl acetate (100 mL×2). The combined organic layer was dried over sodium sulfate and concentrated under reduced pressure. Trituration with 10% diethyl ether/pentane gave the title compound as an off white solid (1.6 g).

¹H NMR (DMSO-d₆) δ 11.95 (s, 1H), 11.52 (br s, 1H), 7.24 (br s, 1H), 7.16 (s, 1H), 3.72 (m, 2H), 2.88 (m, 2H), 2.27 (s, 3H), 2.10 (s, 3H).

### Synthesis Example 5

### Preparation of N-[5-[3-(Ethoxyimino)-2-oxo-1-pyrrolidinyl]-2,4-dimethylphenyl]-1,1,1-trifluoromethanesulfonamide (Compound 12)

To a stirred solution of 1,1,1-Trifluoro-N-[5-[3-(hydroxyimino)-2-oxo-1-pyrrolidinyl]-2,4-dimethylphenyl]methanesulfonamide (i.e. the product of Step D in Synthesis Example 4) (0.4 g, 1.09 mmol) in tetrahydrofuran (20 mL) was added potassium tert-butoxide (3.8 ml, 3.8 mmol, 1 M in tetrahydrofuran) at room temperature. The mixture was stirred for 20 min then bromoethane (0.1 mL, 1.3 mmol) was added. After stirring for 16 h, the mixture was acidified to pH-4 with 1 N hydrochloric acid and extracted with ethyl acetate (50 mL×2). The combined organic layer was dried over sodium sulfate and concentrated under reduced pressure. Column chromatography on silica gel gave the title compound as an off white solid (160 mg).

¹H NMR (DMSO-d₆) δ 11.48 (br s, 1H), 7.26 (s, 1H), 7.19 (s, 1H), 4.24 (q, 2H), 3.73 (m, 2H), 2.90 (m, 2H), 2.28 (s, 3H), 2.11 (s, 3H), 1.27 (t, 3H).

By the procedures described herein together with methods known in the art, the following compounds of Tables 1 to 11 can be prepared. The following abbreviations are used in the Tables which follow: t means tertiary, s means secondary, n means normal, i means iso, c means cyclo, Me means methyl, Et means ethyl, Pr means propyl, Bu means butyl, i-Pr means isopropyl, Bu means butyl, c-Pr cyclopropyl, c-Bu means cyclobutyl, Ph means phenyl, OMe means methoxy, OEt means ethoxy, SMe means methylthio, SEt means ethylthio, NHMe means methylamino, —CN means cyano, Py means pyridinyl, —NC₂ means nitro, TMS means trimethylsilyl, S(O)Me means methylsulfinyl, and S(O)₂Me means methylsulfonyl.

TABLE 1

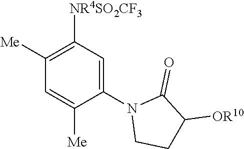

R⁴ = H

| R¹⁰ | R¹⁰ | R¹⁰ | R¹⁰ |
|---|---|---|---|
| CH₂CF₃ | CH₂CHF₂ | CH₂CH₂Cl | CH₂CH₂Br |
| CH₂CH═CH₂ | CH(Me)C═CH₂ | CH₂CH═CH(Me) | CH₂CH═C(Me)₂ |
| CH₂C(Cl)═CH₂ | CH₂CH₂CH═CH₂ | CH₂CO₂Me | CH₂(C═O)Me |
| propargyl | c-pro | c-butyl | c-pentyl |
| allyl | c-hexyl | CH₂CF═CH₂ | CH₂CH₂CN |
| J-1 | J-2 | J-3 | J-4 |
| J-5 | J-6 | J-7 | J-8 |
| J-9 | J-10 | J-11 | J-12 |
| J-13 | J-14 | J-15 | J-16 |
| J-17 | J-18 | J-19 | J-20 |
| J-21 | J-22 | | |

See Exhibit 2 for J-1 through J-22.

This disclosure also includes TABLES 2 through 25 wherein the Header Row Phrase in TABLE 1 (i.e. “R⁴═H”) is replaced with the Header Row Phrase listed in the respective TABLE, and the remaining variable(s) are as defined in TABLE 1.

| TABLE | Header Row Phrase |
|---|---|
| 2 | R⁴ = SO₂CF₃ |
| 3 | R⁴ = is SO₂CH₃ |
| 4 | R⁴ = COMe |
| 5 | R⁴ = COEt |
| 6 | R⁴ = CH₂OCO-t-Bu |
| 7 | R⁴ = CH₂OCO-n-Bu |
| 8 | R⁴ = CH₂OCO-sec-Bu |
| 9 | R⁴ = CH₂OCO-i-Bu |
| 10 | R⁴ = CH₂OCO-c-hexyl |
| 11 | R⁴ = CH₂OCO-c-pentyl |
| 12 | R⁴ = CH₂OCO-c-butyl |
| 13 | R⁴ = CH₂OCO-c-propyl |
| 14 | R⁴ CH₂OCOMe |
| 15 | R⁴ CH₂OCOCH₂CH₃ |
| 16 | R⁴ = CH₂OCOPh |
| 17 | R⁴ = CH₂OCO-n-Pr |
| 18 | R⁴ = CH₂OCO-i-Pr |
| 19 | R⁴ = (C═O)SMe |
| 20 | R⁴ = COOMe |
| 21 | R⁴ = COOEt |
| 22 | R⁴ = CO-n-Pr |
| 23 | R⁴ = CONMe2 |
| 24 | R⁴ = (C═O)N-morpholine |
| 25 | R4 = (C═S)N-morpholine |

TABLE 26

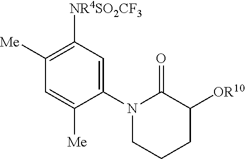

R⁴ = H

| R¹⁰ | R¹⁰ | R¹⁰ | R¹⁰ |
|---|---|---|---|
| CH₂CF₃ | CH₂CHF₂ | CH₂CH₂Cl | CH₂CH₂Br |
| CH₂CH═CH₂ | CH(Me)C═CH₂ | CH₂CH═CH(Me) | CH₂CH═C(Me)₂ |
| CH₂C(Cl)═CH₂ | CH₂CH₂CH═CH₂ | CH₂CO₂Me | CH₂(C═O)Me |
| propargyl | c-pro | c-butyl | c-pentyl |
| allyl | c-hexyl | CH₂CF═CH₂ | CH₂CH₂CN |
| J-1 | J-2 | J-3 | J-4 |
| J-5 | J-6 | J-7 | J-8 |
| J-9 | J-10 | J-11 | J-12 |
| J-13 | J-14 | J-15 | J-16 |
| J-17 | J-18 | J-19 | J-20 |
| J-21 | J-22 | | |

This disclosure also includes TABLES 27 through 50 wherein the Header Row Phrase in TABLE 26 (i.e. “R⁴═H”) is replaced with the Header Row Phrase listed in the respective TABLE, and the remaining variable(s) are as defined in TABLE 26.

| TABLE | Header Row Phrase |
|---|---|
| 27 | R⁴ = SC₂CF₃ |
| 28 | R⁴ = SO₂CH₃ |
| 29 | R⁴ = COMe |
| 30 | R⁴ = COEt |
| 31 | R⁴ = CH₂OCO-t-Bu |
| 32 | R⁴ = CH₂OCO-n-Bu |
| 33 | R⁴ = CH₂OCO-sec-Bu |
| 34 | R⁴ = CH₂OCO-i-Bu |
| 35 | R⁴ = CH₂OCO-c-hexyl |
| 36 | R⁴ = CH₂OCO-c-pentyl |
| 37 | R⁴ = CH₂OCO-c-butyl |
| 38 | R⁴ = CH₂OCO-c-propyl |
| 39 | R⁴ = CH₂OCOMe |
| 40 | R⁴ = CH₂OCOCH₂CH₃ |
| 41 | R⁴ = CH₂OCOPh |
| 42 | R⁴ = CH₂OCO-n-Pr |
| 43 | R⁴ = CH₂OCO-i-Pr |
| 44 | R⁴ = (C═O)SMe |
| 45 | R⁴ = COOMe |
| 46 | R⁴ = COOEt |
| 47 | R⁴ = CO-n-Pr |
| 48 | R⁴ = CONMe2 |
| 49 | R⁴ = (C═O)N-morpholine |
| 50 | R⁴ = (C═S)N-morpholine |

TABLE 51

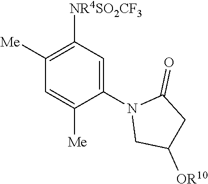

R⁴ = H

| R¹⁰ | R¹⁰ | R¹⁰ | R¹⁰ |
|---|---|---|---|
| CH₂CF₃ | CH₂CHF₂ | CH₂CH₂Cl | CH₂CH₂Br |
| CH₂CH═CH₂ | CH(Me)C═CH₂ | CH₂CH═CH(Me) | CH₂CH═C(Me)₂ |
| CH₂C(Cl)═CH₂ | CH₂CH₂CH═CH₂ | CH₂CO₂Me | CH₂(C═O)Me |
| propargyl | c-pro | c-butyl | c-pentyl |
| allyl | c-hexyl | CH₂CF═CH₂ | CH₂CH₂CN |
| J-1 | J-2 | J-3 | J-4 |
| J-5 | J-6 | J-7 | J-8 |
| J-9 | J-10 | J-11 | J-12 |
| J-13 | J-14 | J-15 | J-16 |
| J-17 | J-18 | J-19 | J-20 |
| J-21 | J-22 | | |

This disclosure also includes TABLES 52 through 75 wherein the Header Row Phrase in TABLE 51 (i.e. “R⁴ is H”) is replaced with the Header Row Phrase listed in the respective Table, and the R¹⁰ are as defined in TABLE 51.

| TABLE | Header Row Phrase |
|---|---|
| 52 | R⁴ = SO₂CF₃ |
| 53 | R⁴ is SO₂CH₃ |
| 54 | R⁴ = is COMe |
| 55 | R⁴ = COEt |
| 56 | R⁴ = CH₂OCO-t-Bu |
| 57 | R⁴ = CH₂OCO-n-Bu |
| 58 | R⁴ = CH₂OCO-sec-Bu |
| 59 | R⁴ = CH₂OCO-i-Bu |
| 60 | R⁴ = CH₂OCO-c-hexyl |
| 61 | R⁴ = CH₂OCO-c-pentyl |
| 62 | R⁴ = CH₂OCO-c-butyl |
| 63 | R⁴ = CH₂OCO-c-propyl |
| 64 | R⁴ = CH₂OCOMe |
| 65 | R⁴ = CH₂OCOCH₂CH₃ |
| 66 | R⁴ = CH₂OCOPh |
| 67 | R⁴ = CH₂OCO-n-Pr |
| 68 | R⁴ = CH₂OCO-i-Pr |
| 69 | R⁴ = (C═O)SMe |
| 70 | R⁴ = COOMe |
| 71 | R⁴ = COOEt |
| 72 | R⁴ = CO-n-Pr |
| 73 | R⁴ = CONMe2 |
| 74 | R⁴ = (C═O)N-morpholine |
| 75 | R⁴ = (C═S)N-morpholine |

TABLE 76

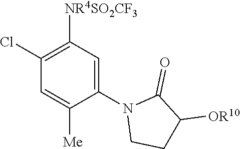

R⁴ = H

| R¹⁰ | R¹⁰ | R¹⁰ | R¹⁰ |
|---|---|---|---|
| CH₂CF₃ | CH₂CHF₂ | CH₂CH₂Cl | CH₂CH₂Br |
| CH₂CH═CH₂ | CH(Me)C═CH₂ | CH₂CH═CH(Me) | CH₂CH═C(Me)₂ |
| CH₂C(Cl)-CH₂ | CH₂CH₂CH═CH₂ | CH₂CO₂Me | CH₂(C═O)Me |
| propargyl | c-pro | c-butyl | c-pentyl |
| allyl | c-hexyl | CH₂CF═CH₂ | CH₂CH₂CN |
| J-1 | J-2 | J-3 | J-4 |
| J-5 | J-6 | J-7 | J-8 |
| J-9 | J-10 | J-11 | J-12 |
| J-13 | J-14 | J-15 | J-16 |
| J-17 | J-18 | J-19 | J-20 |
| J-21 | J-22 | | |

This disclosure also includes TABLES 77 through 100 wherein the Header Row Phrase in TABLE 76 (i.e. “R⁴═H”) is replaced with the Header Row Phrase listed in the respective TABLE, and the R¹⁰ are as defined in TABLE 76.

| TABLE | Header Row Phrase |
|---|---|
| 77 | R⁴ = SO₂CF₃ |
| 78 | R⁴ = SO₂CH₃ |
| 79 | R⁴ = COMe |
| 80 | R⁴ = COEt |
| 81 | R⁴ = CH₂OCO-t-Bu |
| 82 | R⁴ = CH₂OCO-n-Bu |
| 83 | R⁴ = CH₂OCO-sec-Bu |
| 84 | R⁴ = CH₂OCO-i-Bu |
| 85 | R⁴ = CH₂OCO-c-hexyl |
| 86 | R⁴ = CH₂OCO-c-pentyl |
| 87 | R⁴ = CH₂OCO-c-butyl |
| 88 | R⁴ = CH₂OCO-c-propyl |
| 89 | R⁴ CH₂OCOMe |
| 90 | R⁴ CH₂OCOCH₂CH₃ |
| 91 | R⁴ = CH₂OCOPh |
| 92 | R⁴ = CH₂OCO-n-Pr |
| 93 | R⁴ = CH₂OCO-i-Pr |
| 94 | R⁴ = (C═O)SMe |
| 95 | R⁴ = COOMe |
| 96 | R⁴ = COOEt |
| 97 | R⁴ = CO-n-Pr |
| 98 | R⁴ = CONMe2 |
| 99 | R⁴ = (C═O)N-morpholine |
| 100 | R⁴ = (C═S)N-morpholine |

TABLE 101

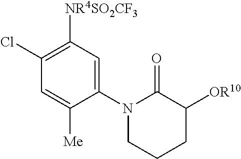

R⁴ = H

| R¹⁰ | R¹⁰ | R¹⁰ | R¹⁰ |
|---|---|---|---|
| CH₂CF₃ | CH₂CHF₂ | CH₂CH₂Cl | CH₂CH₂Br |
| CH₂CH═CH₂ | CH(Me)C═CH₂ | CH₂CH═CH(Me) | CH₂CH═C(Me)₂ |
| CH₂C(Cl)═CH₂ | CH₂CH₂CH═CH₂ | CH₂CO₂Me | CH₂(C-O)Me |
| propargyl | c-pro | c-butyl | c-pentyl |
| allyl | c-hexyl | CH₂CF═CH₂ | CH₂CH₂CN |
| J-1 | J-2 | J-3 | J-4 |
| J-5 | J-6 | J-7 | J-8 |
| J-9 | J-10 | J-11 | J-12 |
| J-13 | J-14 | J-15 | J-16 |
| J-17 | J-18 | J-19 | J-20 |
| J-21 | J-22 | | |

This disclosure also includes TABLES 102 through 125 wherein the Header Row Phrase in TABLE 101 (i.e. “R⁴═H”) is replaced with the Header Row Phrase listed in the respective TABLE, and the remaining variable(s) are as defined in TABLE 101.

| TABLE | Header Row Phrase |
|---|---|
| 102 | R⁴ = SO₂CF₃ |
| 103 | R⁴ = SO₂CH₃ |
| 104 | R⁴ = COMe |
| 105 | R⁴ = COEt |
| 106 | R⁴ = CH₂OCO-t-Bu |
| 107 | R⁴ = CH₂OCO-n-Bu |
| 108 | R⁴ = CH₂OCO-sec-Bu |
| 109 | R⁴ = CH₂OCO-i-Bu |
| 110 | R⁴ = CH₂OCO-c-hexyl |
| 111 | R⁴ = CH₂OCO-c-pentyl |
| 112 | R⁴ = CH₂OCO-c-butyl |
| 113 | R⁴ = CH₂OCO-c-propyl |
| 114 | R⁴ = CH₂OCOMe |
| 115 | R⁴ = CH₂OCOCH₂CH₃ |
| 116 | R⁴ = CH₂OCOPh |
| 117 | R⁴ = CH₂OCO-n-Pr |
| 118 | R⁴ = CH₂OCO-i-Pr |
| 119 | R⁴ = (C═O)SMe |
| 120 | R⁴ = COOMe |
| 121 | R⁴ = COOEt |
| 122 | R⁴ = CO-n-Pr |
| 123 | R⁴ = CONMe2 |
| 124 | R⁴ = (C═O)N-morpholine |
| 125 | R⁴ = (C═S)N-morpholine |

TABLE 126

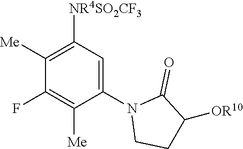

R⁴ = H

| R¹⁰ | R¹⁰ | R¹⁰ | R¹⁰ |
|---|---|---|---|
| CH₂CF₃ | CH₂CHF₂ | CH₂CH₂Cl | CH₂CH₂Br |
| CH₂CH═CH₂ | CH(Me)C═CH₂ | CH₂CH═CH(Me) | CH₂CH═C(Me)₂ |
| CH₂C(Cl)=CH₂ | CH₂CH₂CH═CH₂ | CH₂CO₂Me | CH₂(C═O)Me |
| propargyl | c-pro | c-butyl | c-pentyl |
| allyl | c-hexyl | CH₂CF=CH₂ | CH₂CH₂CN |
| J-1 | J-2 | J-3 | J-4 |
| J-5 | J-6 | J-7 | J-8 |
| J-9 | J-10 | J-11 | J-12 |
| J-13 | J-14 | J-15 | J-16 |
| J-17 | J-18 | J-19 | J-20 |
| J-21 | J-22 | | |

This disclosure also includes TABLES 127 through 150 wherein the Header Row Phrase in TABLE 126 (i.e. “R⁴═H”) is replaced with the Header Row Phrase listed in the respective TABLE, and the remaining variable(s) are as defined in TABLE 126.

| TABLE | Header Row Phrase |
|---|---|
| 127 | R⁴ = SO₂CF₃ |
| 128 | R⁴ = SO₂CH₃ |
| 129 | R⁴ = COMe |
| 130 | R⁴ = COEt |
| 131 | R⁴ = CH₂OCO-t-Bu |
| 132 | R⁴ = CH₂OCO-n-Bu |
| 133 | R⁴ = CH₂OCO-sec-Bu |
| 134 | R⁴ = CH₂OCO-i-Bu |
| 135 | R⁴ = CH₂OCO-c-hexyl |
| 136 | R⁴ = CH₂OCO-c-pentyl |
| 137 | R⁴ = CH₂OCO-c-butyl |
| 138 | R⁴ = CH₂OCO-c-propyl |
| 139 | R⁴ = CH₂OCOMe |
| 140 | R⁴ = CH₂OCOCH₂CH₃ |
| 141 | R⁴ = CH₂OCOPh |
| 142 | R⁴ = CH₂OCO-n-Pr |
| 143 | R⁴ = CH₂OCO-i-Pr |
| 144 | R⁴ = (C═O)SMe |
| 145 | R⁴ = COOMe |
| 146 | R⁴ = COOEt |
| 147 | R⁴ = CO-n-Pr |
| 148 | R⁴ = CONMe2 |
| 149 | R⁴ = (C═O)N-morpholine |
| 150 | R⁴ = (C═S)N-morpholine |

TABLE 151

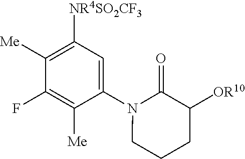

R⁴ = H

| R¹⁰ | R¹⁰ | R¹⁰ | R¹⁰ |
|---|---|---|---|
| CH₂CF₃ | CH₂CHF₂ | CH₂CH₂Cl | CH₂CH₂Br |
| CH₂CH═CH₂ | CH(Me)C═CH₂ | CH₂CH═CH(Me) | CH₂CH═C(Me)₂ |
| CH₂C(Cl)═CH₂ | CH₂CH₂CH═CH₂ | CH₂CO₂Me | CH₂(C═O)Me |
| propargyl | c-pro | c-butyl | c-pentyl |
| allyl | c-hexyl | CH₂CF=CH₂ | CH₂CH₂CN |
| J-1 | J-2 | J-3 | J-4 |
| J-5 | J-6 | J-7 | J-8 |
| J-9 | J-10 | J-11 | J-12 |
| J-13 | J-14 | J-15 | J-16 |
| J-17 | J-18 | J-19 | J-20 |
| J-21 | J-22 | | |

This disclosure also includes TABLES 152 through 175 wherein the Header Row Phrase in TABLE 151 (i.e. “R⁴ is H”) is replaced with the Header Row Phrase listed in the respective TABLE, and the remaining variable(s) are as defined in TABLE 151.

| TABLE | Header Row Phrase |
|---|---|
| 152 | R⁴ = SO₂CF₃ |
| 153 | R⁴ = SO₂CH₃ |
| 154 | R⁴ = COMe |
| 155 | R⁴ = COEt |
| 156 | R⁴ = CH₂OCO-t-Bu |
| 157 | R⁴ = CH₂OCO-n-Bu |
| 158 | R⁴ = CH₂OCO-sec-Bu |
| 159 | R⁴ = CH₂OCO-i-Bu |
| 160 | R⁴ = CH₂OCO-c-hexyl |
| 161 | R⁴ = CH₂OCO-c-pentyl |
| 162 | R⁴ = CH₂OCO-c-butyl |
| 163 | R⁴ = CH₂OCO-c-propyl |
| 164 | R⁴ = CH₂OCOMe |
| 165 | R⁴ = CH₂OCOCH₂CH₃ |
| 166 | R⁴ = CH₂OCOPh |
| 167 | R⁴ = CH₂OCO-n-Pr |
| 168 | R⁴ = CH₂OCO-i-Pr |
| 169 | R⁴ = (C═O)SMe |
| 170 | R⁴ = COOMe |
| 171 | R⁴ = COOEt |
| 172 | R⁴ = CO-n-Pr |
| 173 | R⁴ = CONMe2 |
| 174 | R⁴ = (C═O)N-morpholine |
| 175 | R⁴ = (C═S)N-morpholine |

TABLE 176

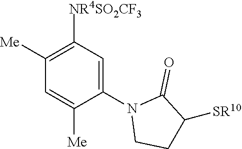

R⁴ = H

| R¹⁰ | R¹⁰ | R¹⁰ | R¹⁰ |
|---|---|---|---|
| CH₂CF₃ | CH₂CHF₂ | CH₂CH₂Cl | CH₂CH₂Br |
| CH₂CH═CH₂ | CH(Me)C═CH₂ | CH₂CH═CH(Me) | CH₂CH═C(Me)₂ |
| CH₂C(Cl)═CH₂ | CH₂CH₂CH═CH₂ | CH₂CO₂Me | CH₂(C═O)Me |
| propargyl | c-pro | c-butyl | c-pentyl |
| allyl | c-hexyl | CH₂CF=CH₂ | CH₂CH₂CN |
| J-1 | J-2 | J-3 | J-4 |
| J-5 | J-6 | J-7 | J-8 |
| J-9 | J-10 | J-11 | J-12 |
| J-13 | J-14 | J-15 | J-16 |
| J-17 | J-18 | J-19 | J-20 |
| J-21 | J-22 | | |

This disclosure also includes TABLES 177 through 200 wherein the Header Row Phrase in TABLE 176 (i.e. “R⁴═H”) is replaced with the Header Row Phrase listed in the respective TABLE, and the remaining variable(s) are as defined in TABLE 176.

| TABLE | Header Row Phrase |
|---|---|
| 177 | R⁴ = SO₂CF₃ |
| 178 | R⁴ = SO₂CH₃ |
| 179 | R⁴ = COMe |
| 180 | R⁴ = COEt |
| 181 | R⁴ = CH₂OCO-t-Bu |
| 182 | R⁴ = CH₂OCO-n-Bu |
| 183 | R⁴ = CH₂OCO-sec-Bu |
| 184 | R⁴ = CH₂OCO-i-Bu |
| 185 | R⁴ = CH₂OCO-c-hexyl |
| 186 | R⁴ = CH₂OCO-c-pentyl |
| 187 | R⁴ = CH₂OCO-c-butyl |
| 188 | R⁴ = CH₂OCO-c-propyl |
| 189 | R⁴ = CH₂OCOMe |
| 190 | R⁴ = CH₂OCOCH₂CH₃ |
| 191 | R⁴ = CH₂OCOPh |
| 192 | R⁴ = CH₂OCO-n-Pr |
| 193 | R⁴ = CH₂OCO-i-Pr |
| 194 | R⁴ = (C═O)SMe |
| 195 | R⁴ = COOMe |
| 196 | R⁴ = COOEt |
| 197 | R⁴ = CO-n-Pr |
| 198 | R⁴ = CONMe2 |
| 199 | R⁴ = (C═O)N-morpholine |
| 200 | R⁴ = (C═S)N-morpholine |

TABLE 201

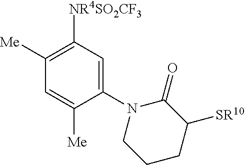

R⁴ = H

| R¹⁰ | R¹⁰ | R¹⁰ | R¹⁰ |
|---|---|---|---|
| CH₂CF₃ | CH₂CHF₂ | CH₂CH₂Cl | CH₂CH₂Br |
| CH₂CH═CH₂ | CH(Me)C═CH₂ | CH₂CH═CH(Me) | CH₂CH═C(Me)₂ |
| CH₂C(Cl)═CH₂ | CH₂CH₂CH═CH₂ | CH₂CO₂Me | CH₂(C═O)Me |
| propargyl | c-pro | c-butyl | c-pentyl |
| allyl | c-hexyl | CH₂CF=CH₂ | CH₂CH₂CN |
| J-1 | J-2 | J-3 | J-4 |
| J-5 | J-6 | J-7 | J-8 |
| J-9 | J-10 | J-11 | J-12 |
| J-13 | J-14 | J-15 | J-16 |
| J-17 | J-18 | J-19 | J-20 |
| J-21 | J-22 | | |

This disclosure also includes TABLES 202 through 225 wherein the Header Row Phrase in TABLE 201 (i.e. “R⁴═H”) is replaced with the Header Row Phrase listed in the respective TABLE, and the remaining variable(s) are as defined in TABLE 201.

| TABLE | Header Row Phrase |
|---|---|
| 202 | R⁴ = SO₂CF₃ |
| 203 | R⁴ = SO₂CH₃ |
| 204 | R⁴ = COMe |
| 205 | R⁴ = COEt |
| 206 | R⁴ = CH₂OCO-t-Bu |
| 207 | R⁴ = CH₂OCO-n-Bu |
| 208 | R⁴ = CH₂OCO-sec-Bu |
| 209 | R⁴ = CH₂OCO-i-Bu |
| 210 | R⁴ = CH₂OCO-c-hexyl |
| 211 | R⁴ = CH₂OCO-c-pentyl |
| 212 | R⁴ = CH₂OCO-c-butyl |
| 213 | R⁴ = CH₂OCO-c-propyl |
| 214 | R⁴ = CH₂OCOMe |
| 215 | R⁴ = CH₂OCOCH₂CH₃ |
| 216 | R⁴ = CH₂OCOPh |
| 217 | R⁴ = CH₂OCO-n-Pr |
| 218 | R⁴ = CH₂OCO-i-Pr |
| 219 | R⁴ = (C═O)SMe |
| 220 | R⁴ = COOMe |
| 221 | R⁴ = COOEt |
| 222 | R⁴ = CO-n-Pr |
| 223 | R⁴ = CONMe2 |
| 224 | R⁴ = (C═O)N-morpholine |
| 225 | R⁴ = (C═S)N-morpholine |

TABLE 226

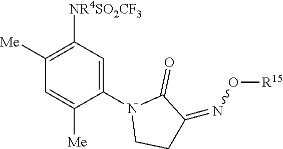

| | |
|---|---|
| 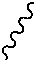 | indicates the bond can be cis or trans |

R⁴ = H

| R¹⁵ | R¹⁵ | R¹⁵ | R¹⁵ |
|---|---|---|---|
| H | Me | Et | n-Pr |
| i-Pr | n-Bu | i-Bu | CH₂F |
| CF₂H | CH₂CF₃ | CH₂CF₂H | allyl |
| propargyl | CH₂c-Pr (J-5) | CH₂c-Bu (J-6) | |

This disclosure also includes TABLES 227 through 250 wherein the Header Row Phrase in TABLE 226 (i.e. “R⁴═H”) is replaced with the Header Row Phrase listed in the respective TABLE, and the remaining variable(s) are as defined in TABLE 226.

| TABLE | Header Row Phrase |
|---|---|
| 227 | R⁴ = SO₂CF₃ |
| 228 | R⁴ = SO₂CH₃ |
| 229 | R⁴ = COMe |
| 230 | R⁴ = COEt |
| 231 | R⁴ = CH₂OCO-t-Bu |
| 232 | R⁴ = CH₂OCO-n-Bu |
| 233 | R⁴ = CH₂OCO-sec-Bu |
| 234 | R⁴ = CH₂OCO-i-Bu |
| 235 | R⁴ = CH₂OCO-c-hexyl |
| 236 | R⁴ = CH₂OCO-c-pentyl |
| 237 | R⁴ = CH₂OCO-c-butyl |
| 238 | R⁴ = CH₂OCO-c-propyl |
| 239 | R⁴ CH₂OCOMe |
| 240 | R⁴ CH₂OCOCH₂CH₃ |
| 241 | R⁴ = CH₂OCOPh |
| 242 | R⁴ = CH₂OCO-n-Pr |
| 243 | R⁴ = CH₂OCO-i-Pr |
| 244 | R⁴ = (C═O)SMe |
| 245 | R⁴ = COOMe |
| 246 | R⁴ = COOEt |
| 247 | R⁴ = CO-n-Pr |
| 248 | R⁴ = CONMe2 |
| 249 | R⁴ = (C═O)N-morpholine |
| 250 | R⁴ = (C═S)N-morpholine |

TABLE 251

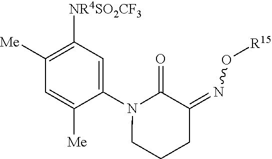

| | |
|---|---|
| 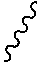 | indicates the bond can be cis or trans |

R⁴ = H

| R¹⁵ | R¹⁵ | R¹⁵ | R¹⁵ |
|---|---|---|---|
| H | Me | Et | n-Pr |
| i-Pr | n-Bu | i-Bu | CH₂F |
| CF₂H | CH₂CF₃ | CH₂CF₂H | allyl |
| propargyl | CH₂c-Pr (J-5) | CH₂c-Bu (J-6) | |

This disclosure also includes TABLES 252 through 275 wherein the Header Row Phrase in TABLE 251 (i.e. “R⁴═H”) is replaced with the Header Row Phrase listed in the respective TABLE, and the remaining variable(s) are as defined in TABLE 251.

| TABLE | Header Row Phrase |
|---|---|
| 252 | R⁴ = SO₂CF₃ |
| 253 | R⁴ = SO₂CH₃ |
| 254 | R⁴ = COMe |
| 255 | R⁴ = COEt |
| 256 | R⁴ = CH₂OCO-t-Bu |
| 257 | R⁴ = CH₂OCO-n-Bu |
| 258 | R⁴ = CH₂OCO-sec-Bu |
| 259 | R⁴ = CH₂OCO-i-Bu |
| 260 | R⁴ = CH₂OCO-c-hexyl |
| 261 | R⁴ = CH₂OCO-c-pentyl |
| 262 | R⁴ = CH₂OCO-c-butyl |
| 263 | R⁴ = CH₂OCO-c-propyl |
| 264 | R⁴ CH₂OCOMe |
| 265 | R⁴ CH₂OCOCH₂CH₃ |
| 266 | R⁴ = CH₂OCOPh |
| 267 | R⁴ = CH₂OCO-n-Pr |
| 268 | R⁴ = CH₂OCO-i-Pr |
| 269 | R⁴ = (C═O)SMe |
| 270 | R⁴ = COOMe |
| 271 | R⁴ = COOEt |
| 272 | R⁴ = CO-n-Pr |
| 273 | R⁴ = CONMe2 |
| 274 | R⁴ = (C═O)N-morpholine |
| 275 | R⁴ = (C═S)N-morpholine |

TABLE 276

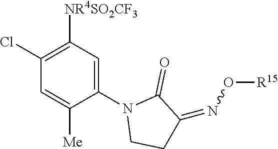

| | |
|---|---|
| 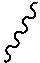 | indicates the bond can be cis or trans |

R⁴ = H

| R¹⁵ | R¹⁵ | R¹⁵ | R¹⁵ |
|---|---|---|---|
| H | Me | Et | n-Pr |
| i-Pr | n-Bu | i-Bu | CH₂F |
| CF₂H | CH₂CF₃ | CH₂CF₂H | allyl |
| propargyl | CH₂c-Pr (J-5) | CH₂c-Bu (J-6) | |

This disclosure also includes TABLES 277 through 300 wherein the Header Row Phrase in TABLE 276 (i.e. “R⁴═H”) is replaced with the Header Row Phrase listed in the respective TABLE, and the remaining variable(s) are as defined in TABLE 276.

| TABLE | Header Row Phrase |
|---|---|
| 277 | R⁴ = SO₂CF₃ |
| 278 | R⁴ = SO₂CH₃ |
| 279 | R⁴ = COMe |
| 280 | R⁴ = COEt |
| 281 | R⁴ = CH₂OCO-t-Bu |
| 282 | R⁴ = CH₂OCO-n-Bu |
| 283 | R⁴ = CH₂OCO-sec-Bu |
| 284 | R⁴ = CH₂OCO-i-Bu |
| 285 | R⁴ = CH₂OCO-c-hexyl |
| 286 | R⁴ = CH₂OCO-c-pentyl |
| 287 | R⁴ = CH₂OCO-c-butyl |
| 288 | R⁴ = CHOCO-c-propyl |
| 289 | R⁴ CHOCOMe |
| 290 | R⁴ CH₂OCOCH₂CH₃ |
| 291 | R⁴ = CH₂OCOPh |
| 292 | R⁴ = CH₂OCO-n-Pr |
| 293 | R⁴ = CH₂OCO-i-Pr |
| 294 | R⁴ = (C═O)SMe |
| 295 | R⁴ = COOMe |
| 296 | R⁴ = COOEt |
| 297 | R⁴ = CO-n-Pr |
| 298 | R⁴ = CONMe2 |
| 299 | R⁴ = (C═O)N-morpholine |
| 300 | R⁴ = (C═S)N-morpholine |

TABLE 301

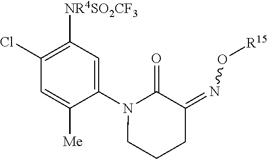

| | |
|---|---|
| 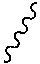 | indicates the bond can be cis or trans |

R⁴ = H

| R¹⁵ | R¹⁵ | R¹⁵ | R¹⁵ |
|---|---|---|---|
| H | Me | Et | n-Pr |
| i-Pr | n-Bu | i-Bu | CH₂F |
| CF₂H | CH₂CF₃ | CH₂CF₂H | allyl |
| propargyl | CH₂c-Pr (J-5) | CH₂c-Bu (J-6) | |

This disclosure also includes TABLES 302 through 325 wherein the Header Row Phrase in TABLE 301 (i.e. “R⁴═H”) is replaced with the Header Row Phrase listed in the respective TABLE, and the remaining variable(s) are as defined in TABLE 301.

| TABLE | Header Row Phrase |
|---|---|
| 302 | R⁴ = SO₂CF₃ |
| 303 | R⁴ = SO₂CH₃ |
| 304 | R⁴ = COMe |
| 305 | R⁴ = COEt |
| 306 | R⁴ = CH₂OCO-t-Bu |
| 307 | R⁴ = CH₂OCO-n-Bu |
| 308 | R⁴ = CH₂OCO-sec-Bu |
| 309 | R⁴ = CH₂OCO-i-Bu |
| 310 | R⁴ = CH₂OCO-c-hexyl |
| 311 | R⁴ = CH₂OCO-c-pentyl |
| 312 | R⁴ = CH₂OCO-c-butyl |
| 313 | R⁴ = CH₂OCO-c-propyl |
| 314 | R⁴ CH₂OCOMe |
| 315 | R⁴ CH₂OCOCH₂CH₃ |
| 316 | R⁴ = CH₂OCOPh |
| 317 | R⁴ = CH₂OCO-n-Pr |
| 318 | R⁴ = CH₂OCO-i-Pr |
| 319 | R⁴ = (C═O)SMe |
| 320 | R⁴ = COOMe |
| 321 | R⁴ = COOEt |
| 322 | R⁴ = CO-n-Pr |
| 323 | R⁴ = CONMe² |
| 324 | R⁴ = (C═O)N-morpholine |
| 325 | R⁴ = (C═S)N-morpholine |

TABLE 326

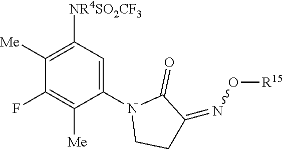

| | |
|---|---|
| 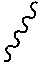 | indicates the bond can be cis or trans |

R⁴ = H

| R¹⁵ | R¹⁵ | R¹⁵ | R¹⁵ |
|---|---|---|---|
| H | Me | Et | n-Pr |
| i-Pr | n-Bu | i-Bu | CH₂F |
| CF₂H | CH₂CF₃ | CH₂CF₂H | allyl |
| propargyl | CH₂c-Pr (J-5) | CH₂c-Bu (J-6) | |

This disclosure also includes TABLES 327 through 350 wherein the Header Row Phrase in TABLE 326 (i.e. “R⁴═H”) is replaced with the Header Row Phrase listed in the respective TABLE, and the remaining variable(s) are as defined in TABLE 326.

| TABLE | Header Row Phrase |
|---|---|
| 327 | R⁴ = SO₂CF₃ |
| 328 | R⁴ = SO₂CH₃ |
| 329 | R⁴ = COMe |
| 330 | R⁴ = COEt |
| 331 | R⁴ = CH₂OCO-t-Bu |
| 332 | R⁴ = CH₂OCO-n-Bu |
| 333 | R⁴ = CH₂OCO-sec-Bu |
| 334 | R⁴ = CH₂OCO-i-Bu |
| 335 | R⁴ = CH₂OCO-c-hexyl |
| 336 | R⁴ = CH₂OCO-c-pentyl |
| 337 | R⁴ = CH₂OCO-c-butyl |
| 338 | R⁴ = CH₂OCO-c-propyl |
| 339 | R⁴ CH₂OCOMe |
| 340 | R⁴ CH₂OCOCH₂CH₃ |
| 341 | R⁴ = CH₂OCOPh |
| 342 | R⁴ = CH₂OCO-n-Pr |
| 343 | R⁴ = CH₂OCO-i-Pr |
| 344 | R⁴ = (C═O)SMe |
| 345 | R⁴ = COOMe |
| 346 | R⁴ = COOEt |
| 347 | R⁴ = CO-n-Pr |
| 348 | R⁴ = CONMe2 |
| 349 | R⁴ = (C═O)N-morpholine |
| 350 | R⁴ = (C═S)N-morpholine |

TABLE 351

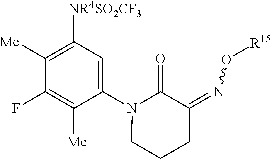

| | |
|---|---|
| 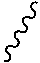 | indicates the bond can be cis or trans |

R⁴ = H

| R¹⁵ | R¹⁵ | R¹⁵ | R¹⁵ |
|---|---|---|---|
| H | Me | Et | n-Pr |
| i-Pr | n-Bu | i-Bu | CH₂F |
| CF₂H | CH₂CF₃ | CH₂CF₂H | allyl |
| propargyl | CH₂c-Pr (J-5) | CH₂c-Bu (J-6) | |

This disclosure also includes TABLES 352 through 375 wherein the Header Row Phrase in TABLE 351 (i.e. “R⁴═H”) is replaced with the Header Row Phrase listed in the respective TABLE, and the remaining variable(s) are as defined in TABLE 351.

| TABLE | Header Row Phrase |
|---|---|
| 352 | R⁴ = SO₂CF₃ |
| 353 | R⁴ = SO₂CH₃ |
| 354 | R⁴ = COMe |
| 355 | R⁴ = COEt |
| 356 | R⁴ = CH₂OCO-t-Bu |
| 357 | R⁴ = CH₂OCO-n-Bu |
| 358 | R⁴ = CH₂OCO-sec-Bu |
| 359 | R⁴ = CH₂OCO-i-Bu |
| 360 | R⁴ = CH₂OCO-c-hexyl |
| 361 | R⁴ = CH₂OCO-c-pentyl |
| 362 | R⁴ = CH₂OCO-c-butyl |
| 363 | R⁴ = CH₂OCO-c-propyl |
| 364 | R⁴ CH₂OCOMe |
| 365 | R⁴ CH₂OCOCH₂CH₃ |
| 366 | R⁴ = CH₂OCOPh |
| 367 | R⁴ = CH₂OCO-n-Pr |
| 368 | R⁴ = CH₂OCO-i-Pr |
| 369 | R⁴ = (C═O)SMe |
| 370 | R⁴ = COOMe |
| 371 | R⁴ = COOEt |
| 372 | R⁴ = CO-n-Pr |
| 373 | R⁴ = CONMe2 |
| 374 | R⁴ = (C═O)N-morpholine |
| 375 | R⁴ = (C═S)N-morpholine |

### Formulation/Utility

A compound of this disclosure will generally be used as a herbicidal active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil-in-water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil-in-water emulsion, flowable concentrate and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about from about one to several thousand liters per hectare, but more typically are in the range from about ten to several hundred liters per hectare. Sprayable formulations can be tank mixed with water or another suitable medium for foliar treatment by aerial or ground application or for application to the growing medium of the plant. Liquid and dry formulations can be metered directly into drip irrigation systems or metered into the furrow during planting.

The formulations will typically contain effective amounts of active ingredient, diluent and surfactant within the following approximate ranges which add up to 100 percent by weight.

### Weight Percent

| | Active | | |
|---|---|---|---|
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions (including | | | |
| Emulsifiable Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, New Jersey.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quatemary salts and diquatemary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all percentages are by weight and all formulations are prepared in conventional ways. Compound numbers refer to compounds in Index Table A Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

### Example A

### High Strength Concentrate

| | |
|---|---|
| Compound 1 | 98.5% |
| silica aerogel | 0.5% |
| synthetic amorphous fine silica | 1.0% |

### Example B

### Wettable Powder

| | |
|---|---|
| Compound 1 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

### Granule

| | |
|---|---|
| Compound 1 | 10.0% |
| attapulgite granules (low volatile matter, 0.71/0.30 mm; | 90.0% |
| U.S.S. No. 25-50 sieves) | |

### Example D

### Extruded Pellet

| | |
|---|---|
| Compound 1 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

### Emulsifiable Concentrate

| | |
|---|---|
| Compound 1 | 10.0% |
| polyoxyethylene sorbitol hexoleate | 20.0% |
| C₆-C₁₀ fatty acid methyl ester | 70.0% |

### Example F

### Microemulsion

| | |
|---|---|
| Compound 1 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

### Suspension Concentrate

| | |
|---|---|
| Compound 1 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example H

### Emulsion in Water

| | |
|---|---|
| Compound 1 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example I

### Oil Dispersion

| | |
|---|---|
| Compound 1 | 25% |
| polyoxyethylene sorbitol hexaoleate | 15% |
| organically modified bentonite clay | 2.5% |
| fatty acid methyl ester | 57.5% |

Additional Example Formulations include Examples A through I above wherein “Compound 1” is replaced in each of the Examples A through I with the respective compounds from Index Table A as shown below.

| Compound No. | Compound No. | Compound No. | Compound No. | Compound No. |
|---|---|---|---|---|
| Compound 2 | Compound 4 | Compound 10 | Compound 11 | Compound 18 |
| Compound 3 | Compound 5 | Compound 12 | Compound 13 | Compound 19 |
| Compound 6 | Compound 7 | Compound 14 | Compound 15 | Compound 20 |
| Compound 8 | Compound 9 | Compound 16 | Compound 17 | Compound 21 |
| Compound No. | Compound No. | Compound No. | Compound No. | Compound No. |
| Compound 22 | Compound 24 | Compound 30 | Compound 31 | Compound 38 |
| Compound 23 | Compound 25 | Compound 32 | Compound 33 | Compound 39 |
| Compound 26 | Compound 27 | Compound 34 | Compound 35 | Compound 40 |
| Compound 28 | Compound 29 | Compound 36 | Compound 37 | Compound 41 |
| Compound 42 | Compound 44 | Compound 46 | Compound 47 | Compound 50 |
| Compound 43 | Compound 45 | Compound 48 | Compound 49 | Compound 51 |
| Compound 52 | Compound 53 | Compound 54 | Compound 55 | Compound 56 |
| Compound 57 | Compound 58 | Compound 59 | Compound 60 | Compound 61 |
| Compound 62 | Compound 63 | Compound 64 | | |

Test results indicate that the compounds of the present invention are highly active preemergent and/or postemergent herbicides and/or plant growth regulants. The compounds of the disclosure generally show highest activity for postemergence weed control (i.e. applied after weed seedlings emerge from the soil) and preemergence weed control (i.e. applied before weed seedlings emerge from the soil). Many of them have utility for broad-spectrum pre- and/or postemergence weed control in areas where complete control of all vegetation is desired such as around fuel storage tanks, industrial storage areas, parking lots, drive-in theaters, air fields, river banks, irrigation and other waterways, around billboards and highway and railroad structures. Many of the compounds of this invention, by virtue of selective metabolism in crops versus weeds or by selective activity at the locus of physiological inhibition in crops and weeds or by selective placement on or within the environment of a mixture of crops and weeds, are useful for the selective control of grass and broadleaf weeds within a crop/weed mixture. One skilled in the art will recognize that the preferred combination of these selectivity factors within a compound or group of compounds can readily be determined by performing routine biological and/or biochemical assays. Compounds of this invention may show tolerance to important agronomic crops including, but is not limited to, alfalfa, barley, cotton, wheat, rape, sugar beets, corn (maize), sorghum, soybeans, rice, oats, peanuts, vegetables, tomato, potato, perennial plantation crops including coffee, cocoa, oil palm, rubber, sugarcane, citrus, grapes, fruit trees, nut trees, banana, plantain, pineapple, hops, tea and forests such as eucalyptus and conifers (e.g., loblolly pine), and turf species (e.g., Kentucky bluegrass, St. Augustine grass, Kentucky fescue and Bermuda grass). Compounds of this invention can be used in crops genetically transformed or bred to incorporate resistance to herbicides, express proteins toxic to invertebrate pests (such as Bacillus thuringiensis toxin), and/or express other useful traits. Those skilled in the art will appreciate that not all compounds are equally effective against all weeds. Alternatively, the subject compounds are useful to modify plant growth.

As the compounds of the invention have both preemergent and postemergent herbicidal activity, to control undesired vegetation by killing or injuring the vegetation or reducing its growth, the compounds can be usefully applied by a variety of methods involving contacting a herbicidally effective amount of a compound of the disclosure or a composition comprising said compound and at least one of a surfactant, a solid diluent or a liquid diluent, to the foliage or other part of the undesired vegetation or to the environment of the undesired vegetation such as the soil or water in which the undesired vegetation is growing or which surrounds the seed or other propagule of the undesired vegetation. Undesired vegetation includes at least one selected from the group consisting of grass weeds and broadleaf weeds. Undesired vegetation is selected from the group consisting of annual bluegrass, Benghal dayflower, blackgrass, black nightshade, broadleaf signalgrass, Canada thistle, cheat, common cocklebur (Xanthium pensylvanicum), common ragweed, corn poppies, field violet, giant foxtail, goosegrass, green foxtail, guinea grass, hairy beggarticks, herbicide-resistant black grass, horseweed, Italian rye grass, jimsonweed, Johnson grass (Sorghum halepense), large crabgrass, little seed canary grass, morning glory, Pennsylvania smartweed, pitted morning glory, prickly sida, quackgrass, redroot pigweed, shattercane, shepherd's purse, silky windgrass, sunflower (as weed in potato), wild buckwheat (Polygonum convolvulus), wild mustard (Brassica kaber), wild oat (Avena fatua), wild pointsettia, yellow foxtail, and yellow nutsedge (Cyperus esculentus).

A herbicidally effective amount of the compounds of this invention is determined by a number of factors. These factors include: formulation selected, method of application, amount and type of vegetation present, growing conditions, etc. In general, a herbicidally effective amount of compounds of this invention is about 0.001 to 20 kg/ha with a preferred range of about 0.004 to 1 kg/ha. One skilled in the art can easily determine the herbicidally effective amount necessary for the desired level of weed control.

In one common embodiment, a compound of the disclosure is applied, typically in a formulated composition, to a locus comprising desired vegetation (e.g., crops) and undesired vegetation (i.e. weeds), both of which may be seeds, seedlings and/or larger plants, in contact with a growth medium (e.g., soil). In this locus, a composition comprising a compound of the disclosure can be directly applied to a plant or a part thereof, particularly of the undesired vegetation, and/or to the growth medium in contact with the plant.

Plant varieties and cultivars of the desired vegetation in the locus treated with a compound of the disclosure can be obtained by conventional propagation and breeding methods or by genetic engineering methods. Genetically modified plants (transgenic plants) are those in which a heterologous gene (transgene) has been stably integrated into the plant's genome. A transgene that is defined by its particular location in the plant genome is called a transformation or transgenic event.

Genetically modified plant cultivars in the locus which can be treated according to the invention include those that are resistant against one or more biotic stresses (pests such as nematodes, insects, mites, fungi, etc.) or abiotic stresses (drought, cold temperature, soil salinity, etc.) or that contain other desirable characteristics. Plants can be genetically modified to exhibit traits of, for example, herbicide tolerance, insect-resistance, modified oil profiles or drought tolerance.

Although most typically, compounds of the invention are used to control undesired vegetation, contact of desired vegetation in the treated locus with compounds of the invention may result in super-additive or synergistic effects with genetic traits in the desired vegetation, including traits incorporated through genetic modification. For example, resistance to phytophagous insect pests or plant diseases, tolerance to biotic/abiotic stresses or storage stability may be greater than expected from the genetic traits in the desired vegetation.

Compounds of this invention can also be mixed with one or more other biologically active compounds or agents including herbicides, herbicide safeners, fungicides, insecticides, nematocides, bactericides, acaricides, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Mixtures of the compounds of the invention with other herbicides can broaden the spectrum of activity against additional weed species, and suppress the proliferation of any resistant biotypes. Thus the present invention also pertains to a composition comprising a compound of Formula 1 (in a herbicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix or one or more other biologically active compounds or agents can be formulated separately from the compound of Formula 1, and the formulations combined together before application (e.g., in a spray tank) or, alternatively, applied in succession.

A mixture of one or more of the following herbicides with a compound of this invention may be particularly useful for weed control: acetochlor, acifluorfen and its sodium salt, aclonifen, acrolein (2-propenal), alachlor, alloxydim, ametryn, amicarbazone, amidosulfuron, aminocyclopyrachlor and its esters (e.g., methyl, ethyl) and salts (e.g., sodium, potassium), aminopyralid, amitrole, ammonium sulfamate, anilofos, asulam, atrazine, azimsulfuron, beflubutamid, beflubutamid-M, benazolin, benazolin-ethyl, bencarbazone, benfluralin, benfuresate, bensulfuron-methyl, bensulide, bentazone, benzobicyclon, benzofenap, bicyclopyrone, bifenox, bilanafos, bispyribac and its sodium salt, bixlozone, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinmethylin, cinosulfuron, clacyfos, clefoxydim, clethodim, clodinafop-propargyl, clomazone, clomeprop, clopyralid, clopyralid-olamine, cloransulam-methyl, cumyluron, cyanazine, cycloate, cyclopyrimorate, cyclosulfamuron, cycloxydim, cyhalofop-butyl, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimesulfazet, dimethachlor, dimethametryn, dimethenamid, dimethenamid-P, dimethipin, dimethylarsinic acid and its sodium salt, dinitramine, dinoterb, diphenamid, diquat dibromide, dithiopyr, diuron, DNOC, endothal, EPTC, epyrifenacil, esprocarb, ethalfluralin, ethametsulfuron-methyl, ethiozin, ethofumesate, ethoxyfen, ethoxysulfuron, etobenzanid, fenoxaprop-ethyl, fenoxaprop-P-ethyl, fenoxasulfone, fenquinotrione, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, fluazolate, flucarbazone, flucetosulfuron, fluchloralin, flufenacet, flufenpyr, flufenpyr-ethyl, flumetsulam, flumiclorac-pentyl, flumioxazin, fluometuron, fluoroglycofen-ethyl, flupoxam, flupyrsulfuron-methyl and its sodium salt, flurenol, flurenol-butyl, fluridone, flurochloridone, fluroxypyr, flurtamone, fluthiacet-methyl, fomesafen, foramsulfuron, fosamine-ammonium, glufosinate, glufosinate-ammonium, glufosinate-P, glyphosate and its salts such as ammonium, isopropylammonium, potassium, sodium (including sesquisodium) and trimesium (alternatively named sulfosate), halauxifen, halauxifen-methyl, halosulfuron-methyl, haloxyfop-etotyl, haloxyfop-methyl, hexazinone, hydantocidin, imazamethabenz-methyl, imazamox, imazapic, imazapyr, imazaquin, imazaquin-ammonium, imazethapyr, imazethapyr-ammonium, imazosulfuron, indanofan, indaziflam, iofensulfuron, iodosulfuron-methyl, ioxynil, ioxynil octanoate, ioxynil-sodium, ipfencarbazone, isoproturon, isouron, isoxaben, isoxaflutole, isoxachlortole, lactofen, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, metazosulfuron, methabenzthiazuron, methylarsonic acid and its calcium, monoammonium, monosodium and disodium salts, methyldymron, metobenzuron, metobromuron, metolachlor, S-metolachlor, metosulam, metoxuron, metribuzin, metsulfuron-methyl, molinate, monolinuron, naproanilide, napropamide, napropamide-M, naptalam, neburon, nicosulfuron, norflurazon orbencarb or thosulfamuron oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxamid, pethoxyamid, phenmedipham, picloram, picloram-potassium, picolinafen, pinoxaden, piperophos, pretilachlor, primisulfuron-methyl, prodiamine, profoxydim, prometon, prometryn, propachlor, propanil, propaquizafop, propazine, propham, propisochlor, propoxycarbazone, propyrisulfuron, propyzamide, prosulfocarb, prosulfuron, pyraclonil, pyraflufen-ethyl, pyrasulfotole, pyrazogyl, pyrazolynate, pyrazoxyfen, pyrazosulfuron-ethyl, pyribenzoxim, pyributicarb, pyridate, pyriftalid, pyriminobac-methyl, pyrimisulfan, pyrithiobac, pyrithiobac-sodium, pyroxasulfone, pyroxsulam, quinclorac, quinmerac, quinoclamine, quizalofop-ethyl, quizalofop-P-ethyl, quizalofop-P-tefuryl, rimsulfuron, saflufenacil, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tefuryltrione, tembotrione, tepraloxydim, terbacil, terbumeton, terbuthylazine, terbutryn, tetflupyrolimet, thenylchlor, thiazopyr, thiencarbazone, thifensulfuron-methyl, thiobencarb, tiafenacil, tiocarbazil, tolpyralate, topramezone, tralkoxydim, tri-allate, triafamone, triasulfuron, triaziflam, tribenuron-methyl, triclopyr, triclopyr-butotyl, triclopyr-triethylammonium, tridiphane, trietazine, trifloxysulfuron, trifludimoxazin, trifluralin, triflusulfuron-methyl, tritosulfuron, vernolate, 3-(2-chloro-3,6-difluorophenyl)-4-hydroxy-1-methyl-1,5-naphthyridin-2(1H)-one, 5-chloro-3-[(2-hydroxy-6-oxo-1-cyclohexen-1-yl)carbonyl]-1-(4-methoxyphenyl)-2(1H)-quinoxalinone, 2-chloro-N-(1-methyl-1H-tetrazol-5-yl)-6-(trifluoromethyl)-3-pyridinecarboxamide, 7-(3,5-dichloro-4-pyridinyl)-5-(2,2-difluoroethyl)-8-hydroxypyrido[2,3-b]pyrazin-6(5H)-one), 4-(2,6-diethyl-4-methylphenyl)-5-hydroxy-2,6-dimethyl-3(2H)-pyridazinone), 5-[[(2,6-difluorophenyl)methoxy]methyl]-4,5-dihydro-5-methyl-3-(3-methyl-2-thienyl)isoxazole (previously methioxolin), 4-(4-fluorophenyl)-6-[(2-hydroxy-6-oxo-1-cyclohexen-1-yl)carbonyl]-2-methyl-1,2,4-triazine-3,5(2H,4H)-dione, methyl 4-amino-3-chloro-6-(4-chloro-2-fluoro-3-methoxyphenyl)-5-fluoro-2-pyridinecarboxylate, 2-methyl-3-(methylsulfonyl)-N-(1-methyl-1H-tetrazol-5-yl)-4-(trifluoromethyl)benzamide and 2-methyl-N-(4-methyl-1,2,5-oxadiazol-3-yl)-3-(methylsulfinyl)-4-(trifluoromethyl)benzamide. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butl.) Butl. and Puccinia thlaspeos Schub.

Compounds of this invention can also be used in combination with plant growth regulators such as aviglycine, N-(phenylmethyl)-1H-purin-6-amine, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

General references for agricultural protectants (i.e. herbicides, herbicide safeners, insecticides, fungicides, nematocides, acaricides and biological agents) include The Pesticide Manual, 13th Edition, C. D. S. Tomlin, Ed., British Crop Protection Council, Farnham, Surrey, U. K., 2003 and The BioPesticide Manual, 2nd Edition, L. G. Copping, Ed., British Crop Protection Council, Famham, Surrey, U. K., 2001.

For embodiments where one or more of these various mixing partners are used, the mixing partners are typically used in the amounts similar to amounts customary when the mixture partners are used alone. More particularly in mixtures, active ingredients are often applied at an application rate between one-half and the full application rate specified on product labels for use of active ingredient alone. These amounts are listed in references such as The Pesticide Manual and The BioPesticide Manual. The weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of weeds controlled beyond the spectrum controlled by the compound of Formula 1 alone.

In certain instances, combinations of a compound of this invention with other biologically active (particularly herbicidal) compounds or agents (i.e. active ingredients) can result in a greater-than-additive (i.e. synergistic) effect on weeds and/or a less-than-additive effect (i.e. safening) on crops or other desirable plants. Reducing the quantity of active ingredients released in the environment while ensuring effective pest control is always desirable. Ability to use greater amounts of active ingredients to provide more effective weed control without excessive crop injury is also desirable. When synergism of herbicidal active ingredients occurs on weeds at application rates giving agronomically satisfactory levels of weed control, such combinations can be advantageous for reducing crop production cost and decreasing environmental load. When safening of herbicidal active ingredients occurs on crops, such combinations can be advantageous for increasing crop protection by reducing weed competition.

Of note is a combination of a compound of the disclosure with at least one other herbicidal active ingredient. Of particular note is such a combination where the other herbicidal active ingredient has different site of action from the compound of the invention. In certain instances, a combination with at least one other herbicidal active ingredient having a similar spectrum of control but a different site of action will be particularly advantageous for resistance management. Thus, a composition of the present invention can further comprise (in a herbicidally effective amount) at least one additional herbicidal active ingredient having a similar spectrum of control but a different site of action.

Compounds of this invention can also be used in combination with herbicide safeners such as allidochlor, benoxacor, cloquintocet-mexyl, cumyluron, cyometrinil, cyprosulfonamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone naphthalic anhydride (1,8-naphthalic anhydride), oxabetrinil, N-(aminocarbonyl)-2-methylbenzenesulfonamide, N-(aminocarbonyl)-2-fluorobenzenesulfonamide, 1-bromo-4-[(chloromethyl)sulfonyl]benzene (BCS), 4-(dichloroacetyl)-1-oxa-4-azospiro[4.5]decane (MON 4660), 2-(dichloromethyl)-2-methyl-1,3-dioxolane (MG 191), ethyl 1,6-dihydro-1-(2-methoxyphenyl)-6-oxo-2-phenyl-5-pyrimidinecarboxylate, 2-hydroxy-N,N-dimethyl-6-(trifluoromethyl)pyridine-3-carboxamide, and 3-oxo-1-cyclohexen-1-yl 1-(3,4-dimethylphenyl)-1,6-dihydro-6-oxo-2-phenyl-5-pyrimidinecarboxylate, 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide to increase safety to certain crops. Antidotally effective amounts of the herbicide safeners can be applied at the same time as the compounds of this invention or applied as seed treatments. Therefore an aspect of the present invention relates to a herbicidal mixture comprising a compound of this invention and an antidotally effective amount of a herbicide safener. Seed treatment is particularly useful for selective weed control, because it physically restricts antidoting to the crop plants. Therefore a particularly useful embodiment of the present invention is a method for selectively controlling the growth of undesired vegetation in a crop comprising contacting the locus of the crop with a herbicidally effective amount of a compound of this invention wherein seed from which the crop is grown is treated with an antidotally effective amount of safener. Antidotally effective amounts of safeners can be easily determined by one skilled in the art through simple experimentation.

Compounds of the invention cans also be mixed with: (1) polynucleotides including but not limited to DNA, RNA, and/or chemically modified nucleotides influencing the amount of a particular target through down regulation, interference, suppression or silencing of the genetically derived transcript that render a herbicidal effect; or (2) polynucleotides including but not limited to DNA, RNA, and/or chemically modified nucleotides influencing the amount of a particular target through down regulation, interference, suppression or silencing of the genetically derived transcript that render a safening effect.

Of note is a composition comprising a compound of the disclosure (in a herbicidally effective amount), at least one additional active ingredient selected from the group consisting of other herbicides and herbicide safeners (in an effective amount), and at least one component selected from the group consisting of surfactants, solid diluents and liquid diluents.

Preferred for better control of undesired vegetation (e.g., lower use rate such as from synergism, broader spectrum of weeds controlled or enhanced crop safety) or for preventing the development of resistant weeds are mixtures of a compound of this invention with a herbicide selected from the group consisting of atrazine, azimsulfuron, beflubutamid, S-beflubutamid, benzisothiazolinone, carfentrazone-ethyl, chlorimuron-ethyl, chlorsulfuron-methyl, clomazone, clopyralid potassium, cloransulam-methyl, 2-[(2,4-dichlorophenyl)methyl]-4,4-dimethyl-3-isoxazolidinone (CA No. 81777-95-9) and 2-[(2,5-dichlorophenyl)methyl]-4,4-dimethyl-3-isoxazolidinone (CA No. 81778-66-7) ethametsulfuron-methyl, flumetsulam, 4-(4-fluorophenyl)-6-[(2-hydroxy-6-oxo-1-cyclohexen-1-yl)carbonyl]-2-methyl-1,2,4-triazine-3,5-(2H,4H)-dione, flupyrsulfuron-methyl, fluthiacet-methyl, fomesafen, imazethapyr, lenacil, mesotrione, metribuzin, metsulfuron-methyl, pethoxamid, picloram, pyroxasulfone, quinclorac, rimsulfuron, rinskor, S-metolachlor, sulfentrazone, thifensulfuron-methyl, triflusulfuron-methyl and tribenuron-methyl.

Table A1 lists specific combinations of a Component (a) with Component (b) illustrative of the mixtures, compositions and methods of the present invention. Compound # in the Component (a) column is identified in Index Table A. The second column of Table A1 lists the specific Component (b) compound (e.g., “2,4-D” in the first line). The third, fourth and fifth columns of Table A1 lists ranges of weight ratios for rates at which the Component (a) compound is typically applied to a field-grown crop relative to Component (b) (i.e. (a):(b)). Thus, for example, the first line of Table A1 specifically discloses the combination of Component (a) (i.e. Compound 45 in Index Table A) with 2,4-D is typically applied in a weight ratio between 1:192-6:1. The remaining lines of Table A1 are to be construed similarly.

TABLE A1

| Component (a) | | Typical | More Typical | Most Typical |
|---|---|---|---|---|
| (Compound #) | Component (b) | Weight Ratio | Weight Ratio | Weight Ratio |
| 1 | 2,4-D | 1:192-6:1 | 1:64-2:1 | 1:24-1:3 |
| 1 | Acetochlor | 1:768-2:1 | 1:256-1:2 | 1:96-1:11 |
| 1 | Acifluorfen | 1:96-12:1 | 1:32-4:1 | 1:12-1:2 |
| 1 | Aclonifen | 1:857-2:1 | 1:285-1:3 | 1:107-1:12 |
| 1 | Alachlor | 1:768-2:1 | 1:256-1:2 | 1:96-1:11 |
| 1 | Ametryn | 1:384-3:1 | 1:128-1:1 | 1:48-1:6 |
| 1 | Amicarbazone | 1:192-6:1 | 1:64-2:1 | 1:24-1:3 |
| 1 | Amidosulfuron | 1:6-168:1 | 1:2-56:1 | 1:1-11:1 |
| 1 | Aminocyclopyrachlor | 1:48-24:1 | 1:16-8:1 | 1:6-2:1 |
| 1 | Aminopyralid | 1:20-56:1 | 1:6-19:1 | 1:2-4:1 |
| 1 | Amitrole | 1:768-2:1 | 1:256-1:2 | 1:96-1:11 |
| 1 | Anilofos | 1:96-12:1 | 1:32-4:1 | 1:12-1:2 |
| 1 | Asulam | 1:960-2:1 | 1:320-1:3 | 1:120-1:14 |
| 1 | Atrazine | 1:192-6:1 | 1:64-2:1 | 1:24-1:3 |
| 1 | Azimsulfuron | 1:6-168:1 | 1:2-56:1 | 1:1-11:1 |
| 1 | Beflubutamid | 1:342-4:1 | 1:114-2:1 | 1:42-1:5 |
| 1 | Benfuresate | 1:617-2:1 | 1:205-1:2 | 1:77-1:9 |
| 1 | Bensulfuron-methyl | 1:25-45:1 | 1:8-15:1 | 1:3-3:1 |
| 1 | Bentazone | 1:192-6:1 | 1:64-2:1 | 1:24-1:3 |
| 1 | Benzobicyclon | 1:85-14:1 | 1:28-5:1 | 1:10-1:2 |
| 1 | Benzofenap | 1:257-5:1 | 1:85-2:1 | 1:32-1:4 |
| 1 | Bicyclopyrone | 1:42-27:1 | 1:14-9:1 | 1:5-2:1 |
| 1 | Bifenox | 1:257-5:1 | 1:85-2:1 | 1:32-1:4 |
| 1 | Bispyribac-sodium | 1:10-112:1 | 1:3-38:1 | 1:1-7:1 |
| 1 | Bromacil | 1:384-3:1 | 1:128-1:1 | 1:48-1:6 |
| 1 | Bromobutide | 1:384-3:1 | 1:128-1:1 | 1:48-1:6 |
| 1 | Bromoxynil | 1:96-12:1 | 1:32-4:1 | 1:12-1:2 |
| 1 | Butachlor | 1:768-2:1 | 1:256-1:2 | 1:96-1:11 |
| 1 | Butafenacil | 1:42-27:1 | 1:14-9:1 | 1:5-2:1 |
| 1 | Butylate | 1:1542-1:2 | 1:514-1:5 | 1:192-1:22 |
| 1 | Carfenstrole | 1:192-6:1 | 1:64-2:1 | 1:24-1:3 |
| 1 | Carfentrazone-ethyl | 1:128-9:1 | 1:42-3:1 | 1:16-1:2 |
| 1 | Chlorimuron-ethyl | 1:8-135:1 | 1:2-45:1 | 1:1-9:1 |
| 1 | Chlorotoluron | 1:768-2:1 | 1:256-1:2 | 1:96-1:11 |
| 1 | Chlorsulfuron | 1:6-168:1 | 1:2-56:1 | 1:1-11:1 |
| 1 | Cincosulfuron | 1:17-68:1 | 1:5-23:1 | 1:2-5:1 |
| 1 | Cinidon-ethyl | 1:384-3:1 | 1:128-1:1 | 1:48-1:6 |
| 1 | Cinmethylin | 1:34-34:1 | 1:11-12:1 | 1:4-3:1 |
| 1 | Clacyfos | 1:34-34:1 | 1:11-12:1 | 1:4-3:1 |
| 1 | Clethodim | 1:48-24:1 | 1:16-8:1 | 1:6-2:1 |
| 1 | Clodinafop-propargyl | 1:20-56:1 | 1:6-19:1 | 1:2-4:1 |
| 1 | Clomazone | 1:384-3:1 | 1:128-1:1 | 1:48-1:6 |
| 1 | Clomeprop | 1:171-7:1 | 1:57-3:1 | 1:21-1:3 |
| 1 | Clopyralid | 1:192-6:1 | 1:64-2:1 | 1:24-1:3 |
| 1 | Cloransulam-methyl | 1:12-96:1 | 1:4-32:1 | 1:1-6:1 |
| 1 | Cumyluron | 1:384-3:1 | 1:128-1:1 | 1:48-1:6 |
| 1 | Cyanazine | 1:384-3:1 | 1:128-1:1 | 1:48-1:6 |
| 1 | Cyclopyrimorate | 1:17-68:1 | 1:5-23:1 | 1:2-5:1 |
| 1 | Cyclosulfamuron | 1:17-68:1 | 1:5-23:1 | 1:2-5:1 |
| 1 | Cycloxydim | 1:96-12:1 | 1:32-4:1 | 1:12-1:2 |
| 1 | Cyhalofop | 1:25-45:1 | 1:8-15:1 | 1:3-3:1 |
| 1 | Daimuron | 1:192-6:1 | 1:64-2:1 | 1:24-1:3 |
| 1 | Desmedipham | 1:322-4:1 | 1:107-2:1 | 1:40-1:5 |
| 1 | Dicamba | 1:192-6:1 | 1:64-2:1 | 1:24-1:3 |
| 1 | Dichlobenil | 1:1371-1:2 | 1:457-1:4 | 1:171-1:20 |
| 1 | Dichlorprop | 1:925-2:1 | 1:308-1:3 | 1:115-1:13 |
| 1 | Diclofop-methyl | 1:384-3:1 | 1:128-1:1 | 1:48-1:6 |
| 1 | Diclosulam | 1:10-112:1 | 1:3-38:1 | 1:1-7:1 |
| 1 | Difenzoquat | 1:288-4:1 | 1:96-2:1 | 1:36-1:4 |
| 1 | Diflufenican | 1:857-2:1 | 1:285-1:3 | 1:107-1:12 |
| 1 | Diflufenzopyr | 1:12-96:1 | 1:4-32:1 | 1:1-6:1 |
| 1 | Dimethachlor | 1:768-2:1 | 1:256-1:2 | 1:96-1:11 |
| 1 | Dimethametryn | 1:192-6:1 | 1:64-2:1 | 1:24-1:3 |
| 1 | Dimethenamid-P | 1:384-3:1 | 1:128-1:1 | 1:48-1:6 |
| 1 | Dithiopyr | 1:192-6:1 | 1:64-2:1 | 1:24-1:3 |
| 1 | Diuron | 1:384-3:1 | 1:128-1:1 | 1:48-1:6 |
| 1 | EPTC | 1:768-2:1 | 1:256-1:2 | 1:96-1:11 |
| 1 | Esprocarb | 1:1371-1:2 | 1:457-1:4 | 1:171-1:20 |
| 1 | Ethalfluralin | 1:384-3:1 | 1:128-1:1 | 1:48-1:6 |
| 1 | Ethametsulfuron-methyl | 1:17-68:1 | 1:5-23:1 | 1:2-5:1 |
| 1 | Ethoxyfen | 1:8-135:1 | 1:2-45:1 | 1:1-9:1 |
| 1 | Ethoxysulfuron | 1:20-56:1 | 1:6-19:1 | 1:2-4:1 |
| 1 | Etobenzanid | 1:257-5:1 | 1:85-2:1 | 1:32-1:4 |
| 1 | Fenoxaprop-ethyl | 1:120-10:1 | 1:40-4:1 | 1:15-1:2 |
| 1 | Fenoxasulfone | 1:85-14:1 | 1:28-5:1 | 1:10-1:2 |
| 1 | Fenquinotrione | 1:17-68:1 | 1:5-23:1 | 1:2-5:1 |
| 1 | Fentrazamide | 1:17-68:1 | 1:5-23:1 | 1:2-5:1 |
| 1 | Flazasulfuron | 1:17-68:1 | 1:5-23:1 | 1:2-5:1 |
| 1 | Florasulam | 1:2-420:1 | 1:1-140:1 | 2:1-27:1 |
| 1 | Fluazifop-butyl | 1:192-6:1 | 1:64-2:1 | 1:24-1:3 |
| 1 | Flucarbazone | 1:8-135:1 | 1:2-45:1 | 1:1-9:1 |
| 1 | Flucetosulfuron | 1:8-135:1 | 1:2-45:1 | 1:1-9:1 |
| 1 | Flufenacet | 1:257-5:1 | 1:85-2:1 | 1:32-1:4 |
| 1 | Flumetsulam | 1:24-48:1 | 1:8-16:1 | 1:3-3:1 |
| 1 | Flumiclorac-pentyl | 1:10-112:1 | 1:3-38:1 | 1:1-7:1 |
| 1 | Flumioxazin | 1:25-45:1 | 1:8-15:1 | 1:3-3:1 |
| 1 | Fluometuron | 1:384-3:1 | 1:128-1:1 | 1:48-1:6 |
| 1 | Flupyrsulfuron-methyl | 1:3-336:1 | 1:1-112:1 | 2:1-21:1 |
| 1 | Fluridone | 1:384-3:1 | 1:128-1:1 | 1:48-1:6 |
| 1 | Fluroxypyr | 1:96-12:1 | 1:32-4:1 | 1:12-1:2 |
| 1 | Flurtamone | 1:857-2:1 | 1:285-1:3 | 1:107-1:12 |
| 1 | Fluthiacet-methyl | 1:48-42:1 | 1:16-14:1 | 1:3-3:1 |
| 1 | Fomesafen | 1:96-12:1 | 1:32-4:1 | 1:12-1:2 |
| 1 | Foramsulfuron | 1:13-84:1 | 1:4-28:1 | 1:1-6:1 |
| 1 | Glufosinate | 1:288-4:1 | 1:96-2:1 | 1:36-1:4 |
| 1 | Glyphosate | 1:288-4:1 | 1:96-2:1 | 1:36-1:4 |
| 1 | Halosulfuron-methyl | 1:17-68:1 | 1:5-23:1 | 1:2-5:1 |
| 1 | Halauxifen | 1:20-56:1 | 1:6-19:1 | 1:2-4:1 |
| 1 | Halauxifen methyl | 1:20-56:1 | 1:6-19:1 | 1:2-4:1 |
| 1 | Haloxyfop-methyl | 1:34-34:1 | 1:11-12:1 | 1:4-3:1 |
| 1 | Hexazinone | 1:192-6:1 | 1:64-2:1 | 1:24-1:3 |
| 1 | Hydantocidin | 1:1100-16:1 | 1:385-8:1 | 1:144-4:1 |
| 1 | Imazamox | 1:13-84:1 | 1:4-28:1 | 1:1-6:1 |
| 1 | Imazapic | 1:20-56:1 | 1:6-19:1 | 1:2-4:1 |
| 1 | Imazapyr | 1:85-14:1 | 1:28-5:1 | 1:10-1:2 |
| 1 | Imazaquin | 1:34-34:1 | 1:11-12:1 | 1:4-3:1 |
| 1 | Imazethabenz-methyl | 1:171-7:1 | 1:57-3:1 | 1:21-1:3 |
| 1 | Imazethapyr | 1:24-48:1 | 1:8-16:1 | 1:3-3:1 |
| 1 | Imazosulfuron | 1:27-42:1 | 1:9-14:1 | 1:3-3:1 |
| 1 | Indanofan | 1:342-4:1 | 1:114-2:1 | 1:42-1:5 |
| 1 | Indaziflam | 1:25-45:1 | 1:8-15:1 | 1:3-3:1 |
| 1 | Iodosulfuron-methyl | 1:3-336:1 | 1:1-112:1 | 2:1-21:1 |
| 1 | Ioxynil | 1:192-6:1 | 1:64-2:1 | 1:24-1:3 |
| 1 | Ipfencarbazone | 1:85-14:1 | 1:28-5:1 | 1:10-1:2 |
| 1 | Isoproturon | 1:384-3:1 | 1:128-1:1 | 1:48-1:6 |
| 1 | Isoxaben | 1:288-4:1 | 1:96-2:1 | 1:36-1:4 |
| 1 | Isoxaflutole | 1:60-20:1 | 1:20-7:1 | 1:7-2:1 |
| 1 | Lactofen | 1:42-27:1 | 1:14-9:1 | 1:5-2:1 |
| 1 | Lenacil | 1:384-3:1 | 1:128-1:1 | 1:48-1:6 |
| 1 | Linuron | 1:384-3:1 | 1:128-1:1 | 1:48-1:6 |
| 1 | MCPA | 1:192-6:1 | 1:64-2:1 | 1:24-1:3 |
| 1 | MCPB | 1:288-4:1 | 1:96-2:1 | 1:36-1:4 |
| 1 | Mecoprop | 1:768-2:1 | 1:256-1:2 | 1:96-1:11 |
| 1 | Mefenacet | 1:384-3:1 | 1:128-1:1 | 1:48-1:6 |
| 1 | Mefluidide | 1:192-6:1 | 1:64-2:1 | 1:24-1:3 |
| 1 | Mesosulfuron-methyl | 1:5-224:1 | 1:1-75:1 | 1:1-14:1 |
| 1 | Mesotrione | 1:42-27:1 | 1:14-9:1 | 1:5-2:1 |
| 1 | Metamifop | 1:42-27:1 | 1:14-9:1 | 1:5-2:1 |
| 1 | Metazachlor | 1:384-3:1 | 1:128-1:1 | 1:48-1:6 |
| 1 | Metazosulfuron | 1:25-45:1 | 1:8-15:1 | 1:3-3:1 |
| 1 | Methabenzthiazuron | 1:768-2:1 | 1:256-1:2 | 1:96-1:11 |
| 1 | Metolachlor | 1:768-2:1 | 1:256-1:2 | 1:96-1:11 |
| 1 | Metosulam | 1:8-135:1 | 1:2-45:1 | 1:1-9:1 |
| 1 | Metribuzin | 1:192-6:1 | 1:64-2:1 | 1:24-1:3 |
| 1 | Metsulfuron-methyl | 1:2-560:1 | 1:1-187:1 | 3:1-35:1 |
| 1 | Molinate | 1:1028-2:1 | 1:342-1:3 | 1:128-1:15 |
| 1 | Napropamide | 1:384-3:1 | 1:128-1:1 | 1:48-1:6 |
| 1 | Napropamide-M | 1:192-6:1 | 1:64-2:1 | 1:24-1:3 |
| 1 | Naptalam | 1:192-6:1 | 1:64-2:1 | 1:24-1:3 |
| 1 | Nicosulfuron | 1:12-96:1 | 1:4-32:1 | 1:1-6:1 |
| 1 | Norflurazon | 1:1152-1:1 | 1:384-1:3 | 1:144-1:16 |
| 1 | Orbencarb | 1:1371-1:2 | 1:457-1:4 | 1:171-1:20 |
| 1 | Orthosulfamuron | 1:20-56:1 | 1:6-19:1 | 1:2-4:1 |
| 1 | Oryzalin | 1:514-3:1 | 1:171-1:2 | 1:64-1:8 |
| 1 | Oxadiargyl | 1:384-3:1 | 1:128-1:1 | 1:48-1:6 |
| 1 | Oxadiazon | 1:548-3:1 | 1:182-1:2 | 1:68-1:8 |
| 1 | Oxasulfuron | 1:27-42:1 | 1:9-14:1 | 1:3-3:1 |
| 1 | Oxaziclomefone | 1:42-27:1 | 1:14-9:1 | 1:5-2:1 |
| 1 | Oxyfluorfen | 1:384-3:1 | 1:128-1:1 | 1:48-1:6 |
| 1 | Paraquat | 1:192-6:1 | 1:64-2:1 | 1:24-1:3 |
| 1 | Pendimethalin | 1:384-3:1 | 1:128-1:1 | 1:48-1:6 |
| 1 | Penoxsulam | 1:10-112:1 | 1:3-38:1 | 1:1-7:1 |
| 1 | Penthoxamid | 1:384-3:1 | 1:128-1:1 | 1:48-1:6 |
| 1 | Pentoxazone | 1:102-12:1 | 1:34-4:1 | 1:12-1:2 |
| 1 | Phenmedipham | 1:102-12:1 | 1:34-4:1 | 1:12-1:2 |
| 1 | Picloram | 1:96-12:1 | 1:32-4:1 | 1:12-1:2 |
| 1 | Picolinafen | 1:34-34:1 | 1:11-12:1 | 1:4-3:1 |
| 1 | Pinoxaden | 1:25-45:1 | 1:8-15:1 | 1:3-3:1 |
| 1 | Pretilachlor | 1:192-6:1 | 1:64-2:1 | 1:24-1:3 |
| 1 | Primisulfuron-methyl | 1:8-135:1 | 1:2-45:1 | 1:1-9:1 |
| 1 | Prodiamine | 1:384-3:1 | 1:128-1:1 | 1:48-1:6 |
| 1 | Profoxydim | 1:42-27:1 | 1:14-9:1 | 1:5-2:1 |
| 1 | Prometryn | 1:384-3:1 | 1:128-1:1 | 1:48-1:6 |
| 1 | Propachlor | 1:1152-1:1 | 1:384-1:3 | 1:144-1:16 |
| 1 | Propanil | 1:384-3:1 | 1:128-1:1 | 1:48-1:6 |
| 1 | Propaquizafop | 1:48-24:1 | 1:16-8:1 | 1:6-2:1 |
| 1 | Propoxycarbazone | 1:17-68:1 | 1:5-23:1 | 1:2-5:1 |
| 1 | Propyrisulfuron | 1:17-68:1 | 1:5-23:1 | 1:2-5:1 |
| 1 | Propyzamide | 1:384-3:1 | 1:128-1:1 | 1:48-1:6 |
| 1 | Prosulfocarb | 1:1200-1:2 | 1:400-1:4 | 1:150-1:17 |
| 1 | Prosulfuron | 1:6-168:1 | 1:2-56:1 | 1:1-11:1 |
| 1 | Pyraclonil | 1:42-27:1 | 1:14-9:1 | 1:5-2:1 |
| 1 | Pyraflufen-ethyl | 1:5-224:1 | 1:1-75:1 | 1:1-14:1 |
| 1 | Pyrasulfotole | 1:13-84:1 | 1:4-28:1 | 1:1-6:1 |
| 1 | Pyrazolynate | 1:857-2:1 | 1:285-1:3 | 1:107-1:12 |
| 1 | Pyrazosulfuron-ethyl | 1:10-112:1 | 1:3-38:1 | 1:1-7:1 |
| 1 | Pyrazoxyfen | 1:5-224:1 | 1:1-75:1 | 1:1-14:1 |
| 1 | Pyribenzoxim | 1:10-112:1 | 1:3-38:1 | 1:1-7:1 |
| 1 | Pyributicarb | 1:384-3:1 | 1:128-1:1 | 1:48-1:6 |
| 1 | Pyridate | 1:288-4:1 | 1:96-2:1 | 1:36-1:4 |
| 1 | Pyriftalid | 1:10-112:1 | 1:3-38:1 | 1:1-7:1 |
| 1 | Pyriminobac-methyl | 1:20-56:1 | 1:6-19:1 | 1:2-4:1 |
| 1 | Pyrimisulfan | 1:17-68:1 | 1:5-23:1 | 1:2-5:1 |
| 1 | Pyrithiobac | 1:24-48:1 | 1:8-16:1 | 1:3-3:1 |
| 1 | Pyroxasulfone | 1:85-14:1 | 1:28-5:1 | 1:10-1:2 |
| 1 | Pyroxsulam | 1:5-224:1 | 1:1-75:1 | 1:1-14:1 |
| 1 | Quinclorac | 1:192-6:1 | 1:64-2:1 | 1:24-1:3 |
| 1 | Quizalofop-ethyl | 1:42-27:1 | 1:14-9:1 | 1:5-2:1 |
| 1 | Rimsulfuron | 1:13-84:1 | 1:4-28:1 | 1:1-6:1 |
| 1 | Saflufenacil | 1:25-45:1 | 1:8-15:1 | 1:3-3:1 |
| 1 | Sethoxydim | 1:96-12:1 | 1:32-4:1 | 1:12-1:2 |
| 1 | Simazine | 1:384-3:1 | 1:128-1:1 | 1:48-1:6 |
| 1 | Sulcotrione | 1:120-10:1 | 1:40-4:1 | 1:15-1:2 |
| 1 | Sulfentrazone | 1:147-8:1 | 1:49-3:1 | 1:18-1:3 |
| 1 | Sulfometuron-methyl | 1:34-34:1 | 1:11-12:1 | 1:4-3:1 |
| 1 | Sulfosulfuron | 1:8-135:1 | 1:2-45:1 | 1:1-9:1 |
| 1 | Tebuthiuron | 1:384-3:1 | 1:128-1:1 | 1:48-1:6 |
| 1 | Tefuryltrione | 1:42-27:1 | 1:14-9:1 | 1:5-2:1 |
| 1 | Tembotrione | 1:31-37:1 | 1:10-13:1 | 1:3-3:1 |
| 1 | Tepraloxydim | 1:25-45:1 | 1:8-15:1 | 1:3-3:1 |
| 1 | Terbacil | 1:288-4:1 | 1:96-2:1 | 1:36-1:4 |
| 1 | Terbuthylazine | 1:857-2:1 | 1:285-1:3 | 1:107-1:12 |
| 1 | Terbutryn | 1:192-6:1 | 1:64-2:1 | 1:24-1:3 |
| 1 | Thenylchlor | 1:85-14:1 | 1:28-5:1 | 1:10-1:2 |
| 1 | Thiazopyr | 1:384-3:1 | 1:128-1:1 | 1:48-1:6 |
| 1 | Thiencarbazone | 1:3-336:1 | 1:1-112:1 | 2:1-21:1 |
| 1 | Thifensulfuron-methyl | 1:5-224:1 | 1:1-75:1 | 1:1 - 14:1 |
| 1 | Tiafenacil | 1:17-68:1 | 1:5-23:1 | 1:2- 5:1 |
| 1 | Thiobencarb | 1:768-2:1 | 1:256-1:2 | 1:96 - 1:11 |
| 1 | Tolpyralate | 1:31-37:1 | 1:10-13:1 | 1:3- 3:1 |
| 1 | Topramzone | 1:6-168:1 | 1:2-56:1 | 1:1- 11:1 |
| 1 | Tralkoxydim | 1:68-17:1 | 1:22-6:1 | 1:8- 2:1 |
| 1 | Triafamone | 1:2-420:1 | 1:1-140:1 | 2:1- 27:1 |
| 1 | Triallate | 1:768-2:1 | 1:256-1:2 | 1:96 - 1:11 |
| 1 | Triasulfuron | 1:5-224:1 | 1:1-75:1 | 1:1 - 14:1 |
| 1 | Triaziflam | 1:171-7:1 | 1:57-3:1 | 1:21 - 1:3 |
| 1 | Tribenuron-methyl | 1:3-336:1 | 1:1-112:1 | 2:1- 21:1 |
| 1 | Triclopyr | 1:192-6:1 | 1:64-2:1 | 1:24 - 1:3 |
| 1 | Trifloxysulfuron | 1:2-420:1 | 1:1-140:1 | 2:1- 27:1 |
| 1 | Trifludimoxazin | 1:25-45:1 | 1:8-15:1 | 1:3- 3:1 |
| 1 | Trifluralin | 1:288-4:1 | 1:96-2:1 | 1:36 - 1:4 |
| 1 | Triflusulfuron-methyl | 1:17-68:1 | 1:5-23:1 | 1:2 - 5:1 |
| 1 | Tritosulfuron | 1:13-84:1 | 1:4-28:1 | 1:1-6:1 |

Table A2 is constructed the same as Table A1 above except that entries below the “Component (a)” column heading are replaced with the respective Component (a) Column Entry shown below. Compound No. in the Component (a) column is identified in Index Table A. Thus, for example, in Table A2 the entries below the “Component (a)” column heading all recite “Compound 2” (i.e. Compound 2 identified in Index Table A), and the first line below the column headings in Table A2 specifically discloses a mixture of Compound 2 with 2,4-D. Tables A3 through A64 are constructed similarly.

| Table Number | Component (a) Column Entries |
|---|---|
| A2 | 2 |
| A3 | 3 |
| A4 | 4 |
| A5 | 5 |
| A6 | 6 |
| A7 | 7 |
| A8 | 8 |
| A9 | 9 |
| A10 | 10 |
| A11 | 11 |
| A12 | 12 |
| A13 | 13 |
| A14 | 14 |
| A15 | 15 |
| A16 | 16 |
| A17 | 17 |
| A18 | 18 |
| A19 | 19 |
| A20 | 20 |
| A21 | 21 |
| A22 | 22 |
| A23 | 23 |
| A24 | 24 |
| A25 | 25 |
| A26 | 26 |
| A27 | 27 |
| A28 | 28 |
| A29 | 29 |
| A30 | 30 |
| A31 | 31 |
| A32 | 32 |
| A33 | 33 |
| A34 | 34 |
| A35 | 35 |
| A36 | 36 |
| A37 | 37 |
| A38 | 38 |
| A39 | 39 |
| A40 | 40 |
| A41 | 41 |
| A42 | 42 |
| A43 | 43 |
| A44 | 44 |
| A45 | 45 |
| A46 | 46 |
| A47 | 47 |
| A48 | 48 |
| A49 | 49 |
| A50 | 50 |
| A51 | 51 |
| A52 | 52 |
| A53 | 53 |
| A54 | 54 |
| A55 | 55 |
| A56 | 56 |
| A57 | 57 |
| A58 | 58 |
| A59 | 59 |
| A60 | 60 |
| A61 | 61 |
| A62 | 62 |
| A63 | 63 |
| A64 | 64 |

Preferred for better control of undesired vegetation (e.g., lower use rate such as from enhanced effects, broader spectrum of weeds controlled, or enhanced crop safety) or for preventing the development of resistant weeds are mixtures of a compound of this invention with a herbicide selected from the group consisting of chlorimuron-ethyl, nicosulfuron, mesotrione, thifensulfuron-methyl, flupyrsulfuron-methyl, tribenuron, pyroxasulfone, pinoxaden, tembotrione, pyroxsulam, metolachlor and S-metolachlor

The following Tests demonstrate the control efficacy of the compounds of this invention against specific weeds. The weed control afforded by the compounds is not limited, however, to these species. See Index Table A for compound descriptions. The following abbreviations are used in the Index Tables which follow: t is tertiary, s is secondary, n is normal, i is iso, c is cyclo, Me is methyl, Et is ethyl, Pr is propyl, i-Pr is isopropyl, Bu is butyl, c-Pr is cyclopropyl, c-Bu is cyclobutyl, c-Pen is cyclopentyl, t-Bu is tert-butyl, i-Bu is iso-butyl, Ph is phenyl, OMe is methoxy, OEt is ethoxy, SMe is methylthio, SEt is ethylthio, —CN is cyano, —NC2 is nitro, TMS is trimethylsilyl, allyl is CH₂CH═CH₂, propargyl is CH₂C═CH and naphthyl means naphthalenyl. Some other structures are defined in the table below.

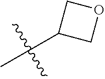 [SMILES: *C1COC1]

3-oxetanyl

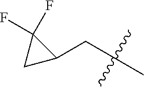 [SMILES: *CC1CC1(F)F]

2,2-difluorocyclopropylmethyl

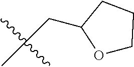 [SMILES: *CC1CCCO1]

tetrahydro-2-furanylmethyl

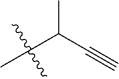 [SMILES: *C(C)C#C]

3-butyn-2-yl

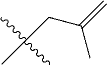 [SMILES: *CC(=C)C]

2-methyl-2-propen-1-yl

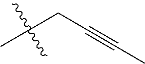 [SMILES: *CC#CC]

2-butyn-1-yl

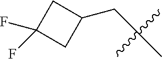 [SMILES: *CC1CC(F)(F)C1]

3,3-difluorocyclobutyl
methyl

(R) or (S) denotes the absolute chirality of the asymmetric carbon center. The abbreviation “(d)” indicates that the compound appeared to decompose on melting. The abbreviation “Cmpd. #” stands for “Compound Number”. The abbreviation “Ex.” stands for “Example” and is followed by a number indicating in which example the compound is prepared. Mass spectra are reported with an estimated precision within ±0.5 Da as the molecular weight of the highest isotopic abundance parent ion (M+1) formed by addition of H⁺ (molecular weight of 1) to the molecule observed by using atmospheric pressure chemical ionization (AP+).

INDEX TABLE A

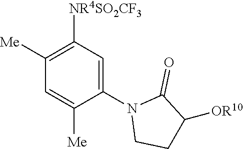

| Cmpd | | | M.S. | M.P. |
|---|---|---|---|---|
| # | R⁴ | R¹⁰ | | (° C.) |
| 1 | H | Propargyl | | 164-167 |
| 2 | H | Allyl | | 134-137 |
| 3 | H | c-Pr | | 171-174 |
| 4 | H | c-Bu | | 166-169 |
| 5 | CH₂OCO-t-Bu | c-Pen | 535.5 | |
| 6 | H | c-Pen | 421.4 | |
| 7 | CH₂OCO-t-Bu | c-Pr | 507.3 | |
| 8 | CH₂OCO-t-Bu | c-Bu | 521 | |
| 14 | H | H | | 226-229 |
| 15 | SO₂CF₃ | c-Pr | 525.3 | |
| 16 | CO₂Me | c-Pr | 451 | |
| 17 | COMe | c-Pr | 435 | |
| 18 | CO₂-i-Pr | c-Pr | 479 | |
| 19 | CO₂Et | c-Pr | 465 | |
| 20 | CH₂OCO-Pr | c-Pr | 493 | |
| 21 | CO₂-i-Bu | c-Pr | 493 | |
| 22 | H | CH₂-c-Pr | 407.3 | |
| 29* | H | c-Pr | | 169-172 |
| 30* | H | c-Pr | | 166-169 |
| 34 | H | 3-oxetanyl | 409.3 | |
| 41 | H | 2,2-difluorocyclopropylmethyl | 443.4 | |
| 42 | H | tetrahydro-2-furanylmethyl | 437.3 | |
| 43 | H | c-Bu-methyl | 421.4 | |
| 52 | H | 2,2-difluorocyclobutylmethyl | 457.5 | |
| 53 | CH₂OCO-t-Bu | c-Pr-methyl | 521 | |
| 54 | CH₂OCO-t-Bu | 2,2-difluorocyclopropylmethyl | 557 | |
| 55 | CH₂OCO-t-Bu | 3,3-difluorocyclobutyl methyl | 571 | |
| 56 | CH₂OCO-t-Bu | c-Bu-methyl | 535 | |
| 57 | H | c-Pen-methyl | | 147-150 |
| 58 | H | 3-butyn-2-yl | | 189-192 |
| 59 | H | 2-methyl-2-propen-1-yl | | 160-163 |
| 60 | H | 2-butyn-1-yl | | 129-132 |
| 61 | H | Benzyl | | 186-189 |
| 62 | SO₂CF₃ | 2-butyn-1-yl | | 101-104 |

*indicates that the compound is one of the following enantiomers.

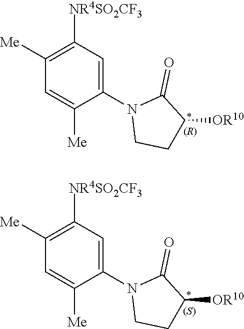

INDEX TABLE B

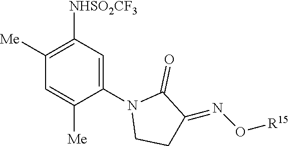

| Cmpd # | R¹⁵ | M.S. | M.P. (° C.) |
|---|---|---|---|
| 9 | Me | | 226-229 |
| 10 | H | | 213-216 |
| 11 | Allyl | | 184-187 |
| 12 | Et | | 201-204 |
| 13 | Propargyl | | 175-178 |

INDEX TABLE C

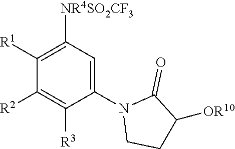

| Cmpd # | R¹ | R² | R³ | R⁴ | R¹⁰ | M.S. | M.P. (° C.) |
|---|---|---|---|---|---|---|---|
| 23 | Me | F | Me | CH₂OCO-t-Bu | c-Pr | | 110-113 |
| 24 | Me | F | Me | SO₂CF₃ | c-Pr | 543.3 | |
| 25 | Me | F | Me | H | c-Bu | | 149-152 |
| 26 | Me | F | Me | H | c-Pr | | 151-154 |
| 27 | Me | F | Me | SO₂CF₃ | c-Bu | | 60-63 |
| 28 | Me | F | Me | CH₂OCO-t-Bu | c-Bu | | 88-91 |
| 31 | Cl | H | Me | H | c-Pr | | 166-169 |
| 32 | H | H | Me | SO₂CF₃ | c-Pr | | 119-122 |
| 33 | Cl | H | Me | CH₂OCO-t-Bu | c-Pr | | 93-96 |
| 38 | Cl | H | Me | COMe | c-Pr | 455.24 | |
| 40 | Cl | H | Me | CO₂Me | c-Pr | | 71-74 |
| 44 | Me | Me | Me | SO₂CF₃ | c-Bu | 553.34 | |
| 45 | Me | Me | Me | H | c-Bu | | 197-200 |
| 46 | Me | Me | Me | SO₂CF₃ | c-Pr | | 127-130 |
| 47 | Me | Me | Me | H | c-Pr | | 193-196 |
| 48 | Me | Me | Me | CO₂-i-Bu | c-Bu | 521.49 | |
| 49 | Me | Me | Me | CH₂OCO-t-Bu | c-Pr | | 103-106 |
| 50 | Me | Me | Me | CO₂-i-Bu | c-Pr | 507.4 | |
| 51 | Me | Me | Me | CH₂OCO-t-Bu | c-Bu | 534.9 | |
| 63 | Me | F | Me | H | c-Pr-methyl | | 138-141 |
| 64 | Me | F | Me | H | c-Bu-methyl | | 146-149 |

INDEX TABLE D

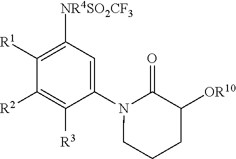

| Cmpd # | R¹ | R² | R³ | R⁴ | R¹⁰ | M.S. | M.P. (° C.) |
|---|---|---|---|---|---|---|---|
| 35 | Me | F | Me | H | c-Pr | | 211-214 |
| 36 | Me | F | Me | H | c-Bu | | 199-202 |
| 37 | Me | F | Me | CH₂OCO-t-Bu | c-Pr | | 86-89 |
| 39 | Me | F | Me | CH₂OCO-t-Bu | c-Bu | 553.36 | |

### BIOLOGICAL EXAMPLES OF THE INVENTION

### Test A

Seeds of plant species selected from barnyardgrass (Echinochloa crus-galli), blackgrass (Alopecurus myosuroides), corn (Zea mays), foxtail, giant (giant foxtail, Setaria faberi), goosegrass (Eleusine indica), kochia (Bassia scoparia), oat, wild (wild oat, Avena fatua), pigweed, palmer (palmer amaranth, Amaranthus palmeri), pigweed, redroot (redroot pigweed, Amaranthus retroflexus), ragweed (common ragweed, Ambrosia artemisiifolia), ryegrass, Italian (Italian ryegrass, Lolium multiflorum), soybean (Glycine max), and wheat (Triticum aestivum) were planted into a blend of loam soil and sand and treated preemergence with a directed soil spray using test chemicals formulated in a non-phytotoxic solvent mixture which included a surfactant.

At the same time, plants selected from these crop and weed species and also galium (catchweed bedstraw, Galium aparine) and horseweed (Erigeron canadensis) were planted in pots containing the same blend of loam soil and sand and treated with postemergence applications of test chemicals formulated in the same manner. Plants ranged in height from 2 to 10 cm and were in the one- to two-leaf stage for the postemergence treatment. Treated plants and untreated controls were maintained in a greenhouse for 10 days, after which time all treated plants were compared to untreated controls and visually evaluated for injury. Plant response ratings, summarized in Table A, are based on a 0 to 100 scale where 0 is no effect and 100 is complete control. A dash (-) response means no test result.

TABLE A

| | Compounds | | | | | | | |
|---|---|---|---|---|---|---|---|---|
| | 1 | 2 | 3 | 4 | 5 | 6 | 7 | 8 |
| 125 g ai/ha | | | | | | | | |
| Preemergence | | | | | | | | |
| Barnyardgrass | 100 | 100 | — | — | — | — | — | — |
| Blackgrass | — | — | 100 | 90 | 40 | 90 | 80 | 70 |
| Corn | — | — | 90 | 90 | 20 | 80 | 50 | 10 |
| Foxtail, Giant | 100 | 100 | 100 | 100 | 50 | 100 | 90 | 100 |
| Goosegrass | — | — | 100 | 100 | 90 | 100 | 100 | 100 |
| Kochia | 100 | 90 | 100 | 100 | 60 | 90 | 100 | 100 |
| Oat, Wild | — | — | 100 | 100 | 40 | 90 | 100 | 70 |
| Pigweed, Palmer | — | — | 100 | 100 | 50 | 100 | 100 | 90 |
| Pigweed, Redroot | 100 | 100 | — | — | — | — | — | — |
| Ragweed | 90 | 80 | 100 | 100 | 40 | 90 | 90 | 90 |
| Ryegrass, Italian | 100 | 100 | 100 | 100 | 90 | 90 | 100 | 90 |
| Soybean | — | — | 80 | 100 | 20 | 90 | 40 | 50 |
| Wheat | — | — | 100 | 100 | 20 | 100 | 80 | 50 |
| 31 g ai/ha | | | | | | | | |
| Preemergence | | | | | | | | |
| Barnyardgrass | 80 | 0 | — | — | — | — | — | — |
| Blackgrass | — | — | 80 | 20 | 0 | 50 | 30 | 30 |
| Corn | — | — | 90 | 40 | 0 | 30 | 0 | 0 |
| Foxtail, Giant | 90 | 100 | 100 | 100 | 0 | 100 | 90 | 90 |
| Goosegrass | — | — | 70 | 100 | 10 | 90 | 80 | 70 |
| Kochia | 70 | 50 | 100 | 70 | 0 | 30 | 60 | 0 |
| Oat, Wild | — | — | 70 | 80 | 0 | 70 | 20 | 0 |
| Pigweed, Palmer | — | — | 100 | 100 | 10 | 70 | 50 | 30 |
| Pigweed, Redroot | 90 | 90 | — | — | — | — | — | — |
| Ragweed | 30 | 20 | 90 | 90 | 0 | 30 | 30 | 20 |
| Ryegrass, Italian | 100 | 90 | 90 | 100 | 20 | 80 | 100 | 50 |
| Soybean | — | — | 30 | 70 | 0 | 50 | 20 | 40 |
| Wheat | — | — | 100 | 70 | 0 | 80 | 0 | 0 |
| 125 g ai/ha | | | | | | | | |
| Postemergence | | | | | | | | |
| Barnyardgrass | 90 | 90 | — | — | — | — | — | — |
| Blackgrass | 50 | 40 | 70 | — | 0 | 40 | 50 | 40 |
| Corn | 90 | 90 | 90 | 0 | 80 | 90 | 90 | 80 |
| Foxtail, Giant | 60 | 50 | 90 | 80 | 80 | 60 | 90 | 80 |
| Galium | 100 | 100 | 100 | 100 | 90 | 90 | 100 | 100 |
| Goosegrass | — | — | 90 | 90 | 90 | 90 | 90 | 90 |
| Horseweed | — | — | 90 | 100 | — | — | 90 | 90 |
| Kochia | 80 | 60 | 100 | 90 | 70 | 80 | 80 | 80 |
| Oat, Wild | — | — | 80 | 60 | 30 | 20 | 70 | 30 |
| Pigweed, Palmer | — | — | 60 | 90 | 70 | 60 | 60 | 90 |
| Pigweed, Redroot | 90 | 90 | — | — | — | — | — | — |
| Ragweed | 80 | 70 | 90 | 90 | 80 | 90 | 90 | 90 |
| Ryegrass, Italian | 70 | 70 | 90 | 80 | 40 | 50 | 90 | 100 |
| Soybean | — | — | 70 | 70 | 70 | 70 | 90 | 90 |
| Wheat | 70 | 70 | 80 | 50 | 50 | 60 | 60 | 70 |
| 31 g ai/ha | | | | | | | | |
| Postemergence | | | | | | | | |
| Barnyardgrass | 80 | 30 | — | — | — | — | — | — |
| Blackgrass | 10 | 20 | 40 | — | 0 | 0 | 20 | 30 |
| Corn | 50 | 0 | 90 | 90 | 0 | 0 | 90 | 30 |
| Foxtail, Giant | 40 | 30 | 70 | 40 | 0 | 10 | 60 | 30 |
| Galium | 80 | 80 | 70 | 100 | 60 | 80 | 90 | 100 |
| Goosegrass | — | — | 80 | 70 | 50 | 80 | 90 | 80 |
| Horseweed | — | — | 70 | 90 | — | — | 80 | 70 |
| Kochia | 30 | 20 | 50 | 60 | 20 | 20 | 70 | 60 |
| Oat, Wild | — | — | 40 | 30 | 20 | 0 | 30 | 10 |
| Pigweed, Palmer | — | — | 40 | 60 | 20 | 50 | 30 | 60 |
| Pigweed, Redroot | 80 | 90 | — | — | — | — | — | — |
| Ragweed | 60 | 50 | 80 | 70 | 60 | 60 | 90 | 70 |
| Ryegrass, Italian | 70 | 70 | 90 | 80 | 20 | 0 | 50 | 40 |
| Soybean | — | — | 60 | 50 | 60 | 70 | 90 | 60 |
| Wheat | 60 | 70 | 80 | 50 | 0 | 60 | 30 | 60 |

### Test B

Plant species in the flooded paddy test selected from barnyardgrass (Echinochloa crusgalli), ducksalad (Heteranthera limosa), rice (Oryza sativa), and sedge, umbrella (small-flower umbrella sedge, Cyperus difformis) were grown to the 2-leaf stage for testing. At time of treatment, test pots were flooded to 3 cm above the soil surface, treated by application of test compounds directly to the paddy water, and then maintained at that water depth for the duration of the test. Treated plants and controls were maintained in a greenhouse for 10 to 14 days, after which time all species were compared to controls and visually evaluated. Plant response ratings, summarized in Table B, are based on a scale of 0 to 100 where 0 is no effect and 100 is complete control. A dash (-) response means no test result.

TABLE B

| 250 g ai/ha | Compounds | | | | | | | |
|---|---|---|---|---|---|---|---|---|
| Flood | 1 | 2 | 3 | 4 | 5 | 6 | 7 | 8 |
| Barnyardgrass | 60 | 40 | 90 | 45 | 95 | 30 | 98 | 98 |
| Ducksalad | 80 | 75 | 98 | 100 | 95 | 85 | 95 | 95 |
| Rice | 30 | 10 | 50 | 0 | 0 | 10 | 30 | 35 |
| Sedge, Umbrella | 90 | 85 | 98 | 100 | 85 | 75 | 90 | 95 |

### Test C

Seeds of plant species selected from blackgrass (Alopecurus myosuroides), corn (Zea mays), foxtail, giant (giant foxtail, Setaria faberi), goosegrass (Eleusine indica), kochia (Bassia scoparia), oat, wild (wild oat, Avena fatua), pigweed, palmer (palmer amaranth, Amaranthus palmeri), ragweed (common ragweed, Ambrosia artemisiifolia), ryegrass, Italian (Italian ryegrass, Lolium multiflorum), soybean (Glycine max) and wheat (Triticum aestivum) were planted into a blend of loam soil and sand and treated preemergence with a directed soil spray using test chemicals formulated in a non-phytotoxic solvent mixture which included a surfactant.

At the same time, plants selected from these crop and weed species and also galium (catchweed bedstraw, Galium aparine) and horseweed (Erigeron canadensis) were planted in pots containing the same blend of loam soil and sand and treated with postemergence applications of test chemicals formulated in the same manner. Plants ranged in height from 2 to 10 cm and were in the one- to two-leaf stage for the postemergence treatment. Treated plants and untreated controls were maintained in a greenhouse for 10 or 12 days, after which time all treated plants were compared to untreated controls and visually evaluated for injury. Plant response ratings, summarized in Table A, are based on a 0 to 100 scale where 0 is no effect and 100 is complete control. A dash (-) response means no test result.

TABLE C

| | 125 g ai/ha Preemergence | | | | | | | | | | | | | |
|---|---|---|---|---|---|---|---|---|---|---|---|---|---|---|
| | Compounds | | | | | | | | | | | | | |
| | 9 | 10 | 11 | 12 | 13 | 14 | 16 | 17 | 18 | 19 | 20 | 21 | 22 | 23 |
| Blackgrass | — | — | 100 | 100 | 100 | 90 | 0 | 90 | 0 | 40 | 100 | 80 | 90 | 10 |
| Corn | 100 | 90 | 100 | 100 | 100 | 100 | 0 | 90 | 0 | 0 | 90 | 90 | 90 | 20 |
| Foxtail, Giant | 100 | 100 | 100 | 100 | 100 | 100 | 0 | 100 | 0 | 10 | 100 | 100 | 100 | 90 |
| Goosegrass | 100 | 100 | 100 | 100 | 100 | 100 | 0 | 100 | 0 | 70 | 100 | 100 | 100 | 100 |
| Kochia | 100 | 100 | 100 | 100 | 100 | 100 | 0 | 100 | 60 | 70 | 100 | 90 | 100 | 90 |
| Oat, Wild | 100 | 100 | 100 | 100 | 100 | 60 | 0 | 100 | 0 | 40 | 100 | 90 | 100 | 90 |
| Pigweed, Palmer | 100 | 100 | 100 | 100 | 100 | 100 | 0 | 100 | 20 | 60 | 100 | 100 | 100 | 100 |
| Ragweed | 90 | 30 | 90 | 100 | 100 | 60 | 0 | 90 | 0 | 10 | 90 | 80 | 100 | 90 |
| Ryegrass, Italian | 100 | 100 | 90 | 100 | 100 | 100 | 0 | 100 | 0 | 0 | 100 | 100 | 100 | 100 |
| Soybean | 100 | 70 | 90 | 100 | 100 | 100 | 20 | 100 | 30 | 90 | 90 | 60 | 90 | 40 |
| Wheat | 100 | 100 | 100 | 100 | 100 | 100 | 0 | 100 | 0 | 10 | 100 | 90 | 100 | 30 |

| | 125 g ai/ha Preemergence | | | | | | | | | | | | | |
|---|---|---|---|---|---|---|---|---|---|---|---|---|---|---|
| | Compounds | | | | | | | | | | | | | |
| | 25 | 26 | 28 | 29 | 30 | 31 | 33 | 34 | 35 | 36 | 37 | 38 | 39 | 40 |
| Blackgrass | 90 | 90 | 60 | 90 | 100 | 80 | 80 | 50 | 100 | 100 | 60 | 90 | 20 | 90 |
| Corn | 90 | 90 | 20 | 90 | 90 | 70 | 60 | 80 | 50 | 90 | 10 | 80 | 10 | 90 |
| Foxtail, Giant | 100 | 100 | 100 | 100 | 100 | 100 | 100 | 90 | 100 | 100 | 90 | 100 | 10 | 100 |
| Goosegrass | 100 | 100 | 100 | 100 | 100 | 100 | 100 | 100 | 100 | 100 | 100 | 100 | 60 | 100 |
| Kochia | 100 | 100 | 90 | 100 | 100 | 100 | 100 | 100 | 80 | 90 | 90 | 100 | 60 | 100 |
| Oat, Wild | 90 | 100 | 20 | 90 | 100 | 90 | 50 | 80 | 90 | 90 | 20 | 100 | 0 | 90 |
| Pigweed, Palmer | 100 | 100 | 80 | 100 | 100 | 100 | 80 | 100 | 100 | 100 | 90 | 100 | 40 | 100 |
| Ragweed | 100 | 100 | 100 | 90 | 100 | 100 | 80 | 100 | 90 | 90 | 90 | 90 | 80 | 90 |
| Ryegrass, Italian | 100 | 100 | 90 | 100 | 100 | 100 | 100 | 80 | 100 | 100 | 70 | 100 | 60 | 100 |
| Soybean | 90 | 90 | 80 | 100 | 90 | 90 | 60 | 90 | 90 | 90 | 30 | 90 | 60 | 90 |
| Wheat | 100 | 100 | 30 | 100 | 100 | 100 | 40 | 80 | 100 | 100 | 20 | 90 | 0 | 100 |

| | 125 g ai/ha Preemergence | | | | | | | | | | | | | |
|---|---|---|---|---|---|---|---|---|---|---|---|---|---|---|
| | Compounds | | | | | | | | | | | | | |
| | 41 | 42 | 43 | 45 | 47 | 48 | 49 | 50 | 51 | 52 | 53 | 54 | 55 | 56 |
| Blackgrass | 100 | 70 | 50 | 90 | — | — | — | — | — | — | — | — | — | — |
| Corn | 100 | 70 | 100 | 100 | 90 | 60 | 50 | 90 | 40 | 90 | 50 | 30 | 10 | 10 |
| Foxtail, Giant | 100 | 100 | 100 | 100 | 100 | 100 | 90 | 100 | 90 | 100 | 90 | 80 | 10 | 80 |
| Goosegrass | 100 | 100 | 100 | 100 | 100 | 100 | 90 | 100 | 90 | 100 | 100 | 100 | 100 | 90 |
| Kochia | 100 | 90 | 80 | 100 | 100 | 90 | 70 | 100 | 80 | 100 | 50 | 30 | 10 | 70 |
| Oat, Wild | 100 | 30 | 50 | 90 | 100 | 80 | 90 | 80 | 50 | 90 | 90 | 80 | 20 | 0 |
| Pigweed, Palmer | 100 | 100 | 100 | 100 | 100 | 100 | 100 | 100 | 60 | 100 | 90 | 50 | 30 | 90 |
| Ragweed | 90 | 60 | 60 | 100 | 100 | 90 | 100 | 90 | 70 | 30 | 70 | 60 | 40 | 30 |
| Ryegrass, Italian | 100 | 90 | 100 | 90 | 90 | 100 | 100 | 100 | 100 | 80 | 100 | 90 | 60 | 80 |
| Soybean | 100 | 90 | 90 | 100 | 90 | 70 | 40 | 60 | 30 | 50 | 40 | 30 | 0 | 30 |
| Wheat | 100 | 90 | 100 | 100 | 100 | 80 | 90 | 100 | 60 | 100 | 90 | 80 | 40 | 0 |

| | 125 g ai/ha Preemergence | | | | | | |
|---|---|---|---|---|---|---|---|
| | Compounds | | | | | | |
| | 57 | 58 | 59 | 60 | 61 | 63 | 64 |
| Blackgrass | 80 | 100 | 90 | 90 | 90 | 90 | 70 |
| Corn | 50 | 100 | 100 | 100 | 100 | 100 | 60 |
| Foxtail, Giant | 100 | 100 | 100 | 100 | 100 | 100 | 100 |
| Goosegrass | 100 | 100 | 100 | 100 | 100 | 100 | 100 |
| Kochia | 80 | 90 | 100 | 100 | 100 | 100 | 90 |
| Oat, Wild | 80 | 100 | 100 | 100 | 90 | 100 | 90 |
| Pigweed, Palmer | 100 | 100 | 100 | 100 | 100 | 100 | 100 |
| Ragweed | 80 | 90 | 70 | 100 | 90 | 100 | 100 |
| Ryegrass, Italian | 90 | 100 | 100 | 100 | 90 | 100 | 90 |
| Soybean | 90 | 100 | 90 | 90 | 90 | 100 | 90 |
| Wheat | 80 | 100 | 100 | 100 | 100 | 100 | 70 |

| | 31 g ai/ha Preemergence | | | | | | | | | | | | | |
|---|---|---|---|---|---|---|---|---|---|---|---|---|---|---|
| | Compounds | | | | | | | | | | | | | |
| | 9 | 10 | 11 | 12 | 13 | 14 | 16 | 17 | 18 | 19 | 20 | 21 | 22 | 23 |
| Blackgrass | — | — | 60 | 80 | 90 | 60 | 0 | 80 | 0 | 0 | 80 | 10 | 50 | 0 |
| Corn | 50 | 30 | 50 | 70 | 40 | 10 | 0 | 70 | 0 | 0 | 60 | 20 | 90 | 10 |
| Foxtail, Giant | 100 | 90 | 100 | 100 | 100 | 80 | 0 | 90 | 0 | 0 | 90 | 100 | 100 | 20 |
| Goosegrass | 100 | 80 | 100 | 90 | 90 | 90 | 0 | 100 | 0 | 0 | 100 | 90 | 100 | 70 |
| Kochia | 100 | 90 | 80 | 100 | 100 | 50 | 0 | 80 | 0 | 0 | 80 | 60 | 90 | 20 |
| Oat, Wild | 90 | 90 | 30 | 90 | 100 | 10 | 0 | 90 | 0 | 0 | 60 | 30 | 90 | 40 |
| Pigweed, Palmer | 100 | 50 | 70 | 100 | 100 | 90 | 0 | 80 | 0 | 20 | 90 | 100 | 100 | 30 |
| Ragweed | 50 | 0 | 80 | 100 | 50 | 20 | 0 | 80 | 0 | 0 | 60 | 40 | 50 | 30 |
| Ryegrass, Italian | 60 | 50 | 40 | 90 | 40 | 40 | 0 | 100 | 0 | 0 | 100 | 70 | 100 | 30 |
| Soybean | 80 | 60 | 50 | 90 | 80 | 100 | 20 | 80 | 0 | 30 | 70 | 0 | 80 | 30 |
| Wheat | 90 | 90 | 90 | 90 | 90 | 50 | 0 | 30 | 0 | 0 | 70 | 10 | 90 | 10 |

| | 31 g ai/ha Preemergence | | | | | | | | | | | | | |
|---|---|---|---|---|---|---|---|---|---|---|---|---|---|---|
| | Compound | | | | | | | | | | | | | |
| | 25 | 26 | 28 | 29 | 30 | 31 | 33 | 34 | 35 | 36 | 37 | 38 | 39 | 40 |
| Blackgrass | 80 | 70 | 50 | 90 | 80 | 60 | 20 | 30 | 70 | 80 | 10 | 80 | 0 | 50 |
| Corn | 30 | 60 | 0 | 80 | 90 | 30 | 30 | 10 | 30 | 30 | 0 | 20 | 0 | 50 |
| Foxtail, Giant | 100 | 90 | 70 | 90 | 100 | 90 | 70 | 90 | 90 | 50 | 20 | 80 | 0 | 100 |
| Goosegrass | 90 | 90 | 70 | 100 | 90 | 90 | 90 | 30 | 100 | 90 | 50 | 100 | 10 | 100 |
| Kochia | 70 | 100 | 20 | 90 | 90 | 100 | 100 | 90 | 20 | 20 | 0 | 100 | 20 | 90 |
| Oat, Wild | 40 | 60 | 0 | 90 | 80 | 60 | 10 | 30 | 20 | 30 | 20 | 30 | 0 | 10 |
| Pigweed, Palmer | 100 | 100 | 30 | 100 | 100 | 90 | 70 | 100 | 100 | 90 | 50 | 90 | 0 | 80 |
| Ragweed | 90 | 90 | 40 | 80 | 100 | 90 | 30 | 50 | 50 | 70 | 0 | 70 | 0 | 70 |
| Ryegrass, Italian | 90 | 90 | 30 | 100 | 100 | 60 | 20 | 40 | 100 | 100 | 30 | 80 | 0 | 90 |
| Soybean | 80 | 70 | 20 | 90 | 80 | 70 | 0 | 40 | 50 | 90 | 0 | 80 | 0 | 70 |
| Wheat | 60 | 60 | 20 | 90 | 90 | 30 | 10 | 30 | 20 | 40 | 0 | 40 | 0 | 60 |

| | 31 g ai/ha Preemergence | | | | | | | | | | | | | |
|---|---|---|---|---|---|---|---|---|---|---|---|---|---|---|
| | Compounds | | | | | | | | | | | | | |
| | 41 | 42 | 43 | 45 | 47 | 48 | 49 | 50 | 51 | 52 | 53 | 54 | 55 | 56 |
| Blackgrass | 80 | 20 | 40 | 60 | — | — | — | — | — | — | — | — | — | — |
| Corn | 90 | 0 | 40 | 90 | 30 | 20 | 0 | 10 | 0 | 10 | 0 | 0 | 0 | 0 |
| Foxtail, Giant | 100 | 20 | 30 | 100 | 100 | 30 | 20 | 50 | 10 | 90 | 10 | 0 | 0 | 0 |
| Goosegrass | 90 | 50 | 60 | 100 | 100 | 80 | 60 | 90 | 0 | 80 | 60 | 20 | 10 | 10 |
| Kochia | 100 | 20 | 60 | 90 | 70 | 10 | 30 | 20 | 30 | 70 | 0 | 0 | 0 | 0 |
| Oat, Wild | 60 | 0 | 0 | 80 | 80 | 50 | 60 | 70 | 20 | 20 | 0 | 0 | 0 | 0 |
| Pigweed, Palmer | 100 | 80 | 70 | 100 | 100 | 100 | 30 | 90 | 20 | 70 | 30 | 0 | 0 | 10 |
| Ragweed | 70 | 10 | 10 | 80 | 70 | 20 | 100 | 30 | 0 | 0 | 60 | 0 | — | 0 |
| Ryegrass, Italian | 100 | 20 | 80 | 50 | 50 | 60 | 40 | 50 | 40 | 0 | 60 | 0 | 20 | 30 |
| Soybean | 60 | 0 | 10 | 50 | 50 | 30 | 0 | 30 | 0 | 30 | 20 | 0 | 0 | 0 |
| Wheat | 90 | 30 | 20 | 60 | 90 | 30 | 30 | 30 | 10 | 90 | 60 | 0 | 10 | 0 |

| | 31 g ai/ha Preemergence | | | | | | |
|---|---|---|---|---|---|---|---|
| | Compounds | | | | | | |
| | 57 | 58 | 59 | 60 | 61 | 63 | 64 |
| Blackgrass | 40 | 80 | 70 | 80 | 60 | 70 | 20 |
| Corn | 0 | 20 | 40 | 30 | 20 | 30 | 10 |
| Foxtail, Giant | 80 | 100 | 100 | 100 | 100 | 100 | 100 |
| Goosegrass | 90 | 100 | 90 | 90 | 100 | 100 | 70 |
| Kochia | 60 | 80 | 80 | 90 | 90 | 90 | 70 |
| Oat, Wild | 20 | 90 | 80 | 90 | 50 | 70 | 20 |
| Pigweed, Palmer | 100 | 100 | 100 | 100 | 100 | 100 | 100 |
| Ragweed | 0 | 80 | 50 | 90 | 0 | 90 | 90 |
| Ryegrass, Italian | 70 | 90 | 30 | 70 | 60 | 50 | 50 |
| Soybean | 70 | 100 | 60 | 50 | 40 | 60 | 40 |
| Wheat | 20 | 90 | 40 | 40 | 30 | 80 | 20 |

| | 125 g ai/ha Postemergence | | | | | | | | | | | | | |
|---|---|---|---|---|---|---|---|---|---|---|---|---|---|---|
| | Compounds | | | | | | | | | | | | | |
| | 9 | 10 | 11 | 12 | 13 | 14 | 16 | 17 | 18 | 19 | 20 | 21 | 22 | 23 |
| Blackgrass | 90 | 90 | 90 | 100 | 90 | 70 | 0 | 80 | 0 | 40 | 90 | 80 | 90 | 50 |
| Corn | 90 | 90 | 100 | 100 | 100 | 90 | 0 | 90 | 0 | 20 | 90 | 90 | 90 | 90 |
| Foxtail, Giant | 90 | 90 | 90 | 90 | 90 | 90 | 0 | 90 | 0 | 20 | 90 | 90 | 60 | 80 |
| Galium | 100 | 100 | 100 | 100 | 100 | 100 | 50 | 100 | 70 | 90 | 100 | 100 | 100 | 100 |
| Goosegrass | 90 | 90 | 100 | 100 | 100 | 90 | 0 | 90 | 0 | 90 | 90 | 90 | 90 | 90 |
| Horseweed | 100 | 90 | 70 | 100 | 90 | 90 | 30 | 100 | 0 | 30 | 90 | 90 | 90 | 90 |
| Kochia | 90 | 90 | 90 | 90 | 90 | 70 | 10 | 90 | 70 | 70 | 80 | 80 | 70 | 80 |
| Oat, Wild | 80 | 80 | — | 90 | 90 | 60 | 0 | 70 | 0 | 30 | 80 | 70 | 40 | 50 |
| Pigweed, Palmer | 90 | 90 | 90 | 100 | 90 | 90 | 0 | 90 | 20 | 10 | 80 | 70 | 80 | 70 |
| Ragweed | 80 | 50 | 80 | 90 | 90 | 80 | 90 | 90 | 50 | 90 | 90 | 90 | 80 | 90 |
| Ryegrass, Italian | 80 | 80 | 30 | 80 | 60 | 100 | 0 | 90 | 0 | 50 | 100 | 100 | 80 | 100 |
| Soybean | 80 | 80 | 100 | 100 | 100 | 80 | 60 | 80 | 80 | 80 | 70 | 60 | 70 | 60 |
| Wheat | 80 | 80 | 90 | 90 | 90 | 60 | 0 | 70 | 0 | 10 | 70 | 50 | 60 | 60 |

| | 125 g ai/ha Postemergence | | | | | | | | | | | | | |
|---|---|---|---|---|---|---|---|---|---|---|---|---|---|---|
| | Compounds | | | | | | | | | | | | | |
| | 25 | 26 | 28 | 29 | 30 | 31 | 33 | 34 | 35 | 36 | 37 | 38 | 39 | 40 |
| Blackgrass | 90 | 90 | 70 | 70 | 90 | 100 | 60 | 60 | 60 | 30 | 60 | 70 | 10 | 60 |
| Corn | 60 | 90 | 30 | 90 | 90 | 90 | 90 | 90 | 90 | 20 | 90 | 90 | 10 | 100 |
| Foxtail, Giant | 30 | 90 | 70 | 90 | 80 | 90 | 90 | 90 | 90 | 30 | 80 | 90 | 10 | 90 |
| Galium | 100 | 100 | 100 | 100 | 100 | 100 | 100 | 100 | 100 | 80 | 90 | 100 | 90 | 100 |
| Goosegrass | 90 | 100 | 70 | 90 | 90 | 100 | 90 | 80 | 90 | 90 | 90 | 90 | 30 | 90 |
| Horseweed | — | — | — | — | — | 100 | 100 | 100 | 100 | 70 | 80 | 100 | 90 | 80 |
| Kochia | 90 | 90 | 80 | 90 | 90 | 90 | 90 | 90 | 90 | 80 | 70 | 90 | 60 | 90 |
| Oat, Wild | 20 | 50 | 30 | 80 | 80 | 90 | 70 | 70 | 70 | 30 | 70 | 60 | 20 | 80 |
| Pigweed, Palmer | 90 | 90 | 90 | 90 | 90 | 90 | 80 | 100 | 70 | 70 | 30 | 80 | 20 | 90 |
| Ragweed | 80 | 80 | 80 | 80 | 90 | 90 | 90 | 90 | 90 | 90 | 90 | 90 | 80 | 80 |
| Ryegrass, Italian | 70 | 100 | 40 | 100 | 100 | 90 | 90 | 90 | 60 | 40 | 60 | 70 | 30 | 80 |
| Soybean | 100 | 100 | 60 | 100 | 60 | 70 | 100 | 70 | 60 | 70 | 70 | 80 | 90 | 80 |
| Wheat | 60 | 70 | 60 | 70 | 70 | 80 | 70 | 80 | 60 | 60 | 60 | 60 | 60 | 70 |

| | 125 g ai/ha Postemergence | | | | | | | | | | | | | |
|---|---|---|---|---|---|---|---|---|---|---|---|---|---|---|
| | Compounds | | | | | | | | | | | | | |
| | 41 | 42 | 43 | 45 | 47 | 48 | 49 | 50 | 51 | 52 | 53 | 54 | 55 | 56 |
| Blackgrass | 60 | 50 | 50 | 40 | 70 | 30 | 60 | 40 | 20 | 40 | 60 | 90 | 40 | 20 |
| Corn | 90 | 90 | 90 | 90 | 90 | 50 | 100 | 90 | 60 | 60 | 60 | 80 | 70 | 30 |
| Foxtail, Giant | 90 | 90 | 90 | 80 | 90 | 60 | 90 | 80 | 40 | 50 | 90 | 70 | 70 | 40 |
| Galium | 100 | 90 | 90 | 100 | 90 | 100 | 90 | 100 | 90 | 100 | — | — | — | — |
| Goosegrass | 90 | 90 | 90 | 90 | 90 | 40 | 80 | 80 | 20 | 70 | 90 | 90 | 90 | 60 |
| Horseweed | 100 | 80 | 60 | 90 | 100 | 90 | 100 | — | 90 | 90 | 90 | 90 | 80 | 80 |
| Kochia | 80 | 60 | 60 | 100 | 80 | 60 | 60 | 70 | 70 | 70 | 50 | 50 | 20 | 30 |
| Oat, Wild | 30 | 40 | 20 | 30 | 60 | 20 | 70 | 50 | 30 | 60 | 50 | 80 | 30 | 10 |
| Pigweed, Palmer | 90 | 70 | 90 | 90 | 70 | 70 | 50 | 60 | 60 | 40 | 70 | 50 | 40 | 40 |
| Ragweed | 70 | 90 | 90 | 80 | 80 | 80 | 90 | 80 | 80 | 70 | 90 | 80 | 80 | 60 |
| Ryegrass, Italian | 100 | 70 | 70 | 50 | 90 | 60 | 90 | 90 | 50 | 100 | 70 | 70 | 60 | 60 |
| Soybean | 90 | 80 | 90 | 100 | 90 | 70 | 80 | 80 | 60 | 70 | 60 | 60 | 70 | 50 |
| Wheat | 60 | 80 | 70 | 60 | 80 | 70 | 70 | 80 | 70 | 70 | 80 | 70 | 70 | 70 |

| | 125 g ai/ha Postemergence | | | | | | |
|---|---|---|---|---|---|---|---|
| | Compounds | | | | | | |
| | 57 | 58 | 59 | 60 | 61 | 63 | 64 |
| Blackgrass | 80 | 70 | 80 | 90 | 70 | 30 | 20 |
| Corn | 40 | 90 | 90 | 100 | 60 | 50 | 10 |
| Foxtail, Giant | 70 | 90 | 90 | 90 | 50 | 30 | 20 |
| Galium | 90 | 90 | 100 | 100 | 90 | 100 | 100 |
| Goosegrass | 90 | 100 | 90 | 90 | 90 | 90 | 70 |
| Horseweed | 80 | 90 | 80 | 100 | 60 | 90 | 60 |
| Kochia | 70 | 90 | 80 | 90 | 90 | 90 | 100 |
| Oat, Wild | 10 | 90 | 90 | 90 | 80 | 40 | 10 |
| Pigweed, Palmer | 70 | 80 | 80 | 90 | 80 | 90 | 70 |
| Ragweed | 60 | 90 | 70 | 90 | 80 | 80 | 70 |
| Ryegrass, Italian | 60 | 70 | 70 | 70 | 60 | 10 | 20 |
| Soybean | 100 | 90 | 80 | 100 | 90 | 100 | 90 |
| Wheat | 70 | 70 | 80 | 90 | 80 | 40 | 20 |

| | 31 g ai/ha Postemergence | | | | | | | | | | | | | |
|---|---|---|---|---|---|---|---|---|---|---|---|---|---|---|
| | Compounds | | | | | | | | | | | | | |
| | 9 | 10 | 11 | 12 | 13 | 14 | 16 | 17 | 18 | 19 | 20 | 21 | 22 | 23 |
| Blackgrass | 80 | 60 | 40 | 80 | 80 | 10 | 0 | 70 | 0 | 0 | 90 | 40 | 20 | 10 |
| Corn | 90 | 90 | 90 | 100 | 100 | 70 | 0 | 90 | 0 | 0 | 90 | 90 | 50 | 30 |
| Foxtail, Giant | 40 | 40 | 60 | 90 | 60 | 20 | 0 | 90 | 0 | 0 | 80 | 40 | 20 | 0 |
| Galium | 90 | 100 | 80 | 100 | 100 | 100 | 50 | 90 | 10 | 30 | 90 | 100 | 100 | 90 |
| Goosegrass | 90 | 90 | 90 | 100 | 70 | 60 | 0 | 90 | 0 | 0 | 90 | 90 | 70 | 30 |
| Horseweed | 100 | 80 | 20 | 90 | 70 | 90 | 0 | 70 | 0 | 10 | 60 | 60 | 90 | 80 |
| Kochia | 80 | 60 | 40 | 90 | 90 | 50 | 20 | 80 | 20 | 30 | 70 | 70 | 60 | 30 |
| Oat, Wild | 80 | 80 | — | 90 | 90 | 30 | 0 | 30 | 0 | 0 | 30 | 40 | 20 | 20 |
| Pigweed, Palmer | 70 | 60 | 40 | 80 | 80 | 40 | 0 | 40 | 0 | 0 | 50 | 30 | 50 | 50 |
| Ragweed | 70 | 50 | 60 | 90 | 80 | 60 | 80 | 70 | 0 | 60 | 80 | 80 | 60 | 90 |
| Ryegrass, Italian | 50 | 80 | 0 | 70 | 60 | 60 | 0 | 60 | 0 | 0 | 60 | 50 | 70 | 50 |
| Soybean | 80 | 80 | 100 | 100 | 100 | 80 | 40 | 60 | 50 | 80 | 60 | 60 | 60 | 60 |
| Wheat | 80 | 80 | 80 | 80 | 90 | 60 | 0 | 70 | 0 | 0 | 70 | 10 | 50 | 10 |

| | 31 g ai/ha Postemergence | | | | | | | | | | | | | |
|---|---|---|---|---|---|---|---|---|---|---|---|---|---|---|
| | Compounds | | | | | | | | | | | | | |
| | 25 | 26 | 28 | 29 | 30 | 31 | 33 | 34 | 35 | 36 | 37 | 38 | 39 | 40 |
| Blackgrass | 0 | 40 | 10 | 60 | 50 | 50 | 0 | 30 | 50 | 20 | 0 | 40 | 0 | 10 |
| Corn | 30 | 50 | 10 | 80 | 90 | 70 | 80 | 60 | 50 | 0 | 10 | 80 | 0 | 80 |
| Foxtail, Giant | 30 | 40 | 30 | 90 | 50 | 30 | 50 | 40 | 40 | 10 | 10 | 50 | 0 | 50 |
| Galium | 100 | 100 | 90 | 80 | 80 | 100 | 100 | 100 | 70 | 60 | 60 | 90 | 80 | 80 |
| Goosegrass | 50 | 80 | 30 | 90 | 80 | 60 | 60 | 30 | 70 | 30 | 0 | 30 | 0 | 50 |
| Horseweed | — | — | — | — | — | 100 | 70 | 90 | 90 | 40 | 80 | 90 | 70 | 80 |
| Kochia | 60 | 70 | 50 | 60 | 80 | 80 | 80 | 80 | 50 | 40 | 50 | 80 | 30 | 70 |
| Oat, Wild | 0 | 20 | 20 | 20 | 50 | 60 | 30 | 30 | 40 | 30 | 20 | 50 | 0 | 50 |
| Pigweed, Palmer | 70 | 70 | 50 | 60 | 60 | 70 | 20 | 70 | 40 | 30 | 0 | 50 | 0 | 30 |
| Ragweed | 60 | 60 | 80 | 60 | 90 | 90 | 90 | 90 | 60 | 40 | 60 | 80 | 30 | 60 |
| Ryegrass, Italian | 20 | 60 | 40 | 70 | 90 | 40 | 20 | 40 | 50 | 0 | 0 | 30 | 0 | 60 |
| Soybean | 100 | 70 | 50 | 60 | 50 | 60 | 80 | 60 | 60 | 70 | 60 | 70 | 50 | 70 |
| Wheat | 50 | 60 | 20 | 70 | 70 | 60 | 50 | 70 | 60 | 60 | 10 | 60 | 0 | 60 |

| | 31 g ai/ha Postemergence | | | | | | | | | | | | | |
|---|---|---|---|---|---|---|---|---|---|---|---|---|---|---|
| | Compounds | | | | | | | | | | | | | |
| | 41 | 42 | 43 | 45 | 47 | 48 | 49 | 50 | 51 | 52 | 53 | 54 | 55 | 56 |
| Blackgrass | 40 | 0 | 0 | 10 | 40 | 10 | 30 | 10 | 10 | 20 | 20 | 30 | 0 | 0 |
| Corn | 40 | 20 | 40 | 20 | 70 | 0 | 50 | 30 | 0 | 0 | 20 | 0 | 0 | 0 |
| Foxtail, Giant | 20 | 20 | 10 | 30 | 70 | 20 | 60 | 50 | 0 | 0 | 30 | 30 | 30 | 10 |
| Galium | 100 | 50 | 70 | 100 | 80 | 90 | 80 | 60 | 60 | 70 | — | — | — | — |
| Goosegrass | 90 | 40 | 20 | 70 | 50 | 0 | 20 | 20 | 0 | 0 | 60 | 60 | 40 | 0 |
| Horseweed | 70 | 20 | 30 | 30 | — | 70 | — | — | 60 | 80 | 80 | 70 | 80 | 20 |
| Kochia | 70 | 10 | 20 | 70 | 40 | 20 | 40 | 20 | 30 | 20 | 20 | 30 | 0 | 0 |
| Oat, Wild | 30 | 0 | 10 | 10 | 10 | 10 | 30 | 20 | 0 | 10 | 10 | 10 | 20 | 10 |
| Pigweed, Palmer | 50 | 50 | 40 | 70 | 30 | 30 | 10 | 20 | 10 | 0 | 40 | 0 | 0 | 0 |
| Ragweed | 50 | 40 | 50 | 50 | 60 | 30 | 70 | 70 | 60 | 40 | 70 | 50 | 50 | 20 |
| Ryegrass, Italian | 30 | 20 | 30 | 0 | 50 | 40 | 50 | 50 | 20 | 40 | 0 | 60 | 0 | 0 |
| Soybean | 90 | 70 | 80 | 80 | 70 | 30 | 60 | 60 | 30 | 60 | 50 | 50 | 50 | 40 |
| Wheat | 60 | 0 | 30 | 20 | 80 | 30 | 70 | 60 | 20 | 20 | 40 | 30 | 20 | 0 |

| | 31 g ai/ha Postemergence | | | | | | |
|---|---|---|---|---|---|---|---|
| | Compounds | | | | | | |
| | 57 | 58 | 59 | 60 | 61 | 63 | 64 |
| Blackgrass | 40 | 50 | 10 | 50 | 30 | 0 | 0 |
| Corn | 0 | 60 | 20 | 90 | 10 | 0 | 0 |
| Foxtail, Giant | 60 | 60 | 10 | 80 | 20 | 0 | 0 |
| Galium | 50 | 90 | 100 | 100 | 70 | 100 | 100 |
| Goosegrass | 70 | 100 | 70 | 80 | 40 | 0 | 30 |
| Horseweed | 0 | 90 | 20 | 80 | 60 | 90 | 10 |
| Kochia | 50 | 70 | 70 | 100 | 60 | 80 | 50 |
| Oat, Wild | 10 | 60 | 30 | 30 | 20 | 0 | 0 |
| Pigweed, Palmer | 40 | 50 | 40 | 60 | 50 | 70 | 60 |
| Ragweed | 40 | 70 | 10 | 80 | 50 | 40 | 30 |
| Ryegrass, Italian | 20 | 60 | 10 | 50 | 0 | 0 | 0 |
| Soybean | 80 | 60 | 60 | 80 | 60 | 50 | 60 |
| Wheat | 70 | 70 | 50 | 80 | 70 | 10 | 0 |

### Test D

Plant species in the flooded paddy test selected from barnyardgrass (Echinochloa crusgalli), ducksalad (Heteranthera limosa), rice (Oryza sativa), and sedge, umbrella (small-flower umbrella sedge, Cyperus difformis) were grown to the 2-leaf stage for testing. At time of treatment, test pots were flooded to 3 cm above the soil surface, treated by application of test compounds directly to the paddy water, and then maintained at that water depth for the duration of the test. Treated plants and controls were maintained in a greenhouse for 13 days, after which time all species were compared to controls and visually evaluated. Plant response ratings, summarized in Table B, are based on a scale of 0 to 100 where 0 is no effect and 100 is complete control. A dash (-) response means no test result.

TABLE D

| | 250 g ai/ha Flood | | | | | | | | | | | | | |
|---|---|---|---|---|---|---|---|---|---|---|---|---|---|---|
| | Compounds | | | | | | | | | | | | | |
| | 9 | 10 | 11 | 12 | 13 | 14 | 16 | 17 | 18 | 19 | 20 | 21 | 22 | 23 |
| Barnyardgrass | 10 | 0 | 25 | 0 | 10 | 0 | 0 | 85 | 0 | 0 | 90 | 95 | 90 | 95 |
| Ducksalad | 95 | 90 | 100 | 100 | 100 | 65 | 0 | 95 | 0 | 0 | 90 | 98 | 90 | 80 |
| Rice | 30 | 0 | 45 | 40 | 35 | 25 | 0 | 15 | 0 | 0 | 15 | 85 | 55 | 40 |
| Sedge, Umbrella | 85 | 80 | 100 | 100 | 100 | 35 | 0 | 95 | 0 | 0 | 98 | 98 | 95 | 95 |

| | 250 g ai/ha Flood | | | | | | | | | | | | | |
|---|---|---|---|---|---|---|---|---|---|---|---|---|---|---|
| | Compounds | | | | | | | | | | | | | |
| | 25 | 26 | 28 | 29 | 30 | 31 | 33 | 34 | 35 | 36 | 37 | 38 | 39 | 40 |
| Barnyardgrass | 0 | 30 | 60 | 95 | 95 | 30 | 90 | 35 | 0 | 0 | 90 | 0 | 35 | 0 |
| Ducksalad | 75 | 75 | 70 | 45 | 90 | 75 | 80 | 65 | 55 | 65 | 70 | 65 | 55 | 70 |
| Rice | 15 | 65 | 30 | 85 | 75 | 30 | 45 | 25 | 0 | 0 | 0 | 0 | 0 | 0 |
| Sedge, Umbrella | 90 | 95 | 95 | 95 | 98 | 80 | 90 | 80 | 95 | 95 | 95 | 75 | 95 | 90 |

| | 250 g ai/ha Flood | | | | | | | | | | | | | |
|---|---|---|---|---|---|---|---|---|---|---|---|---|---|---|
| | Compounds | | | | | | | | | | | | | |
| | 41 | 42 | 43 | 45 | 47 | 48 | 49 | 50 | 51 | 52 | 53 | 54 | 55 | 56 |
| Barnyardgrass | 15 | 0 | 25 | 95 | 75 | 80 | 100 | 90 | 100 | 20 | 40 | 45 | 50 | 35 |
| Ducksalad | 60 | 95 | 95 | 85 | 95 | 95 | 95 | 90 | 85 | 90 | 70 | 90 | 90 | 85 |
| Rice | 10 | 15 | 30 | 55 | 30 | 25 | 40 | 20 | 10 | 0 | 25 | 0 | 15 | 0 |
| Sedge, Umbrella | 70 | 90 | 95 | 90 | 95 | 90 | 90 | 95 | 90 | 80 | 98 | 98 | 95 | 95 |

| | 250 g ai/ha Flood | | | | | | |
|---|---|---|---|---|---|---|---|
| | Compounds | | | | | | |
| | 57 | 58 | 59 | 60 | 61 | 63 | 64 |
| Barnyardgrass | 15 | 90 | 60 | 90 | 95 | 45 | 25 |
| Ducksalad | 90 | 80 | 80 | 90 | 75 | 95 | 80 |
| Rice | 10 | 60 | 40 | 35 | 20 | 45 | 15 |
| Sedge, Umbrella | 95 | 95 | 95 | 95 | 90 | 95 | 95 |

What is claimed is:

1. A compound selected from Formula 1, all stereoisomers, N-oxides, and salts thereof, 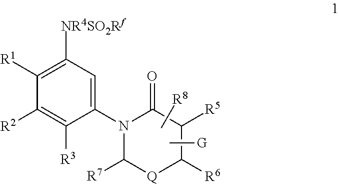
wherein
R¹ is H, C₁-C₇ alkyl, halogen, CN, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₃-C₇ haloalkynyl, C₂-C₇ alkoxyalkyl, C₁-C₇ alkoxy, C₁-C₅ alkylthio, C₂-C₃ alkoxycarbonyl or C₂-C₇ haloalkoxyalkyl;
R² is H, C₁-C₇ alkyl, halogen, CN, C₁-C₇ haloalkyl, C₁-C₇ alkoxy or C₁-C₅ alkylthio;
R³ is H, C₁-C₇ alkyl, halogen, CN, C₂-C₆ alkenyl, C₂-C₇ alkynyl, C₃-C₇ cycloalkyl, C₂-C₄ cyanoalkyl, C₁-C₇ haloalkyl, C₂-C₇ haloalkenyl, C₃-C₇ haloalkynyl, C₂-C₇ alkoxyalkyl, C₁-C₇ alkoxy, C₁-C₅ alkylthio, C₂-C₃ alkoxycarbonyl or C₂-C₇ haloalkoxyalkyl;
R⁴ is H, C(═O)R¹⁴, —C(═S)R¹⁴, —CO₂R¹⁴, —C(═O)SR¹⁴, —S(O)₂R¹⁴, C(═O)NR¹³R¹⁴, —S(O)₂NR¹³R¹⁴, CH₂OC(═O)OR¹⁴, CH₂OC(═O)NR¹³R¹⁴ or CH₂OC(═O)R¹⁴; or propargyl, allyl or benzyl;
R⁵ is H, C₂-C₆ alkenyl, C₂-C₇ haloalkenyl, C₂-C₆ alkynyl, C₃-C₇ cycloalkyl, C₄-C₇ cycloalkylalkyl, C₂-C₄ cyanoalkyl, C₁-C₇ haloalkyl, C₃-C₇ alkylthioalkyl, C₁-C₇ haloalkoxy, C₂-C₇ alkoxyalkyl or C₄-C₇ alkylcycloalkyl;
R⁶ is H, C₁-C₇ alkyl, halogen, CN, C₁-C₅ alkylthio, C₂-C₃ alkoxycarbonyl, C₂-C₆ alkenyl, C₂-C₆ alkynyl, C₃-C₇ cycloalkyl, C₄-C₇ cycloalkylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ haloalkoxyalkyl or C₄-C₇ alkylcycloalkyl;
R⁷ is H, C₁-C₇ alkyl, halogen, CN, C₁-C₅ alkylthio, C₂-C₃ alkoxycarbonyl, C₂-C₆ alkenyl, C₂-C₆ alkynyl, C₃-C₇ cycloalkyl, C₄-C₇ cycloalkylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ haloalkoxyalkyl or C₄-C₇ alkylcycloalkyl;
R⁸ is H, C₁-C₇ alkyl, C₂-C₆ alkenyl, C₂-C₆ alkynyl, C₃-C₇ cycloalkyl, C₄-C₇ cycloalkylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ alkoxyalkyl or C₄-C₇ alkylcycloalkyl;
Q is CHR⁹, O or a direct bond;
R⁹ is H, C₁-C₇ alkyl, halogen, CN, C₁-C₅ alkylthio, C₂-C₃ alkoxycarbonyl, C₂-C₆ alkenyl, C₂-C₆ alkynyl, C₃-C₇ cycloalkyl, C₄-C₇ cycloalkylalkyl, C₂-C₄ cyanoalkyl, C₁-C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ alkoxyalkyl, C₂-C₇ haloalkoxyalkyl or C₄-C₇ alkylcycloalkyl;
G is OR¹⁰, SR¹⁰, SOR¹⁰ or SO₂R¹⁰; or
G and R⁵ are taken together to form N—OR¹⁵;
R¹⁰ is H, C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₂-C₄ cyanoalkyl, C₄-C₇ alkylcycloalkyl, C₁-C₆ nitroalkyl, C₃-C₆ alkylcarboalkyl, C₃-C₆ alkoxycarboalkyl, C₂-C₇ haloalkoxyalkyl, benzyl or C₃-C₆ alkylcarboalkoxy; or
R¹⁰ is selected from the group consisting of 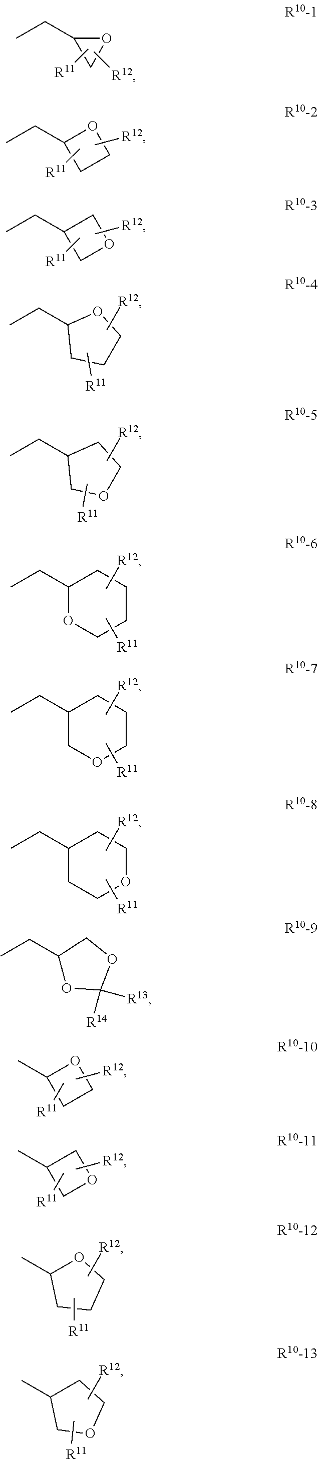 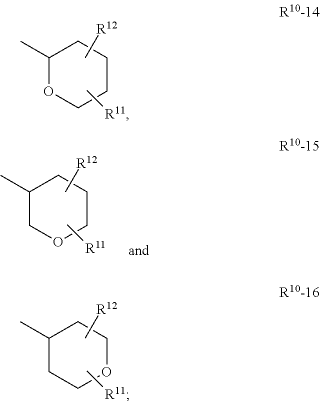
R¹¹ is H, C₁-C₇ alkyl, C₂-C₆ alkenyl, C₂-C₆ alkynyl, C₃-C₇ cycloalkyl, C₄-C₇ cycloalkylalkyl, C₇ haloalkyl, C₂-C₇ haloalkenyl, C₂-C₇ alkoxyalkyl, C₃-C₇ alkylthioalkyl, C₁-C₇ alkoxy, C₁-C₇ haloalkoxy, C₂-C₇ alkoxyalkyl or C₄-C₇ alkylcycloalkyl;
R¹² is H, C₁-C₇ alkyl, C₂-C₆ alkenyl, C₂-C₆ alkynyl, C₃-C₇ cycloalkyl, C₄-C₇ cycloalkylalkyl or C₇ haloalkyl;
each R¹³ and R¹⁴ is independently H, C₁-C₇ alkyl, C₂-C₆ alkenyl, C₂-C₆ alkynyl, C₃-C₇ cycloalkyl, C₄-C₇ cycloalkylalkyl, C₂-C₃ cyanoalkyl, C₁-C₇ haloalkyl, C₂-C₇ haloalkenyl, C₂-C₇ alkylalkoxyalkyl, C₃-C₇ alkylthioalkyl, C₁-C₇ alkoxy; C₂-C₇ alkoxyalkyl, C₄-C₇ alkylcycloalkyl, Ph or benzyl;
R^{f} is C₁-C₇ haloalkyl;
G and R⁸ can be attached to any ring carbon(s) with available valency, said ring is the cyclic amide ring shown in Formula 1;
each R¹¹ or R¹² can be attached to any ring carbon(s) with available valency, said ring is illustrated in R¹⁰-1 through R¹⁰-16 as above; and
R¹⁵ is H, C₁-C₆ alkyl, C₁-C₆ haloalkyl, C₂-C₆ alkenyl, C₂-C₆ alkynyl or C₄-C₇ cycloalkylalkyl.

2. The compound of claim 1 wherein
Q is direct bond;
R¹ is H, C₁-C₇ alkyl, halogen, C₂-C₆ alkenyl, C₂-C₆ alkynyl, C₃-C₇ cycloalkyl, C₁-C₇ haloalkyl;
R² is H, C₁-C₇ alkyl, halogen or CN;
R³ is H, C₁-C₇ alkyl, halogen, CN, C₁-C₇ alkoxy or C₁-C₇ haloalkyl;
R⁴ is H, C(═O)R¹⁴, —C(═S)R¹⁴, —CO₂R¹⁴, —C(═O)SR¹⁴, —S(O)₂R¹⁴, C(═O)NR¹³R¹⁴, —S(O)₂NR¹³R¹⁴, CH₂OC(═O)OR¹⁴, CH₂OC(═O)NR¹³R¹⁴ or CH₂OC(═O)R¹⁴;
R⁵ is H, C₂-C₆ alkenyl, C₂-C₇ haloalkenyl, C₂-C₆ alkynyl, C₃-C₇ cycloalkyl, C₄-C₇ cycloalkylalkyl, C₂-C₇ alkoxyalkyl or C₄-C₇ alkylcycloalkyl;
R⁶ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
R⁷ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
R⁸ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
G is OR¹⁰, SR¹⁰, SOR¹⁰ or SO₂R¹⁰;
R¹⁰ is H, C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₂-C₇ haloalkoxyalkyl, benzyl or C₄-C₇ alkylcycloalkyl;
R¹¹ is H or C₁-C₇ alkyl;
R¹² is H or C₁-C₇ alkyl;
each R¹³ and R¹⁴ is independently H, C₁-C₇ haloalkyl or C₁-C₇ alkyl; and
R^{f} is C₁-C₃ haloalkyl.

3. The compound of claim 2 wherein
R¹ is H, C₁-C₃ alkyl, halogen or C₃-C₄ cycloalkyl;
R² is H, Me, F, Cl or CN;
R³ is H, Me, F, Cl, —CN, OMe or CF₃;
R⁴ is H, SO₂CF₃, SO₂CH₃, CO₂Me, COMe, CH₂OCO-t-Bu, CH₂OCO-n-Bu, CH₂OCO-c-hexyl, CH₂OCO-c-pentyl, CH₂OCOCH₂CH₃, COMe, CH₂OCOPh, CH₂OCO-i-Bu, CH₂OCOMe, CH₂OCO-sec-Bu, CH₂OCO-n-Pr and CH₂OCO-i-Pr or (C═O)SMe;
R⁵ is H, C₄-C₇ cycloalkylalkyl or C₂-C₇ alkoxyalkyl;
R⁶ is H, C₁-C₇ alkyl or C₁-C₇ alkoxy;
R⁷ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
R⁸ is H, C₁-C₇ alkyl or C₁-C₇ alkoxy;
G is OR¹⁰ or SR¹⁰; and
R¹⁰ is C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₂-C₄ cyanoalkyl, C₃-C₇ alkylthioalkyl, benzyl or C₄-C₇ alkylcycloalkyl.

4. The compound of claim 3 wherein
R¹ is H, Me, halogen or cyclopropyl;
R² is H or F;
R³ is Me or F;
R⁴ is H, CH₂OCOR¹⁴ or —S(O)₂R¹⁴;
R⁵ is H;
R⁶ is H, Me or OMe;
R⁷ is H, Me or OMe;
R⁸ is H, Me or OMe;
G is OR¹⁰;
R¹⁰ is H, C₂-C₆ alkenyl, C₂-C₆ alkynyl, C₃-C₇ cycloalkyl, C₄-C₇ cycloalkylalkyl, C₄-C₇ halocycloalkylalkyl or C₄-C₇ alkylcycloalkyl.

5. (canceled)

6. (canceled)

7. The compound of claim 1 wherein
Q is CHR⁹;
R¹ is H, C₁-C₇ alkyl, halogen, C₂-C₆ alkenyl, C₂-C₆ alkynyl, C₃-C₇ cycloalkyl, C₁-C₇ haloalkyl;
R² is H, C₁-C₇ alkyl, halogen or CN;
R³ is H, C₁-C₇ alkyl, halogen, CN, C₁-C₇ alkoxy or C₁-C₇ haloalkyl;
R⁴ is H, C(═O)R¹⁴, —C(═S)R¹⁴, —CO₂R¹⁴, —C(═O)SR¹⁴, —S(O)₂R¹⁴, C(═O)NR¹³R¹⁴, —S(O)₂NR¹³R¹⁴, CH₂OC(═O)OR¹⁴, CH₂OC(═O)NR¹³R¹⁴ or CH₂OC(═O)R¹⁴;
R⁵ is H, C₂-C₆ alkenyl, C₂-C₇ haloalkenyl, C₂-C₆ alkynyl, C₃-C₇ cycloalkyl, C₄-C₇ cycloalkylalkyl, C₂-C₇ alkoxyalkyl or C₄-C₇ alkylcycloalkyl;
R⁶ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
R⁷ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
R⁸ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
G is OR¹⁰, SR¹⁰, SOR¹⁰ or SO₂R¹⁰;
R⁹ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
R¹⁰ is H, C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₂-C₇ haloalkoxyalkyl, benzyl or C₄-C₇ alkylcycloalkyl;
R¹¹ is H or C₁-C₇ alkyl;
R¹² is H or C₁-C₇ alkyl;
each R¹³ and R¹⁴ is independently H, C₁-C₇ haloalkyl or C₁-C₇ alkyl; and
R^{f} is C₁-C₃ haloalkyl.

8. The compound of claim 7 wherein
R¹ is H, C₁-C₃ alkyl, halogen or C₃-C₄ cycloalkyl;
R² is H, Me, F, Cl or CN;
R³ is H, Me, F, Cl, —CN, OMe or CF₃;
R⁴ is H, SO₂CF₃, SO₂CH₃, CO₂Me, COMe, CH₂OCO-t-Bu, CH₂OCO-n-Bu, CH₂OCO-c-hexyl, CH₂OCO-c-pentyl, CH₂OCOCH₂CH₃, COMe, CH₂OCOPh, CH₂OCO-i-Bu, CH₂OCOMe, CH₂OCO-sec-Bu, CH₂OCO-n-Pr and CH₂OCO-i-Pr or (C═O)SMe;
R⁵ is H, C₄-C₇ cycloalkylalkyl or C₂-C₇ alkoxyalkyl;
R⁶ is H, C₁-C₇ alkyl or C₁-C₇ alkoxy;
R⁷ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
R⁸ is H, C₁-C₇ alkyl or C₁-C₇ alkoxy;
G is OR¹⁰ or SR¹⁰;
R⁹ is H, C₁-C₇ alkyl or C₁-C₇ alkoxy; and
R¹⁰ is C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₂-C₄ cyanoalkyl, C₃-C₇ alkylthioalkyl or C₄-C₇ alkylcycloalkyl;

9. (canceled)

10. (canceled)

11. The compound of claim 1 wherein
Q is O;
R¹ is H, C₁-C₇ alkyl, halogen, C₂-C₆ alkenyl, C₂-C₆ alkynyl, C₃-C₇ cycloalkyl, C₁-C₇ haloalkyl;
R² is H, C₁-C₇ alkyl, halogen or CN;
R³ is H, C₁-C₇ alkyl, halogen, CN, C₁-C₇ alkoxy or C₁-C₇ haloalkyl;
R⁴ is H, C(═O)R¹⁴, —C(═S)R¹⁴, —CO₂R¹⁴, —C(═O)SR¹⁴, —S(O)₂R¹⁴, C(═O)NR¹³R¹⁴, —S(O)₂NR¹³R¹⁴, CH₂OC(═O)OR¹⁴, CH₂OC(═O)NR¹³R¹⁴ or CH₂OC(═O)R¹⁴;
R⁵ is H, C₂-C₆ alkenyl, C₂-C₇ haloalkenyl, C₂-C₆ alkynyl, C₃-C₇ cycloalkyl, C₄-C₇ cycloalkylalkyl, C₂-C₇ alkoxyalkyl or C₄-C₇ alkylcycloalkyl;
R⁶ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
R⁷ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
R⁸ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
G is OR¹⁰, SR¹⁰, SOR¹⁰ or SO₂R¹⁰;
R¹⁰ is H, C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₁-C₇ haloalkoxy, C₂-C₇ alkoxyalkyl, C₂-C₄ cyanoalkyl, C₁-C₇ haloalkyl, C₂-C₇ haloalkenyl, C₃-C₇ alkylthioalkyl, C₂-C₇ haloalkoxyalkyl, benzyl or C₄-C₇ alkylcycloalkyl;
R¹¹ is H or C₁-C₇ alkyl;
R¹² is H or C₁-C₇ alkyl;
each R¹³ and R¹⁴ is independently H, C₁-C₇ haloalkyl or C₁-C₇ alkyl; and
R^{f} is C₁-C₃ haloalkyl.

12. The compound of claim 11 wherein
R¹ is H, C₁-C₃ alkyl, halogen or C₃-C₄ cycloalkyl;
R² is H, Me, F, Cl or CN;
R³ is H, Me, F, Cl, —CN, OMe or CF₃;
R⁴ is H, SO₂CF₃, SO₂CH₃, CO₂Me, COMe, CH₂OCO-t-Bu, CH₂OCO-n-Bu, CH₂OCO-c-hexyl, CH₂OCO-c-pentyl, CH₂OCOCH₂CH₃, COMe, CH₂OCOPh, CH₂OCO-i-Bu, CH₂OCOMe, CH₂OCO-sec-Bu, CH₂OCO-n-Pr and CH₂OCO-i-Pr or (C═O)SMe;
R⁵ is H, C₄-C₇ cycloalkylalkyl or C₂-C₇ alkoxyalkyl;
R⁶ is H, C₁-C₇ alkyl or C₁-C₇ alkoxy;
R⁷ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
R⁸ is H, C₁-C₇ alkyl or C₁-C₇ alkoxy;
G is OR¹⁰ or SR¹⁰; and
R¹⁰ is C₂-C₆ alkenyl, C₂-C₆ alkynyl, C₃-C₇ cycloalkyl, C₃-C₇ halocycloalkyl, C₄-C₇ alkylcycloalkyl, C₄-C₇ cycloalkylalkyl, C₄-C₇ halocycloalkylalkyl, C₅-C₇ alkylcycloalkylalkyl, C₂-C₄ cyanoalkyl, C₃-C₇ alkylthioalkyl, benzyl or C₄-C₇ alkylcycloalkyl.

13. The compound of claim 12 wherein
R¹ is H, Me, halogen or cyclopropyl;
R² is H or F;
R³ is Me or F;
R⁴ is H, CH₂OCOR¹⁴ or —S(O)₂R¹⁴;
R⁵ is H;
R⁶ is H, Me or OMe;
R⁷ is H, Me or OMe;
R⁸ is H, Me or OMe;
G is OR¹⁰;
R¹⁰ is C₂-C₆ alkenyl, C₂-C₆ alkynyl, C₃-C₇ cycloalkyl, C₄-C₇ cycloalkylalkyl, C₄-C₇ halocycloalkylalkyl or C₄-C₇ alkylcycloalkyl.

14. (canceled)

15. (canceled)

16. The compound of claim 1 wherein
R¹ is H, C₁-C₇ alkyl, halogen, C₂-C₆ alkenyl, C₂-C₆ alkynyl, C₃-C₇ cycloalkyl, C₁-C₇ haloalkyl;
R² is H, C₁-C₇ alkyl, halogen or CN;
R³ is H, C₁-C₇ alkyl, halogen, CN, C₁-C₇ alkoxy or C₁-C₇ haloalkyl;
R⁴ is H, C(═O)R¹⁴, —C(═S)R¹⁴, —CO₂R¹⁴, —C(═O)SR¹⁴, —S(O)₂R¹⁴, C(═O)NR¹³R¹⁴, —S(O)₂NR¹³R¹⁴, CH₂OC(═O)OR¹⁴, CH₂OC(═O)NR¹³R¹⁴ or CH₂OC(═O)R¹⁴;
R⁶ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy;
R⁷ is H, C₁-C₇ alkyl, C₂-C₆ alkenyl, C₂-C₆ alkynyl, C₃-C₇ cycloalkyl, C₃-C₇ alkenylalkyl, C₃-C₇ alkynylalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
R⁸ is H, C₁-C₇ alkyl, C₂-C₆ alkenyl, C₂-C₆ alkynyl, C₃-C₇ cycloalkyl, C₂-C₄ cyanoalkyl, C₁-C₇ haloalkyl, C₂-C₇ haloalkenyl, C₂-C₇ alkoxyalkyl, C₁-C₇ alkoxy or C₁-C₇ haloalkoxy;
G and R⁵ are taken together to form N—OR¹⁵;
R¹¹ is H or C₁-C₇ alkyl;
R¹² is H or C₁-C₇ alkyl;
each R¹³ and R¹⁴ is independently H, C₁-C₇ haloalkyl or C₁-C₇ alkyl;
R^{f} is C₁-C₃ haloalkyl; and
R¹⁵ is H, C₁-C₆ alkyl, C₁-C₆ haloalkyl, C₂-C₆ alkenyl, C₂-C₆ alkynyl or C₄-C₇ cycloalkylalkyl.

17. The compound of claim 16 wherein
R¹ is H, C₁-C₃ alkyl, halogen or C₃-C₄ cycloalkyl;
R² is H, Me, F, Cl or CN;
R³ is H, Me, F, Cl, —CN, OMe or CF₃;
R⁴ is H, SO₂CF₃, SO₂CH₃, CO₂Me, COMe, CH₂OCO-t-Bu, CH₂OCO-n-Bu, CH₂OCO-c-hexyl, CH₂OCO-c-pentyl, CH₂OCOCH₂CH₃, COMe, CH₂OCOPh, CH₂OCO-i-Bu, CH₂OCOMe, CH₂OCO-sec-Bu, CH₂OCO-n-Pr and CH₂OCO-i-Pr or (C═O)SMe;
R⁶ is H, C₁-C₇ alkyl or C₁-C₇ alkoxy;
R⁷ is H, C₁-C₇ alkyl, C₃-C₇ cycloalkyl, C₁-C₇ haloalkyl, C₂-C₇ alkoxyalkyl, C₁-C₇ alkoxy or C₁-C₇ haloalkoxy; and
R⁸ is H, C₁-C₇ alkyl or C₁-C₇ alkoxy.

18. The compound of claim 17 wherein
R¹ is H, Me, halogen or cyclopropyl;
R² is H or F;
R³ is Me or F;
R⁴ is H, CH₂OCOR¹⁴ or —S(O)₂R¹⁴;
R⁶ is H, Me or OMe;
R⁷ is H, Me or OMe; and
R⁸ is H, Me or OMe.

19. The compound of claim 18 wherein
R¹ is H, Me, F, Cl, Br or cyclopropyl;
R⁴ is H, CH₂OCO-t-Bu or SO₂CF₃; and
R⁸ is H.

20. (canceled)

21. The compound of claim 16 wherein Q is direct bond.

22. The compound of claim 1 selected from the group consisting of
N-[5-[3-(cyclopentyloxy)-2-oxo-1-pyrrolidinyl]-2,4-dimethylphenyl]-1,1,1-
trifluoromethanesulfonamide (Compound 6);
[[5-[3-(cyclopentyloxy)-2-oxo-1-pyrrolidinyl]-2,4-
dimethylphenyl][(trifluoromethyl)sulfonyl]amino]methyl 2,2-dimethylpropanoate
(Compound 5)
N-[2,4-dimethyl-5-[2-oxo-3-(2-propyn-1-yloxy)-1-pyrrolidinyl]phenyl]-1,1,1-
trifluoromethanesulfonamide (Compound 1);
N-[5-[3-(cyclopropyloxy)-2-oxo-1-pyrrolidinyl]-2,4-dimethylphenyl]-1,1,1-
trifluoromethanesulfonamide (Compound 3);
[[5-[3-(cyclopropyloxy)-2-oxo-1-pyrrolidinyl]-2,4-
dimethylphenyl][(trifluoromethyl)sulfonyl]amino]methyl 2,2-dimethylpropanoate
(Compound 7);
[[5-[3-(cyclobutyloxy)-2-oxo-1-pyrrolidinyl]-2,4-
dimethylphenyl][(trifluoromethyl)sulfonyl]amino]methyl 2,2-dimethylpropanoate
(Compound 8);
N-[2,4-dimethyl-5-[2-oxo-3-(2-propen-1-yloxy)-1-pyrrolidinyl]phenyl]-1,1,1-
trifluoromethanesulfonamide (Compound 2); and
N-[5-[3-(cyclobutyloxy)-2-oxo-1-pyrrolidinyl]-2,4-dimethylphenyl]-1,1,1-
trifluoromethanesulfonamide (Compound 4).

23. The compound of claim 1 selected from the group consisting of
N-[5-[3-(cyclopentyloxy)-2-oxo-1-pyrrolidinyl]-2,4-dimethylphenyl]-1,1,1-
trifluoromethanesulfonamide (Compound 6);
[[5-[3-(cyclopentyloxy)-2-oxo-1-pyrrolidinyl]-2,4-
dimethylphenyl][(trifluoromethyl)sulfonyl]amino]methyl 2,2-dimethylpropanoate
(Compound 5)
N-[2,4-dimethyl-5-[2-oxo-3-(2-propyn-1-yloxy)-1-pyrrolidinyl]phenyl]-1,1,1-
trifluoromethanesulfonamide (Compound 1);
N-[5-[3-(cyclopropyloxy)-2-oxo-1-pyrrolidinyl]-2,4-dimethylphenyl]-1,1,1-
trifluoromethanesulfonamide (Compound 3);
[5-[3-(cyclopropyloxy)-2-oxo-1-pyrrolidinyl]-2,4-
dimethylphenyl][(trifluoromethyl)sulfonyl]amino]methyl 2,2-dimethylpropanoate
(Compound 7);
[[5-[3-(cyclobutyloxy)-2-oxo-1-pyrrolidinyl]-2,4-
dimethylphenyl][(trifluoromethyl)sulfonyl]amino]methyl 2,2-dimethylpropanoate
(Compound 8);
N-[2,4-dimethyl-5-[2-oxo-3-(2-propen-1-yloxy)-1-pyrrolidinyl]phenyl]-1,1,1-
trifluoromethanesulfonamide (Compound 2); and
N-[5-[3-(cyclobutyloxy)-2-oxo-1-pyrrolidinyl]-2,4-dimethylphenyl]-1,1,1-
trifluoromethanesulfonamide (Compound 4).
N-[5-[3-(Ethoxyimino)-2-oxo-1-pyrrolidinyl]-2,4-dimethylphenyl]-1,1,1-
trifluoromethanesulfonamide (Compound 12)
N-[2,4-Dimethyl-5-[2-oxo-3-[(2-propyn-1-yloxy)imino]-1-pyrrolidinyl]phenyl]-1,1,1-
trifluoromethanesulfonamide (Compound 13)
1,1,1-Trifluoro-N-[5-[3-(methoxyimino)-2-oxo-1-pyrrolidinyl]-2,4-
dimethylphenyl]methanesulfonamide (Compound 9)

24. A herbicidal composition comprising a compound of claim 1 and at least one component selected from the group consisting of surfactants, solid diluents and liquid diluents.

25. A herbicidal composition comprising a compound of claim 1, at least one additional active ingredient selected from the group consisting of other herbicides and herbicide safeners, and at least one component selected from the group consisting of surfactants, solid diluents and liquid diluents.

26. A herbicidal mixture comprising (a) a compound of claim 1, and (b) at least one additional active ingredient selected from (b1) photosystem II inhibitors, (b2) acetohydroxy acid synthase (AHAS) inhibitors, (b3) acetyl-CoA carboxylase (ACCase) inhibitors, (b4) auxin mimics, (b5) 5-enol-pyruvylshikimate-3-phosphate (EPSP) synthase inhibitors, (b6) photosystem I electron diverters, (b7) protoporphyrinogen oxidase (PPO) inhibitors, (b8) glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solanesyltransferase (HST) inhibitors, (b14) cellulose biosynthesis inhibitors, (b15) other herbicides including mitotic disruptors organic arsenicals, asulam, bromobutide, cinmethylin, cumyluron, dazomet, difenzoquat, dymron, etobenzanid, flurenol, fosamine, fosamine-ammonium, hydantocidin, metam, methyldymron, oleic acid, oxaziclomefone, pelargonic acid and pyributicarb, (b16) herbicide safeners, and salts of compounds of (b1) through (b16).

27. A method for controlling the growth of undesired vegetation comprising contacting the vegetation or its environment with a herbicidally effective amount of a compound of claim 1.

28. The method of claim 29 further comprising contacting the vegetation or its environment with a herbicidally effective amount of at least one additional active ingredient selected from (b1) through (b16) and salts of compounds of (b1) through (b16).

29. The compound of claim 17 wherein Q is direct bond.

30. The compound of claim 18 wherein Q is direct bond.

31. The compound of claim 19 wherein Q is direct bond.
